SEQUENCE LISTING (13 sequences)

SEQ ID NO: 1 - DNA, 20 bp, Artificial Sequence
  source: /note=“Description of Artificial Sequence Synthetic primer”
gaccccaaaatcagcgaaat

SEQ ID NO: 2 - DNA, 24 bp, Artificial Sequence
  source: /note=“Description of Artificial Sequence Synthetic primer”
tctggttactgccagttgaatctg

SEQ ID NO: 3 - DNA, 24 bp, Artificial Sequence
  source: /note=“Description of Artificial Sequence Synthetic probe”
accccgcattacgtttggtggacc

SEQ ID NO: 4 - DNA, 20 bp, Artificial Sequence
  source: /note=“Description of Artificial Sequence Synthetic primer”
ttacaaacattggccgcaaa

SEQ ID NO: 5 - DNA, 18 bp, Artificial Sequence
  source: /note=“Description of Artificial Sequence Synthetic primer”
gcgcgacattccgaagaa

SEQ ID NO: 6 - DNA, 23 bp, Artificial Sequence
  source: /note=“Description of Artificial Sequence Synthetic probe”
acaatttgcccccagcgcttcag

SEQ ID NO: 7 - protein, 9 aa, Artificial Sequence
  source: /note=“Description of Artificial Sequence Synthetic peptide”
VARLQSGFG

SEQ ID NO: 8 - protein, 14 aa, Artificial Sequence
  source: /note=“Description of Artificial Sequence Synthetic peptide”
KTSAVLQSGFRKME

SEQ ID NO: 9 - protein, 13 aa, Artificial Sequence
  source: /note=“Description of Artificial Sequence Synthetic peptide”
ESATLQSGLRKAK

SEQ ID NO: 10 - DNA, 1548 bp, Homo sapiens
atgcttttgagccagaatgccttcatcttcagatcacttaatttggttctcatggtgtatatcagcctcgtgtttggtatttcatatgattcgcctgattacacagatgaatcttgcactttcaagatatcattgcgaaatttccggtccatcttatcatgggaattaaaaaaccactccattgtaccaactcactatacattgctgtatacaatcatgagtaaaccagaagatttgaaggtggttaagaactgtgcaaataccacaagatcattttgtgacctcacagatgagtggagaagcacacacgaggcctatgtcaccgtcctagaaggattcagcgggaacacaacgttgttcagttgctcacacaatttctggctggccatagacatgtcttttgaaccaccagagtttgagattgttggttttaccaaccacattaatgtgatggtgaaatttccatctattgttgaggaagaattacagtttgatttatctctcgtcattgaagaacagtcagagggaattgttaagaagcataaacccgaaataaaaggaaacatgagtggaaatttcacctatatcattgacaagttaattccaaacacgaactactgtgtatctgtttatttagagcacagtgatgagcaagcagtaataaagtctcccttaaaatgcaccctccttccacctggccaggaatcagaatcagcagaatctgccaaaataggaggaataattactgtgtttttgatagcattggtcttgacaagcaccatagtgacactgaaatggattggttatatatgcttaagaaatagcctccccaaagtcttgaattttcataactttttagcctggccatttcctaacctgccaccgttggaagccatggatatggtggaggtcatttacatcaacagaaagaagaaagtgtgggattataattatgatgatgaaagtgatagcgatactgaggcagcgcccaggacaagtggcggtggctataccatgcatggactgactgtcaggcctctgggtcaggcctctgccacctctacagaatcccagttgatagacccggagtccgaggaggagcctgacctgcctgaggttgatgtggagctccccacgatgccaaaggacagccctcagcagttggaactcttgagtgggccctgtgagaggagaaagagtccactccaggacccttttcccgaagaggactacagctccacggaggggtctgggggcagaattaccttcaatgtggacttaaactctgtgtttttgagagttcttgatgacgaggacagtgacgacttagaagcccctctgatgctatcgtctcatctggaagagatggttgacccagaggatcctgataatgtgcaatcaaaccatttgctggccagcggggaagggacacagccaacctttcccagcccctcttcagagggcctgtggtccgaagatgctccatctgatcaaagtgacacttctgagtcagatgttgaccttggggatggttatataatgagatga

SEQ ID NO: 11 - protein, 515 aa, Homo sapiens [sequence omitted]

SEQ ID NO: 12 - DNA, 1548 bp, Artificial Sequence; identical to SEQ ID NO: 10
  source: /note=“Description of Artificial Sequence Synthetic polynucleotide”

SEQ ID NO: 13 - protein, 515 aa, Artificial Sequence [sequence omitted]

(19) United States
(12) Patent Application Publication
(10) Pub. No.: US 2023/0322779 A1
(43) Pub. Date: Oct. 12, 2023

(54) IMIDAZONAPHTHYRIDINES AND IMIDAZOPYRIDOPYRIMIDINES AS IFNAR2 AGONISTS FOR TREATING SARS-COV-2 INFECTIONS

(71) Applicant: GLAXOSMITHKLINE INTELLECTUAL PROPERTY DEVELOPMENT LIMITED, Middlesex (GB)

(72) Inventors: Jorge Esparza GORDILLO, Hertfordshire (GB); David John PAYNE, Collegeville, PA (US); Robert A. SCOTT, Hertfordshire (GB); Shihyun Kieffer YOU, Durham, NC (US)

(21) Appl. No.: 18/042,314

(22) PCT Filed: Sep. 2, 2021
(86) PCT No.: PCT/IB2021/058026

§ 371 Date: Feb. 21, 2023

Related U.S. Application Data
(60) Provisional application No. 63/074,425, filed on Sep. 3, 2020.

(51) Int. Cl. C07D 471/14; A61P 31/14

(52) U.S. Cl. CPC C07D 471/14; A61P 31/14

(57) ABSTRACT

The present disclosure relates to agonists of interferon alpha and beta receptor subunit 2 (IFNAR2) for use in the treatment or prevention of viral disease, particularly COVID-19. In particular embodiments, COVID-19 is associated with pneumonia or acute respiratory distress syndrome (ARDS). In other aspects, the subject being treated is undergoing extra-corporeal membrane oxygenation, mechanical ventilation, non-invasive ventilation, receiving oxygen therapy or receiving antiviral or steroid treatment.

### FIELD OF THE INVENTION

The present disclosure relates to agonists of interferon alpha and beta receptor subunit 2 (IFNAR2) for use in the treatment or prevention of viral disease, particularly COVID-19. In particular embodiments, COVID-19 is associated with pneumonia or acute respiratory distress syndrome (ARDS). In other aspects, the patient is undergoing extra-corporeal membrane oxygenation, mechanical ventilation, non-invasive ventilation, receiving oxygen therapy or receiving antiviral or steroid treatment.

### SEQUENCE LISTING

The instant application contains a Sequence Listing which has been submitted electronically in ASCII format and is hereby incorporated by reference in its entirety. Said ASCII copy, created on Aug. 16, 2021, is named PU66973_SL.txt and is 15,852 bytes in size.

### BACKGROUND TO THE INVENTION

COVID-19 was declared a Public Health Emergency of International Concern on 30 January 2020, following its emergence in China in November 2019. At the time of writing, over 216,664,634 cases and 4,505,400 deaths have been reported globally.

The infectious agent has been identified as a coronavirus (initially designated 2019-nCoV2 and more recently designated SARS-CoV-2, Severe Acute Respiratory Syndrome Coronavirus 2) capable of spreading by human to human transmission. Other coronaviruses that are pathogenic to humans are associated with mild clinical symptoms, with two notable exceptions: Severe Acute Respiratory Syndrome (SARS) Coronavirus (SARS-CoV) and Middle East Respiratory Syndrome (MERS) Coronavirus (MERS-CoV).

Coronaviruses consist of an enveloped single strand positive sense RNA genome of 26 to 32 kb in length. They are classified by phylogenetic similarity into four categories: α (e.g. 229E and NL-63), β (e.g. SARS-CoV-2, SARS-CoV, MERS-CoV and OC43), γ and δm. SARS-CoV-2 has also been reported to have 79% sequence identity to SARS-CoV, however certain regions of the SARS-CoV-2 genome exhibit greater or lesser degrees of conservation to SARS-CoV.

Structurally, SARS-CoV-2 has four main structural proteins including spike (S) glycoprotein, small envelope (E) glycoprotein, membrane (M) glycoprotein, and nucleocapsid (N) protein, and also several accessory proteins. The spike or S glycoprotein is a transmembrane protein with a molecular weight of about 150 kDa found in the outer portion of the virus. S protein forms homotrimers protruding in the viral surface and facilitates binding of envelope viruses to host cells by attraction with angiotensin-converting enzyme 2 (ACE2) expressed in lower respiratory tract cells. This glycoprotein is cleaved by the host cell furin-like protease into 2 subunits namely S1 and S2. Part S1 is responsible for the determination of the host virus range and cellular tropism with the receptor binding domain make-up while S2 functions to mediate virus fusion in transmitting host cells.

The nucleocapsid known as N protein is the structural component of SARS-CoV-2 localizing in the endoplasmic reticulum-Golgi region that structurally is bound to the nucleic acid material of the virus. Because the protein is bound to RNA, the protein is involved in processes related to the viral genome, the viral replication cycle, and the cellular response of host cells to viral infections. N protein is also heavily phosphorylated and suggested to lead to structural changes enhancing the affinity for viral RNA (see Structure of severe acute respiratory syndrome coronavirus 2 (Schoeman D. et al. Virol. J. 2019; 16:69 “Coronavirus envelope protein: current knowledge” and FIG. 1).

Another important part of the SARS-CoV-2 virus is the membrane or M protein, which is the most abundant structural protein and plays a role in determining the shape of the virus envelope. This protein can bind to all other structural proteins. Binding with M protein helps to stabilize nucleocapsids or N proteins and promotes completion of viral assembly by stabilizing N protein-RNA complex, inside the internal virion. The last component is the envelope or E protein which is the smallest protein in the SARS-CoV-2 structure that plays a role in the production and maturation of this virus.

Coronaviruses utilise membrane bound spike proteins (the S protein) to bind to a host cell surface receptor to gain cellular entry. Following entry into the host cell, the RNA genome is translated into two large polypeptides by the host ribosomal machinery. The polypeptides are processed by two proteases, the coronavirus main proteinase (3C-Like) and the papain-like proteinase to generate the proteins required for viral replication and packaging. To enter the host cell, SARS-CoV-2 binds to the angiotensin I converting enzyme 2 (ACE2) receptor that is highly expressed in the lower respiratory tract such as type II alveolar cells (AT2) of the lungs, upper esophagus and stratified epithelial cells, and other cells such as absorptive enterocytes from the ileum and colon, cholangiocytes, myocardial cells, kidney proximal tubule cells, and bladder urothelial cells. Therefore, patients who are infected with this virus not only experience respiratory problems such as pneumonia leading to Acute Respiratory Distress Syndrome (ARDS), but also experience disorders of heart, kidneys, and digestive tract.

At the time of writing, 48,635 coronavirus genomes of SARS-CoV-2 from around the world had been analysed (Daniele Mercatelli, Federico M. Giorgi. Geographic and Genomic Distribution of SARS-CoV-2 Mutations. Frontiers in Microbiology, 2020; 11 DOI: 10.3389/fmicb.2020.01800). All mutations were analzed and annotated with reference to the Wuhan genome (NC_045512.2) which has been designated the L strain or Glade.

Several clades (strains) have been identified which are designated L (original strain from Wuhan), S (named after the L to S amino acid change—the ORF8:L84S mutation), G (named after the D to G amino acid change in the Spike protein—the S:D614G mutation), V (named after the G to V amino acid change—the ORF3a:G251V mutation), and O (sequences not matching any of these criteria for the other clades). Clade G comprises two derivative clades, GH (characterized by the ORF3a:Q57H mutation) and GR (having a N:RG203KR mutation). Generally, clades G and GR are prevelant in Europe, and clades S and GH have been mostly observed in the Americas. The L Glade is mostly represented by sequences from Asia. At present, clades G and its derivatie offspring clades GH and GR are the most common among the sequences SAR-CoV-2 genomes, accounting for 74% of all world sequences, globally. The GR Glade, having both the Spike D614G and Nucleocapsid RG203KR mutations, is the most common representative of the SARS-CoV-2 genome population worldwide. The original viral strain, Glade L, continues to account for 7% of the sequenced genomes, and clades S and V have similar frenquencies in the global dataset of sequences.

Although several groups have confirmed the relatively low variability of SARS-CoV-2 genomes, it is not clear if the different fatalitiy rates or speed of transmission observed within different countries is related to differences in virulence between different clades.

### SUMMARY OF THE INVENTION

The present invention relates to compounds that act as enhancers of the host's immune response. The compounds are believed to up-regulate expression and/or activity of one or more of these proteins, thereby leading to better viral defense and/or treatment.

In a first aspect, the invention provides a compound of the invention as described herein, or a pharmaceutically acceptable salt thereof, for use in the treatment or prevention of a common cold in a subjected infected with human coronavirus or at risk of infection with human coronavirus.

In another aspect, the invention provides a compound of the invention as described herein, or a pharmaceutically acceptable salt thereof, for use in the treatment or prevention of SARS in a subjected infected with SARS-CoV or at risk of infection with SARS-CoV.

In another aspect, the invention provides a compound of the invention as described herein, or a pharmaceutically acceptable salt thereof, for use in the treatment or prevention of MERS in a subjected infected with MERS-CoV or at risk of infection with MERS-CoV.

In another aspect, the invention provides a compound of the invention as described herein, or a pharmaceutically acceptable salt thereof, for use in the treatment or prevention of COVID-19 in a subject infected with SARS-CoV-2 or at risk of infection with SARS-CoV-2.

In another aspect, the invention provides a compound of the invention as described herein, or a pharmaceutically acceptable salt thereof, for use in the treatment or prevention of human rhinovirus in a subjected infected with human rhinovirus or at risk of infection with human rhinovirus.

In another aspect, the invention provides a compound of the invention as described herein, or a pharmaceutically acceptable salt thereof, for use in the treatment or prevention of influenza virus in a subjected infected with influenza virus or at risk of infection with influenza virus.

In another aspect, the invention provides a compound of the invention as described herein, or a pharmaceutically acceptable salt thereof, for use in the treatment or prevention of human metapneumovirus in a subjected infected with human metapneumovirus or at risk of infection with human metapneumovirus.

In another aspect, the invention provides a compound of the invention as described herein, or a pharmaceutically acceptable salt thereof, for use in the treatment or prevention of parainfluenza virus in a subjected infected with parainfluenza virus or at risk of infection with parainfluenza virus.

In another aspect, the invention provides a compound of the invention as described herein, or a pharmaceutically acceptable salt thereof, for use in the treatment or prevention of human respiratory syncytial virus in a subjected infected with human respiratory syncytial virus or at risk of infection with human respiratory syncytial virus.

In another aspect, the invention provides a compound agonist of human interferon alpha and beta receptor subunit 2 (IFNAR2) (SEQ ID NO: 10, SEQ ID NO: 11) or IFNAR2 variant (e.g. SEQ ID NO: 12, SEQ ID NO: 13) for use in the treatment or prevention of COVID-19 in a subject infected with SARS-CoV-2 or at risk of infection with SARS-CoV-2 having the structure according to Formula (I):

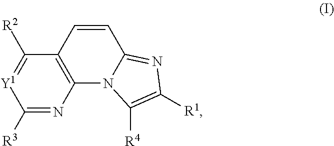

or a pharmaceutically acceptable salt thereof, wherein:
Y¹ is selected from the group consisting of CH and N;
R¹ is a 5-membered heteroaryl ring, wherein said 5-membered heteroaryl ring may have one to three heteroatoms selected from N, S, or O, and wherein said 5-membered heteroaryl ring may also be optionally substituted by one to three independent R⁵ groups;
R² is selected from (C₁-C₆)alkyl, (C₁-C₆)alkoxy, halo, and (C₄-C₆)aryl, wherein said R² group may be optionally substituted with one to three R⁵ groups;
R³ is selected from (C₁-C₆)alkyl, (C₁-C₆)alkoxy, halo, (C₄-C₆)aryl, and (C₃-C₆)cycloalkyl, wherein said R³ group may be optionally substituted with one to three R⁵ groups;
R⁴ is selected from hydrogen, (C₁-C₆)alkyl, and halo; and
R⁵ is halo or C₁-C₆ alkyl.

In another aspect, the invention provides a compound agonist of IFNAR2 (SEQ ID NO: 10, SEQ ID NO: 11) or IFNAR2 variant (e.g. SEQ ID NO: 12, SEQ ID NO: 13) or a pharmaceutically acceptable salt thereof, for use in the treatment or prevention of COVID-19 in a subject infected with SARS-CoV-2 or at risk of infection with SARS-CoV-2, selected from:
2-[2,4-bis(trifluoromethyl)imidazo[1,2-a]1,8-naphthyridin-8-yl]-1,3,4-oxadiazole;
2-[2-cyclopentyl-4-(trifluoromethyl)imidazo[1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[1,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo[1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo[1,2-a]1,8-naphthyridin-8-yl]-1,3,4-oxadiazole;
2-[2-phenyl-4-(trifluoromethyl)imidazo[1,2-a]1,8-naphthyridin-8-yl]-1,3,4-oxadiazole;
2-[9-chloro-2,4-bis(trifluoromethyl)imidazo[1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2,4-bis(trifluoromethyl)imidazo[1,2-a]1,8-naphthyridin-8-yl]-1,3-oxazole;
2-[2-chloro-4-(trifluoromethyl)imidazo[1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole.

In one embodiment, the invention provides a compound agonist of IFNAR2 (SEQ ID NO: 10, SEQ ID NO: 11) or IFNAR2 variant (e.g., SEQ ID NO: 12, SEQ ID NO: 13) or a pharmaceutically acceptable salt thereof, for use in the treatment or prevention of COVID-19, selected from:
2-[2,4-bis(trifluoromethyl)imidazo[1,2-a]1,8-naphthyridin-8-yl]-1,3,4-oxadiazole;
2-[2-cyclopentyl-4-(trifluoromethyl)imidazo[1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[1,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo[1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo[1,2-a]1,8-naphthyridin-8-yl]-1,3,4-oxadiazole;
2-[2-phenyl-4-(trifluoromethyl)imidazo[1,2-a]1,8-naphthyridin-8-yl]-1,3,4-oxadiazole;
2-[9-chloro-2,4-bis(trifluoromethyl)imidazo[1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2,4-bis(trifluoromethyl)imidazo[1,2-a]1,8-naphthyridin-8-yl]-1,3-oxazole;
2-[2-chloro-4-(trifluoromethyl)imidazo[1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole.

The invention also provides use of a compound agonist of IFNAR2 (SEQ ID NO: 10, SEQ ID NO: 11) or IFNAR2 variant (e.g., SEQ ID NO: 12, SEQ ID NO: 13) or pharmaceutically acceptable salt thereof, in the manufacture of a medicament for the treatment or prevention of COVID-19 selected from:
2-[2,4-bis(trifluoromethyl)imidazo[1,2-a]1,8-naphthyridin-8-yl]-1,3,4-oxadiazole;
2-[2-cyclopentyl-4-(trifluoromethyl)imidazo[1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[1 ,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo[1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo[1,2-a]1,8-naphthyridin-8-yl]-1,3,4-oxadiazole;
2-[2-phenyl-4-(trifluoromethyl)imidazo[1,2-a]1,8-naphthyridin-8-yl]-1,3,4-oxadiazole;
2-[9-chloro-2,4-bis(trifluoromethyl)imidazo[1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2 ,4-bis(trifluoromethyl)imidazo[1 ,2-a]1,8-naphthyridin-8-yl]-1,3-oxazole;
2-[2-chloro-4-(trifluoromethyl)imidazo[1,2-a]1,8-naphthyridin-8-yl]-1,3,4-oxadiazole;
2-[2 ,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole.

In one embodiment, the subject is infected with SARS-CoV-2. Methods for identifying subjects infected with SARS-CoV-2 are known in the art and are in current clinical use. In one embodiment, high-throughput sequencing or real-time reverse-transcriptase polymerase-chain-reaction (RT-PCR) assay of specimens, for example, nasal and pharyngeal swab specimens, may be used to identify subjects with active SARS-CoV-2 infection.

In one aspect, the invention provides a method for treating a viral infection in a subject comprising administering a therapeutically effective amount of a compound of the invention as described here, or a pharmaceutically acceptable salt thereof. In related embodiments, the viral infection is human coronavirus, SARS-CoV, MERS-CoV, or SARS-CoV-2.

In one aspect, the invention provides a method for treating a disease in a subject comprising administering an effective amount of a compound as described herein, or a pharmaceutically acceptable salt thereof. In related embodiments, the viral infection is human coronavirus, and the resulting disease is the common cold, the viral infection is SARS-CoV and the resulting disease is SARS, the viral infection is MERS-CoV, and the resulting disease is MERS, or the viral infection is SARS-CoV-2 and the disease is COVID-19.

In one aspect, the invention provides a method for treating or preventing COVID-19 in a subject infected with SARS-CoV-2 or at risk of infection with SARS-CoV-2, the method comprising:
administering a therapeutically effective amount of a compound agonist of IFNAR2 (SEQ ID NO: 10, SEQ ID NO: 11) or IFNAR2 variant thereof (e.g., SEQ ID NO: 12, SEQ ID NO: 13), or a pharmaceutically acceptable salt thereof having the structure according to Formula (I):

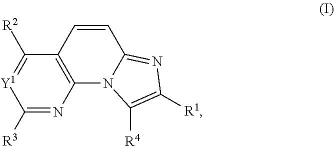

wherein:
Y¹ is selected from the group consisting of CH and N;
R¹ is a 5-membered heteroaryl ring, wherein said 5-membered heteroaryl ring may have one to three heteroatoms selected from N, S, or O, and wherein said 5-membered heteroaryl ring may also be optionally substituted by one to three independent R5 groups;
R² is selected from (C₁-C₆)alkyl, (C₁-C₆)alkoxy, halo, and (C₄-C₆)aryl, wherein said R² group may be optionally substituted with one to three R⁵ groups;
R³ is selected from (C₁-C₆)alkyl, (C₁-C₆)alkoxy, halo, (C₄-C₆)aryl, and (C₃-C₆)cycloalkyl, wherein said R3 group may be optionally substituted with one to three R5 groups;
R⁴ is selected from hydrogen, (C₁-C₆)alkyl, and halo; and
R⁵ is halo or C₁-C₆ alkyl.

In another aspect, the invention provides a method of treatment of a subject with COVID-19 with a therapeutically effective amount of a compound agonist of IFNAR2 (SEQ ID NO: 10, SEQ ID NO: 11) or IFNAR2 variant (e.g. SEQ ID NO: 12, SEQ ID NO: 13) or pharmaceutically acceptable salt thereof, selected from:
2-[2,4-bis(trifluoromethyl)imidazo[1,2-a]1,8-naphthyridin-8-yl]-1,3,4-oxadiazole;
2-[2-cyclopentyl-4-(trifluoromethyl)imidazo[1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[1,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo[1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo[1,2-a]1,8-naphthyridin-8-yl]-1,3,4-oxadiazole;
2-[2-phenyl-4-(trifluoromethyl)imidazo[1,2-a]1,8-naphthyridin-8-yl]-1,3,4-oxadiazole;
2-[9-chloro-2,4-bis(trifluoromethyl)imidazo[1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2,4-bis(trifluoromethyl)imidazo[1,2-a]1,8-naphthyridin-8-yl]-1,3-oxazole;
2-[2-chloro-4-(trifluoromethyl)imidazo[1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole.

In one embodiment, the subject is a human.

In addition, the invention provides a method for identifying subjects to be treated in accordance with the methods described herein, the method comprising a step of assaying a specimen from a subject for the presence of SARS-CoV-2 RNA. In some embodiments, the method is capable of identifying L strain SARS-CoV-2 RNA, S strain SARS-CoV-2 RNA, G strain SARS-CoV-2 RNA, GH strain SARS-CoV-2 RNA, GR strain SARS-CoV-2 RNA, V strain SARS-CoV-2 RNA, or O strain SARS-CoV-2 RNA. In some embodiments, where SARS-CoV-2 RNA is detected, the method may further comprises a treatment step as described herein.

In specific embodiments, the invention provides treating particular populations of patients with COVID-19, for example patients in high risk categories and patients with secondary conditions. In particular embodiments, the patient has pneumonia or acute respiratory distress syndrome. In additional embodiments, the patient is additionally undergoing extra-corporeal membrane oxygenation, mechanical ventilation, non-invasive ventilation, receiving oxygen therapy or receiving antiviral or steroid treatment.

In one embodiment, the uses and methods described herein are for treatment of a patient infected with a strain (clade) of SARS-CoV-2 selected from the L strain (clade), the S strain (clade), the G strain (clade), the GH strain (clade), the GR strain (clade), the V strain (clade), the O strain (clade), the GK strain (clade), GRY strain (clade) or the GV strain (clade) of SARS-CoV-2.

In one embodiment, the uses and methods described herein are for treatment of a patient infected with the L strain (clade) of SARS-CoV-2. In another embodiment, the uses and methods described herein are for treatment of a patient infected with the S strain (clade) of SARS-CoV-2. In another embodiment, the uses and methods described herein are for treatment of a patient infected with the G strain (clade) of SARS-CoV-2. In another embodiment, the uses and methods described herein are for treatment of a patient infected with the GH strain (clade) of SARS-CoV-2. In another embodiment, the uses and methods described herein are for treatment of a patient infected with the GR strain (clade) of SARS-CoV-2. In another embodiment, the uses and methods described herein are for treatment of a patient infected with the V strain (clade) of SARS-CoV-2. In another embodiment, the uses and methods described herein are for treatment of a patient infected with the O strain (clade) of SARS-CoV-2. In another embodiment, the uses and methods described herein are for treatment of a patient infected with the GK strain (clade) of SARS-CoV-2. In another embodiment, the uses and methods described herein are for treatment of a patient infected with the GRY strain (clade) of SARS-CoV-2. In another embodiment, the uses and methods described herein are for treatment of a patient infected with the GV strain (clade) of SARS-CoV-2.

In one embodiment, the uses and methods described herein are for treatment of a patient infected with any variant of SARS-CoV-2, e.g., the Alpha (B.1.1.7), Beta (B.1.351, B.1.351.2, B.1.351.3, B.1.427 and B.1.429), Delta (B.1.617.2, AY.1, AY.2, AY.3), Eta (B.1.525), Gamma (P.1, P.1.1, P.1.2) and Iota (B.1.526) variants. In one embodiment, the uses and methods described herein are for treatment of a patient infected with the Alpha (B.1.1.7) variant of SARS-CoV-2. In one embodiment, the uses and methods described herein are for treatment of a patient infected with the Beta (B.1.351, B.1.351.2, B.1.351.3, B.1.427 and B.1.429) variant of SARS-CoV-2. In one embodiment, the uses and methods described herein are for treatment of a patient infected with the Beta (B.1.351) variant of SARS-CoV-2. In one embodiment, the uses and methods described herein are for treatment of a patient infected with the Beta (B.1.351.2) variant of SARS-CoV-2. In one embodiment, the uses and methods described herein are for treatment of a patient infected with the Beta (B.1.351.3) variant of SARS-CoV-2. In one embodiment, the uses and methods described herein are for treatment of a patient infected with the Beta (B.1.427) variant of SARS-CoV-2. In one embodiment, the uses and methods described herein are for treatment of a patient infected with the Beta (B.1.429) variant of SARS-CoV-2. In one embodiment, the uses and methods described herein are for treatment of a patient infected with the Delta (B.1.617.2, AY.1, AY.2, AY.3) variant of SARS-CoV-2. In one embodiment, the uses and methods described herein are for treatment of a patient infected with the Delta (B.1.617.2) variant of SARS-CoV-2. In one embodiment, the uses and methods described herein are for treatment of a patient infected with the Delta (AY.1) variant of SARS-CoV-2. In one embodiment, the uses and methods described herein are for treatment of a patient infected with the Delta (AY.2) variant of SARS-CoV-2. In one embodiment, the uses and methods described herein are for treatment of a patient infected with the Delta (AY.3) variant of SARS-CoV-2. In one embodiment, the uses and methods described herein are for treatment of a patient infected with the Eta (B.1.525) variant of SARS-CoV-2. In one embodiment, the uses and methods described herein are for treatment of a patient infected with the Gamma (P.1, P.1.1, P.1.2) variant of SARS-CoV-2. In one embodiment, the uses and methods described herein are for treatment of a patient infected with the Gamma (P.1) variant of SARS-CoV-2. In one embodiment, the uses and methods described herein are for treatment of a patient infected with the Gamma (P.1.1) variant of SARS-CoV-2. In one embodiment, the uses and methods described herein are for treatment of a patient infected with the Gamma (P.1.2) variant of SARS-CoV-2. In one embodiment, the uses and methods described herein are for treatment of a patient infected with the Iota (B.1.526) variant of SARS-CoV-2.

In another embodiment, the subject is not infected with SARS-CoV-2 such that the use is for prevention of COVID-19. Subjects suitable for such prophylactic use include subjects in high risk categories, health care professionals and close contacts of subjects infected with SARS-CoV-2.

### BRIEF DESCRIPTION OF THE DRAWINGS

FIG. 1. Depicts the SARS-CoV-2 capsid.

FIG. 2. Depicts a regional association plot for COVID-19 hospitalization showing an association signal at the IFNAR2 locus on chromosome 21.

FIG. 3. Depicts a co-localization plot between COVID-19 hospitalization risk and IFNAR2 eQTL on regulatory T cells showing a negative correlation for these traits.

FIG. 4. Deicts a regional association plot for mumps risk showing an association signal at the IFNAR2 locus on chromosome 21.

FIG. 5. Depicts a regional association plot for shingles risk showing an association signal at the IFNAR2 locus on chromosome 21.

FIG. 6. Depicts a regional association plot for cold sores risk showing an association signal at the IFNAR2 locus on chromosome 21.

FIG. 7. Depicts a co-localization plot between mumps risk and IFNAR2 eQTL on CD4+ naïve T cells showing a negative correlation for these traits.

FIG. 8. Depicts a co-localization plot between shingles risk and IFNAR2 eQTL on CD4+ naïve T cells showing a negative correlation for these traits.

FIG. 9. Depicts a co-localization plot between cold sores risk and IFNAR2 eQTL on CD4+ naïve T cells showing a negative correlation for these traits. FIG. 10. Depicts the sequence of the IFNAR2 cDNA (SEQ ID NO: 12) with the encoded amino acid sequence (SEQ ID NO: 13) shown and the position of the missense variant rs1051393 indicated by the asterisk (*).

FIG. 11. Depicts the antiviral activity of the compounds of Example 1 and Example 9 against SARS-CoV-2 in Calu-3 cells. Each figure (FIG. 11(A) and FIG. 11(B)) contains side-by-side results from two independent experiments.

FIG. 12. Depicts the antiviral activity of the compound of Example 1 against SARS-CoV-2 in primary human bronchial cells cultured in air liquid interface at various time points, Day 2,

Day 4, Day 7, Day 9 and Day 11.

FIG. 13. Depicts the antiviral activity of the compound of Example 1 against a SARS-CoV-2 reporter virus in A549-hACE2 cells.

FIG. 14. Depicts the antiviral activity of the compound of Example 9 against a SARS-CoV-2 reporter virus in A549-hACE2 cells.

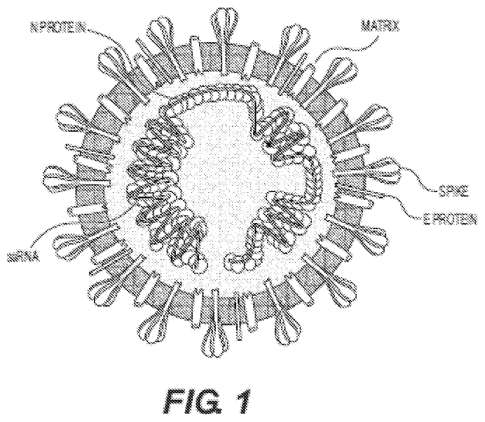

### DETAILED DESCRIPTION OF THE INVENTION

### Definitions

“Alkyl” refers to monovalent saturated aliphatic hydrocarbyl groups having from 1 to 14

carbon atoms and, in some embodiments, from 1 to 6 carbon atoms. “(C₁-C₆)alkyl” refers to alkyl groups having from x to y carbon atoms. This term includes, by way of example, linear and branched hydrocarbyl groups such as methyl (CH₃), ethyl (CH₃CH₂), n propyl (CH₃CH₂CH₂), isopropyl ((CH₃)₂CH), n butyl (CH₃CH₂CH₂CH₂), isobutyl ((CH₃)₂CHCH₂), sec butyl ((CH₃)(CH₃CH₂)CH), t butyl ((CH₃)₃C), n-pentyl (CH₃CH₂CH₂CH₂CH₂), and neopentyl ((CH₃)₃CCH₂).

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Alkoxy” refers to the group O alkyl wherein alkyl is defined herein. Alkoxy includes, by way of example, methoxy, ethoxy, n propoxy, isopropoxy, n butoxy, t butoxy, sec butoxy, and n pentoxy.

“Acyl” refers to the groups H C(O) , alkyl C(O) , alkenyl C(O) , alkynyl C(O), cycloalkyl C(O) , aryl C(O) , heteroaryl C(o) , and heterocyclic C(O) . Acyl includes the “acetyl” group CH₃C(O).

“Acyloxy” refers to the groups alkyl-C(O)O—, alkenyl-C(O)O—, alkynyl-C(O)O—, aryl-C(O)O—, cycloalkyl-C(O)O, heteroaryl C(O)O , and heterocyclic C(O)O .

“Amino” refers to the group —NR²¹R²² where R²¹ and R²² are independently selected from hydrogen, alkyl, alkenyl, alkynyl, aryl, cycloalkyl, heteroaryl, heterocyclic, —SO₂-alkyl, —SO₂-alkenyl, —SO₂-cycloalkyl, —SO₂-aryl, —SO₂-heteroaryl, and —SO₂-heterocyclic, and wherein R²¹ and R²² are optionally joined together with the nitrogen bound thereto to form a heterocyclic group. When R²¹ is hydrogen and R²² is alkyl, the amino group is sometimes referred to herein as alkylamino. When R²¹ and R²² are alkyl, the amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Aryl” or “aryl ring” refers to a 6- to 14-membered aromatic ring containing no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “Cycloalkyl” includes cycloalkenyl groups, such as cyclohexenyl. Examples of cycloalkyl groups include, for instance, adamantyl, cyclopropyl, cyclobutyl, cyclohexyl, cyclopentyl, cyclooctyl, cyclopentenyl, and cyclohexenyl. Examples of cycloalkyl groups that include multiple bicycloalkyl ring systems are bicyclohexyl, bicyclopentyl, bicyclooctyl, and the like. Two such bicycloalkyl multiple ring structures are exemplified and named below:

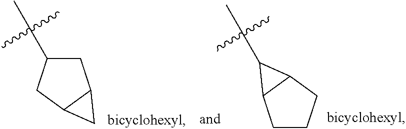 [SMILES: *C1CC2CC2C1 ; *C1C2CCCC12]

“(C₃-C₈)cycloalkyl” refers to cycloalkyl groups having 3 to 8 carbon atoms.

“Spiro cycloalkyl” refers to a 3- to 10-membered cyclic substituent formed by replacement of two hydrogen atoms at a common carbon atom in a cyclic ring structure or in an alkylene group having 2 to 9 carbon atoms, as exemplified by the following structure wherein the group shown here attached to bonds marked with wavy lines is substituted with a spiro cycloalkyl group:

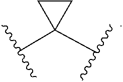 [SMILES: *C1(*)CC1]

“Fused cycloalkyl” refers to a 3- to 10-membered cyclic substituent formed by the replacement of two hydrogen atoms at different carbon atoms in a cycloalkyl ring structure, as exemplified by the following structure wherein the cycloalkyl group shown here contains bonds marked with wavy lines which are bonded to carbon atoms that are substituted with a fused cycloalkyl group:

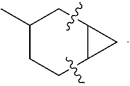

“Halo” or “halogen” refers to fluoro, chloro, bromo, and iodo.

“Haloalkoxy” refers to substitution of alkoxy groups with 1 to 5 (e.g., when the alkoxy group has at least 2 carbon atoms) or in some embodiments 1 to 3 halo groups (e.g., trifluoromethoxy).

“Hydroxy” or “hydroxyl” refers to the group —OH.

“Heteroaryl” and “heteroaryl ring” refers to 4- to 7-membered aromatic ring structure containing 1- to 6-heteroatoms in the ring, which heteroatoms are selected selected from oxygen, nitrogen, and sulfur. Heteroaryl and heteroaryl ring includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). More specifically the term heteroaryl includes, but is not limited to, pyridyl, furanyl, thienyl, thiazolyl, isothiazolyl, triazolyl, imidazolyl, imidazolinyl, isoxazolyl, pyrrolyl, pyrazolyl, pyridazinyl, pyrimidinyl, purinyl, phthalazyl, naphthylpryidyl, benzofuranyl, tetrahydrobenzofuranyl, isobenzofuranyl, benzothiazolyl, benzoisothiazolyl, benzotriazolyl, indolyl, isoindolyl, indolizinyl, dihydroindolyl, indazolyl, indolinyl, benzoxazolyl, quinolyl, isoquinolyl, quinolizyl, quianazolyl, quinoxalyl, tetrahydroquinolinyl, isoquinolyl, quinazolinonyl, benzimidazolyl, benzisoxazolyl, benzothienyl, benzopyridazinyl, pteridinyl, carbazolyl, carbolinyl, phenanthridinyl, acridinyl, phenanthrolinyl, phenazinyl, phenoxazinyl, phenothiazinyl, and phthalimidyl.

“Heterocyclic” or “heterocycle” or “heterocycloalkyl” or “heterocyclyl” refers to a saturated or partially saturated cyclic group having from 3 to 14 atoms and from 1 to 4 heteroatoms selected from nitrogen, sulfur, phosphorus or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In one embodiment, the nitrogen, phosphorus and/or sulfur atom(s) of the heterocyclic group are optionally oxidized to provide for the N oxide, phosphinane oxide, sulfinyl, sulfonyl moieties. More specifically the heterocyclyl includes, but is not limited to, tetrahydropyranyl, piperidinyl, piperazinyl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

Examples of heterocycle and heteroaryl groups include, but are not limited to, azetidine, pyrrole, imidazole, pyrazole, pyridine, pyrazine, pyrimidine, pyridazine, pyridone, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4 tetrahydroisoquinoline, 4,5,6,7 tetrahydrobenzo[b]thiophene, thiazole, thiazolidine, thiophene, benzo[b]thiophene, morpholine, thiomorpholine (also referred to as thiamorpholine), piperidine, pyrrolidine, and tetrahydrofuranyl. “Compound”, “compounds”, “chemical entity”, and “chemical entities” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Oxo” refers to a (═O) group.

“Oxazolidinone” refers to a 5-membered heterocyclic ring containing one nitrogen and one oxygen as heteroatoms and also contains two carbons and is substituted at one of the two carbons by a carbonyl group as exemplified by any of the following structures, wherein the oxazolidinone groups shown here are bonded to a parent molecule, which is indicated by a wavy line in the bond to the parent molecule:

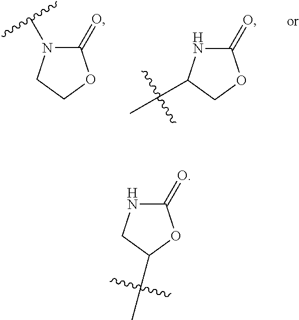 [SMILES: *N1CCOC1=O ; *C1COC(=O)N1 ; *C1CNC(=O)O1]

“Racemates” refers to a mixture of enantiomers. In an embodiment of the invention, the compounds described herein, or pharmaceutically acceptable salts thereof, are enantiomerically enriched with one enantiomer wherein all of the chiral carbons referred to are in one configuration. In general, reference to an enantiomerically enriched compound or salt, is meant to indicate that the specified enantiomer will comprise more than 50% by weight of the total weight of all enantiomers of the compound or salt.

“Solvate” or “solvates” of a compound refer to those compounds, as defined above, which are bound to a stoichiometric or non stoichiometric amount of a solvent. Solvates of a compound includes solvates of all forms of the compound. In certain embodiments, solvents are volatile, non toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol keto and imine enamine tautomers, or the tautomeric forms of heteroaryl groups containing a ring atom attached to both a ring NH moiety and a ring ═N moiety such as pyrazoles, imidazoles, benzimidazoles, triazoles, and tetrazoles.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

Wherever dashed lines occur adjacent to single bonds denoted by solid lines, then the dashed line represents an optional double bond at that position. Likewise, wherever dashed circles appear within ring structures denoted by solid lines or solid circles, then the dashed circles represent one to three optional double bonds arranged according to their proper valence taking into account whether the ring has any optional substitutions around the ring as will be known by one of skill in the art. For example, the dashed line in the structure below could either indicate a double bond at that position or a single bond at that position:

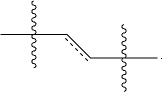

Similarly, ring A below could be a cyclohexyl ring without any double bonds or it could also be a phenyl ring having three double bonds arranged in any position that still depicts the proper valence for a phenyl ring. Likewise, in ring B below, any of X1-X5 could be selected from: C, CH, or CH₂, N, or NH, and the dashed circle means that ring B could be a cyclohexyl or phenyl ring or a N-containing heterocycle with no double bonds or a N-containing heteroaryl ring with one to three double bonds arranged in any position that still depicts the proper valence:

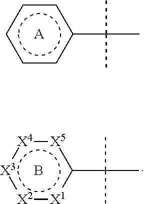

Where specific compounds or generic formulas are drawn that have aromatic rings, such as aryl or heteroaryl rings, then it will understood by one of skill in the art that the particular aromatic location of any double bonds are a blend of equivalent positions even if they are drawn in different locations from compound to compound or from formula to formula. For example, in the two pyridine rings (A and B) below, the double bonds are drawn in different locations, however, they are known to be the same structure and compound:

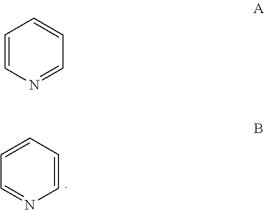 [SMILES: c1ccncc1 ; c1ccncc1]

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl) (alkyl) OC(O) . In a term such as “C(R^{x})₂”, it should be understood that the two R^{x} groups can be the same, or they can be different if Rx is defined as having more than one possible identity. In addition, certain substituents are drawn as —R^{x}R^{y}, where the “—” indicates a bond adjacent to the parent molecule and Ry being the terminal portion of the functionality. Similarly, it is understood that the above definitions are not intended to include impermissible substitution patterns (e.g., methyl substituted with 5 fluoro groups). Such impermissible substitution patterns are well known to the skilled artisan.

“SARS-CoV-2” is a beta coronavirus having greater than 90% sequence identity at the RNA level with any one of the sequences deposited in the China National Microbiological Data

Centre under accession number NMDC10013002, or greater than 90% sequence identity at the RNA level with any one of the sequences deposited in the Global Initiative on Sharing All Influenza Data (GISAID) under reference NC_045512.2 SARS-CoV-3 Wuhan genome (Coronaviridae Study Group of the International Committee on Taxonomy of Viruses, 2020). In another embodiment, the SARS-CoV-2 coronavirus has greater than 95% sequence identity at the RNA level with any one of the sequences deposited in the China National Microbiological Data Centre under accession number NMDC10013002 or with reference NC_045512.2 SARS-CoV-3 Wuhan genome (GISAID). In other embodiments, the SARS-CoV-2 coronavirus has greater than 96% sequence identity, greater than 97% sequence identity, greater than 98% sequence identity or greater than 99% sequence identity at the RNA level with any one of the sequences deposited in the China National Microbiological Data Centre under accession number NMDC10013002 or with reference NC_045512.2 SARS-CoV-3 Wuhan genome (GISAID). The definition of SARS-CoV-2 is intended to cover all strains of SARS-CoV-2 including the L, S, G, GH, GR, V and O clades (S Glade has a T at position 8782 and a C at position 28144; L Glade has a C at position 8782 and a T at position 28144; G Glade has a G at position 23403 (A23403G); GH Glade has a T at position 25563 (G25563T); GR Glade has a AAC for GGG starting at position 28881 (GGG28881AAC); Glade V has a Tat position 26144 (ORG2a:G251V); and O has sequence variations and mutations not defined by clades L, S, G, GH, GR or V), with numbering relating to the reference genome of 2019-nCoV-2 (NC_045512). Of note, the actual RNA base in the SARS-CoV-2 genome is U—uracil—but to be consistent with the original NCBI NC_045512.2 reference genomic notation, T is used here to characterize the genetic events.). The definition of SARS-CoV-2 also encompasses SARS-CoV-2 clades that have amino acid changes for Glade S (ORF8:L84S mutation), Glade G (S:D614G mutation), Glade GH (ORF3a:Q57H mutation), Glade GR (both S:D614G and N:RG203KR mutations), Glade V (ORF3a:G251V mutation), and Glade O (sequences and mutations not matching any of these criteria for the other clades).

“Percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query sequence may be described by a nucleic acid sequence identified herein.

“Percent identity” between a query amino acid sequence and a subject amino acid sequence is the “Identities” value, expressed as a percentage, that is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query sequence may be described by an amino acid sequence identified herein.

“COVID-19” refers to the collection of symptoms (e.g. https://www.cdc.gov/coronavirus/2019-ncov/symptoms-testing/symptoms.html) exhibited by patients infected with any strain or Glade of SARS-CoV-2. Symptoms typically include cough, fever and shortness of breath (dyspnoea).

“Subject” refers to mammals and includes humans and non-human mammals. In some embodiments, the subject is a human. In other embodiments, the subject is an animal such as dogs, cats, horses, cows, and livestock animals.

“Treating” or “treatment” of a disease in a patient refers to 1) preventing the disease from occurring in a patient that is predisposed or does not yet display symptoms of the disease;

2) inhibiting the disease or arresting its development; or 3) ameliorating or causing regression of the disease.

“Treatment of COVID-19” refers to a reduction in the viral load of SARS-CoV-2 and/or to a reduction in the viral titre of SARS-CoV-2, and/or to a reduction in the severity or duration of the symptoms of the disease. Viral load may be measured by a suitable quantitative RT-PCR assay from a specimen from the patient. In one embodiment, the specimen may be a specimen from the upper or lower respiratory tract (such as a nasopharyneal or oropharyngeal swab, sputum, lower respiratory tract aspirates, bronchoalveolar lavage, bronchial biopsy, transbronchial biopsy and nasopharyngeal wash/spirate or nasal aspirate) saliva or plasma. In a more particular embodiment, the specimen is saliva. The protocols of a number of quantitative RT-PCR assays are published on https://www.whoint/emergencies/diseases/novel-coronavirus-2019/technical-guidance/laboratory-guidance. In addition, Corman and colleagues have published primers and probes for use in such assays (Corman et al., European communicable disease bulletin, 2020, DOI: 10.2807/1560-7917). In one embodiment, the COVID-19 RdRp/He1 assay is used. This has been validated with clinical specimens and has a limit of detection of 1.8 TCID50/mL with genomic RNA and 11.2 RNA copies/reaction with in vitro RNA transcripts. (Chan et al., J Clin Microbiol., 2020, doi:10.1128/JCM.00310-20). Viral titre may be measured by assays well known in the art.

In one embodiment, treatment of COVID-19 refers to at least a 5 fold, 10 fold, 50 fold, 100 fold, 500 fold or 1000 fold reduction in the viral load (RNA copies/m1) measured by the same assay from a specimen from the same origin taken prior to treatment (baseline) and the end of the treatment period in a single patient. In another embodiment, treatment of COVID-19 refers to the situation where the mean viral load (RNA copies/ml) from specimens of the same origin from 30 patients measured in the same assay being reduced by at least 5 fold, 10 fold, 50 fold, 100 fold, 500 fold or 1000 fold at the end of the treatment period compared to baseline.

In one embodiment, treatment of COVID-19 refers to the viral load being decreased to below the limit of detection of the 19 RdRp/He1 assay at the end of the treatment period.

“Prevention of COVID-19” is interpreted in accordance with the usual meaning of the word “prevent”.

“High risk” subjects and “high risk” categories include the following: subjects of 60 years of age and over; smokers, subjects having a chronic medical condition including heart disease, lung disease, diabetes, cancer, obesity or high blood pressure; immunocompromised subjects such as subjects undergoing treatment for cancer or autoimmune diseases such as rheumatoid arthritis, systemic lupus erythematosus, multiple sclerosis and inflammatory bowel disease, subjects having a transplant and HIV positive individuals, including persons living in a community residence such as a dormatory, assisted living facility, nursing home, rehabilitation center and the like, persons having attended a gathering of 10, 25, 50, 100, 500, 1000 or more people not separated by —6-feet (2 meters) and/or not protected by a face mask or shield, and persons having travelled to, from or through a region with high levels of SARS-CoV-2 and COVID-19 cases.
“Close contacts” and “Close contacts of a subject infected with SARS-CoV-2” are defined as (i) persons living in the same household as the infected subject; (ii) persons having had direct or physical contact with the infected subject; (iii) persons having remained within two metres of an infected subject for longer than 15 minutes on or after the date on which symptoms were first reported by the subject.

### Identification of Subjects Infected with SARS-CoV-2

Subjects infected with SARS-CoV-2 may be identified by detection of viral RNA from SARS-CoV-2 from a specimen obtained from the subject. Without intending to be limiting, the specimen may be a specimen from the upper or lower respiratory tract (such as a nasopharyneal or oropharyngeal swab, sputum, lower respiratory tract aspirates, bronchoalveolar lavage and nasopharyngeal wash/spirate or nasal aspirate). Any known methods of RNA detection may be used, such as high-throughput sequencing or real-time reverse-transcriptase polymerase-chain-reaction (RT-PCR) assay. In one embodiment, the method comprises the following steps:
a) Isolating RNA from a specimen;
b) Reverse transcription of the RNA;
c) Amplification with forward and reverse primers in the presence of a probe; and
d) Detection of the probe;
   wherein the presence of SARS-CoV-2 is confirmed if the cycle threshold growth curves cross the threshold within 40 cycles.

In a more particular embodiment, step c) utilises the following:

Fwd Primer
(SEQ ID NO: 1)
5′ GACCCCAAAATCAGCGAAAT 3′
Rev Primer
(SEQ ID NO: 2)
5′ TCTGGTTACTGCCAGTTGAATCTG 3′
Probe
(SEQ ID NO: 3)
5′ FAM-ACCCCGCATTACGTTTGGTGGACC-BHQ-1 3′

In an alternative embodiment, step c) utilises the following:

Fwd Primer
(SEQ ID NO: 4)
5′ TTACAAACATTGGCCGCAAA 3′
Rev Primer
(SEQ ID NO: 5)
5′ GCGCGACATTCCGAAGAA 3′
Probe
(SEQ ID NO: 6)
5′ FAM-ACAATTTGCCCCCAGCGCTTCAG-BHQ-1 3′

These primers and probes are commercially available from Integrated DNA Technologies (Catalogue No. 10006606) and BioSearch Technologies (Catalogue No. KIT-nCoV-PP1-1000). Detailed instructions for performing real-time reverse-transcriptase polymerase-chain-reaction (RT-PCR) assay using these primers has been published by the CDC (https://www.cdc.gov/coronavirus/2019-nCoV/lab/index.html).

Accordingly, in one embodiment, the invention comprises a method for treating COVID-19 in a subject comprising a method of detecting viral RNA from SARS-CoV-2 from a specimen obtained from the subject and, where viral RNA is detected, a step of treating COVID-19 as described herein.

In one aspect, the invention provides a method for testing for SARS-CoV-2 in a subject and treating SARS-CoV-2 infection in the subject, which method comprises the following steps:
a) Isolating RNA from a specimen derived from a subject;
b) Reverse transcription of the RNA;
c) Amplification with forward and reverse primers in the presence of a probe; and
d) Detection of the probe;
   wherein the subject is defined as having SARS-CoV-2 infection if the cycle threshold growth curves cross the threshold within 40 cycles; and
e) the subject having SARS-CoV-2 infection with a therapeutically effective amount of a compound, or a pharmaceutically aceptable salt thereof, selected from:
2-[2,4-bis(trifluoromethyl)imidazo[1,2-a]1,8-naphthyridin-8-yl]-1,3,4-oxadiazole;
2-[2-cyclopentyl-4-(trifluoromethyl)imidazo[1,2-a]1,8-naphthyridin-8-yl]-1,3,4-oxadiazole;
2-[2-(propan-2-yl)-4-(trifluoromethyl)imidazo[1,2-a]1,8-naphthyrid in-8-yl]-1,3,4-oxadiazole;
5-[2,4-bis(trifluoromethyl)imidazo[1,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo[1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo[1,2-a]1,8-naphthyridin-8-yl]-1,3,4-oxadiazole;
2-[2-phenyl-4-(trifluoromethyl)imidazo[1,2-a]1,8-naphthyridin-8-yl]-1,3,4-oxadiazole;
2-[9-chloro-2,4-bis(trifluoromethyl)imidazo[1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2 4-bis(trifluoromethyl)imidazo[1,2-a]1,8-naphthyridin-8-yl]-1,3-oxazole;
2-[2-chloro-4-(trifluoromethyl)imidazo[1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole. In specific embodiments of this method, the subject is human, and the specimen and/or the primers and probe are as described above. The treatment may also be conducted as described herein.

In some embodiments, the method of identification of subjects infected with SARS-CoV-2 is capable of identifying whether the subject is infected with L strain SARS-CoV-2 RNA, S strain SARS-CoV-2 RNA, G strain SARS-CoV-2 RNA, GH strain SARS-CoV-2 RNA, GR strain SARS-CoV-2 RNA, V strain SARS-CoV-2 RNA, or O strain SARS-CoV-2 RNA. The method described herein could include a further step of sequencing amplified cDNA to identify whether the subject is infected with S strain, L strain, G strain, GH strain, GR strain, V strain or O strain.

In an alternative embodiment, subjects infected with SARS-CoV-2 may be identified by detection of a SARS-CoV-2 antigen or subject antibodies directed to SARS-CoV-2 in a sample of blood taken from the subject. In one embodiment, subjects infected with SARS-CoV-2 may be identified by detection of SARS-CoV-2 antigens in a sample of blood taken from the subject. Any suitable assay may be used. Kits for conducting such serological assays are already commercially available, e.g. from Biomerica and Pharmact. Details of performance of authorised serology tests is available on https://www.fda.gov/medical-devices/coronavirus-disease-2019-covid-19-emergency-use-authorizations-medical-devices/eua-authorized-serology-test-performance.

In one embodiment, the assay to identify subjects infected with SARS-CoV-2 comprises:
a) contacting at least one immobilised antigen from SARS-CoV-2 with blood from the subject; and
b) detection of a complex formed between subject antibodies directed to the immobilised antigen and the immobilised antigen;

where the the subject is identified to be infected with SARS-CoV-2 if a complex is detected in step b).

In a particular embodiment of this assay, the antigen from SARS-CoV-2 is selected from the N-protein and the S protein or fragments thereof. In a more particular embodiment, the antigen from SARS-CoV-2 is selected from the N-protein, the S1 domain of the S protein and the S2 domain of the S protein. In one embodiment, the assay comprises more than one immobilised antigen.

In one embodiment, there is a step of washing the immobilised antigen after step a) and before step b).

In one embodiment, the detection step b) comprises contacting the complex formed with a labelled antibody or antibodies recognising the same antigen or antigens followed by detection of the label. In a more particular embodiment, the complex is washed after addition of labelled antibody(ies) prior to detection of the label.

In one embodiment, the label is capable of producing a coloured product, enabling visual detection of the label.

In one embodiment, the assay is a lateral flow assay. In a more particular embodiment, the lateral flow assay has the immobilised antigen(s) on a dipstick.

In one embodiment, the invention provides a method for testing for SARS-CoV-2 in a subject and treating SARS-CoV-2 infection in the subject, which method comprises the following steps:
a) contacting at least one immobilised antigen from SARS-CoV-2 with blood from the subject; and
b) detecting a complex formed between subject antibodies directed to the immobilised antigen and the immobilised antigen;
   where the the subject is identified to be infected with SARS-CoV-2 if a complex is detected in step b); and
c) treating the subject having SARS-CoV-2 infection with a therapeutically effective amount of a compound, or a pharmaceutically aceptable salt thereof, selected from:
2-[2,4-bis(trifluoromethyl)imidazo[1,2-a]1,8-naphthyridin-8-yl]-1,3,4-oxadiazole;
2-[2-cyclopentyl-4-(trifluoromethyl)imidazo[1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[l ,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo[1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo[1,2-a]1,8-naphthyridin-8-yl]-1,3,4-oxadiazole;
2-[2-phenyl-4-(trifluoromethyl)imidazo[1,2-a]1,8-naphthyridin-8-yl]-1,3,4-oxadiazole;
2-[9-chloro-2,4-bis(trifluoromethyl)imidazo[1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2,4-bis(trifluoromethyl)imidazo[l ,2-a]1,8-naphthyridin-8-yl]-1,3-oxazole;
2-[2-chloro-4-(trifluoromethyl)imidazo[1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole.

In one embodiment, the invention provides a method for testing for SARS-CoV-2 in a subject and treating SARS-CoV-2 infection in the subject, which method comprises the following steps:
c) contacting at least one immobilised antigen from SARS-CoV-2 with blood from the subject; and
d) detecting a complex formed between subject antibodies directed to the immobilised antigen and the immobilised antigen;
   where the the subject is identified to be infected with SARS-CoV-2 if a complex is detected in step b); and
c) treating the subject having SARS-CoV-2 infection with a therapeutically effective amount of a compound, or a pharmaceutically acceptable salt thereof, selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole.

In one embodiment, the assay to identify subjects infected with SARS-CoV-2 comprises:
a) contacting an immobilised antibody recognising an antigen from SARS-CoV-2 with blood from the subject; and
b) detection of a complex formed between an antigen from SARS-CoV-2 and the immobilised antibody recognising said antigen;
   where the subject is identified to be infected with SARS-CoV-2 if a complex is detected in step b).

In specific embodiments of this method, the subject is human, and the assay is conducted as as described above. The treatment may also be conducted as described herein.

In a particular embodiment of this assay, the antigen from SARS-CoV-2 is selected from the N-protein and the S protein or fragments thereof. In a more particular embodiment, the the antigen from SARS-CoV-2 is selected from the N-protein, the S1 domain of the S protein and the S2 domain of the S protein. In one embodiment, the assay comprises more than one immobilised antibody, each antibody recognising a different antigen.

In one embodiment, there is a step of washing the immobilised antibody after step a) and before step b).

In one embodiment, the detection step b) comprises contacting the complex formed in step a) with labelled antibodies recognising the same antigen or antigens followed by detection of the label. In a more particular embodiment, step b) comprises a step of washing prior to detection of the label.

In one embodiment, the label is capable of producing a coloured product, enabling visual detection of the label.

In one embodiment, the assay is a lateral flow assay. In more particular embodiment, the lateral flow assay has the immobilised antibod(ies) on a dipstick.

In one embodiment, the invention provides a method for testing for and treating SARS-CoV-2 infection, which method comprises the following steps:
a) contacting an immobilised antibody recognising an antigen from SARS-CoV-2 with blood from the subject; and
b) detecting of a complex formed between an antigen from SARS-CoV-2 and the immobilised antibody recognising said antigen;
   wherein the subject is identified to be infected with SARS-CoV-2 if a complex is detected in step b); and
c) treating the subject having SARS-CoV-2 infection with a therapeutically effective amount of a compound, or a pharmaceutically aceptable salt thereof, selected from:
2-[2,4-bis(trifluoromethyl)imidazo[1,2-a]1,8-naphthyridin-8-yl]-1 ,3,4-oxadiazole;
2-[2-cyclopentyl-4-(trifluoromethyl)imidazo[1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[1,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo[1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo[1,2-a]1,8-naphthyridin-8-yl]-1,3,4-oxadiazole;
2-[2-phenyl-4-(trifluoromethyl)imidazo[1,2-a]1,8-naphthyridin-8-yl]-1,3,4-oxadiazole;
2-[9-chloro-2,4-bis(trifluoromethyl)imidazo[1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2,4-bis(trifluoromethyl)imidazo[1,2-a]1,8-naphthyridin-8-yl]-1,3-oxazole;
2-[2-chloro-4-(trifluoromethyl)imidazo[1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole. In specific embodiments of this method, the subject is human, and the assay is conducted as described above. The treatment may also be conducted as described herein.

In one aspect of the invention, the invention provides a method for testing for and treating SARS-CoV-2 infection, which method comprises the following steps:
c) contacting an immobilised antibody recognising an antigen from SARS-CoV-2 with blood from the subject; and
d) detecting of a complex formed between an antigen from SARS-CoV-2 and the immobilised antibody recognising said antigen;
   wherein the subject is identified to be infected with SARS-CoV-2 if a complex is detected in step b), and treating the subject having SARS-CoV-2 infection with a therapeutically effective amount of a compound, or a pharmaceutically acceptable salt thereof, selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole, for use in the prevention of COVID-19.

In one embodiment the disclosure provides a method for testing for and treating a SARS-CoV-2 infected patient, comprising the steps of: a) testing to determine a SARS-CoV-2 infected patient carries the rs1051393 allele as shown in SEQ ID NO: 12; and b) administering a compound according to the invention; whereby the SARS-CoV-2 infected patient is treated. In another embodiment of this method the compound may be an agonist or other stimulator of IFNAR2 activity.

In one embodiment the disclosure provides a method for testing for and treating a SARS-CoV-2 infected patient, comprising the steps of: a) testing to determine if a SARS-CoV-2 infected patient has a lower level of an IFNAR2 gene transcript in CD4+ naïve T cells relative to the mean IFNAR2 gene transcript level in CD4+ naïve T cells from SARS-CoV-2 infected patients with mild symptoms or no symptoms; and b) administering a compound according to the invention; whereby the SARS-CoV-2 infected patient is treated. In the embodiments of the disclosure the entire gene transcript shown in SEQ ID NO: 10 or SEQ ID NO: 12, or a portion of these may be detected. Other non-cDNA transcripts, or portions thereof, corresponding to IFNAR2 may also be detected (e.g., hnRNAs, fragments, and portions corresponding to introns, exons, etc.). In another embodiment of this method an agonist of IFNAR2 may be administered or another molecule that stimulates IFNAR2 activity may be administered. In another embodiment of this method the compound may be an agonist or other stimulator of IFNAR2 activity.

In one embodiment the disclosure provides a method for testing for and treating a SARS-CoV-2 infected patient, comprising the steps of: a) testing to determine if a SARS-CoV-2 infected patient has a lower level of an IFNAR2 protein in CD4+ naïve T cells relative to the mean IFNAR2 protein level in CD4+ naïve T cells from SARS-CoV-2 infected patients a mild symptoms or no symptoms and b) administering a compound according to the invention; whereby the SARS-CoV-2 infected patient is treated. In the embodiments of the disclosure the protein shown in SEQ ID NO: 11 or SEQ ID NO: 13, or a portion of these may be detected. Other portions of IFNAR2 may also be detected (e.g., via trypsinization, antibody recognition, etc.). In another embodiment of this method, and those described herein, an agonist of IFNAR2 may be administered or another molecule that stimulates IFNAR2 activity may be administered.

In one embodiment the disclosure provides a method of treating a SARS-CoV-2 infected patient, comprising the steps of: a) administering an agonist of IFNAR2; whereby the SARS-CoV-2 infected patient is treated. In another embodiment of this method an agonist of IFNAR2 may be administered or another molecule that stimulates IFNAR2 activity may be administered. Such molecules may be small molecules, upstream or downstream activators of the IFNAR2 pathway, constitutively active proteins, or over expressed proteins are useful in the methods described herein.

### Prophylactic Use

In one aspect of the invention, the invention provides a compound, or a pharmaceutically aceptable salt thereof, selected from:
2-[2,4-bis(trifluoromethyl)imidazo[1 ,2-a]1,8-naphthyridin-8-yl]-1,3,4-oxadiazole;
2-[2-cyclopentyl-4-(trifluoromethyl)imidazo[1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[1,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo[1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo[1,2-a]1,8-naphthyridin-8-yl]-1,3,4-oxadiazole;
2-[2-phenyl-4-(trifluoromethyl)imidazo[1,2-a]1,8-naphthyridin-8-yl]-1,3,4-oxadiazole;
2-[9-chloro-2,4-bis(trifluoromethyl)imidazo[1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2,4-bis(trifluoromethyl)imidazo[1,2-a]1,8-naphthyridin-8-yl]-1,3-oxazole;
2-[2-chloro-4-(trifluoromethyl)imidazo[1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole, for use in the prevention of COVID-19.

In one aspect of the invention, the invention provides a compound, or a pharmaceutically acceptable salt thereof, selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole, for use in the prevention of COVID-19.

In one embodiment, a specimen from the subject has been tested for SARS-CoV-2 RNA and no SARS-CoV-2 RNA was detected. In another embodiment, a specimen from the subject has not been tested for SARS-CoV-2 RNA. In more particular embodiments, the subject is in a high risk category (as defined herein), a health care professional or is a close contact of a patient infected with SARS-CoV-2 (as defined herein).

In one aspect of the invention there is provided a method of preventing COVID-19 in a subject at risk of an infection from SARS-CoV-2, the method comprising:
administering to the subject at risk of infection from SARS-CoV-2 a therapeutically effective amount of a compound, or a pharmaceutically acceptable salt thereof, selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole.

In more particular embodiments, the subject is in a high risk category (as defined herein), a health care professional or is a close contact of a subject infected with SARS-CoV-2 (as defined herein).

### Therapeutic Use

In one aspect of the invention, there is provided a compound, or a pharmaceutically aceptable salt thereof, selected from:
2-[2,4-bis(trifluoromethyl)imidazo[1,2-a]1,8-naphthyridin-8-yl]-1,3,4-oxadiazole;
2-[2-cyclopentyl-4-(trifluoromethyl)imidazo[1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[1,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo[1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo[1,2-a]1,8-naphthyridin-8-yl]-1,3,4-oxadiazole;
2-[2-phenyl-4-(trifluoromethyl)imidazo[1,2-a]1,8-naphthyridin-8-yl]-1,3,4-oxadiazole;
2-[9-chloro-2,4-bis(trifluoromethyl)imidazo[1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2,4-bis(trifluoromethyl)imidazo[1,2-a]1,8-naphthyridin-8-yl]-1,3-oxazole;
2-[2-chloro-4-(trifluoromethyl)imidazo[1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole, for use in the treatment of COVID-19.

In one aspect of the invention, the invention provides a compound, or a pharmaceutically acceptable salt thereof selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole, for use in the treatment of COVID-19.

In one embodiment, treatment is initiated within 24 hours of the onset of symptoms, or within 24 hours of being tested positive for SARS-CoV-2 infection, using for example, the method defined herein.

In one embodiment, the subject infected with SARS-CoV-2 is in a high risk category, as defined above.

In one embodiment, the COVID-19 in the subject infected with SARS-CoV-2 is associated with pneumonia. In a more particular embodiment, the subject infected with SARS-CoV-2 has a MuLBSTA score of ≥12, or a CURB-65 score of ≥2 or a PSI score ≥70. In other embodiments, the subject infected with SARS-CoV-2 meets one or more of the following criteria: pulse ≥125/minute, respiratory rate >30/minute, blood oxygen saturation ≤93%, PaO₂/FiO₂ ratio <300 mmHg, peripheral blood lymphocyte count <0.8*10^{9/}L, systolic blood pressure <90 mmHg, temperature <35 or ≥40° C., arterial pH<7.35, blood urea nitrogen ≥30 mg/dl, partial pressure of arterial O₂<60 mmHg, pleural effusion, lung infiltrates >50% of the lung field within 24-48 hours.

In one embodiment, the COVID-19 in the subject infected with SARS-CoV-2 is associated with acute respiratory distress syndrome. In a more particular embodiment, the subject infected with SARS-CoV-2 has a Murray Score of ≥2. In another embodiment, the subject infected with SARS-CoV-2 has a PaO₂/FiO₂ ratio ≤200 mmHg. In a more particular embodiment, the subject infected with SARS-CoV-2 has a PaO₂/FiO₂ ratio ≤100 mmHg. In another embodiment, the subject has a corrected expired volume per minute ≥10 L/min. Ire another embodiment, the subject infected with SARS-CoV-2 has respiratory system compliance ≤40 mL/cm H₂O. In another embodiment, the subject infected with SARS-CoV-2 has positive end-expiratory pressure ≥10 cm H₂O.

In particular embodiments, the subject infected with SARS-CoV-2 is undergoing extra-corporeal membrane oxygenation or mechanical ventilation, or receiving oxygen supplementation via a nasal cannula or simple mask. Where mechanical ventilation is used, this includes use of low tidal volumes (<6 mL/kg ideal body weight) and airway pressures (plateau pressure <30 cmH₂O). Where oxygen supplementation is via a nasal cannula, this may be delivered at 2 to 6 L/minute. Where oxygen supplementation is by a simple mask, this may be delivered at 5 to 10 L/minute.

In particular embodiments, the subject infected with SARS-CoV-2 is receiving anti-viral and or steroid treatment. In a more particular embodiment, the subject infected with SARS-CoV-2 is receiving an anti-viral agent. In even more particular embodiments, the anti-viral agent is selected from oseltamivir, remdesivir, ganciclovir, lopinavir, ritonavir and zanamivir. In one embodiment, the patient is receiving oseltamivir (75 mg every 12 h orally). In another embodiment, the subject infected with SARS-CoV-2 is receiving ganciclovir (0.25 g every 12 h intravenously). In another embodiment, the subject infected with SARS-CoV-2 is receiving lopinavir/ritonavir (400/100 mg twice daily orally).

In a further embodiment, the subject infected with SARS-CoV-2 is receiving 100 mg remdesivir daily intravenousiy.

In particular embodiments, the subject infected with SARS-CoV-2 is receiving treatment with steroids. In a more particular embodiment, the steroid is selected from dexamethasone, prednisone, methylprednisone and hydrocortisone.

In one embodiment, the subject infected with SARS-CoV-2 is receiving dexamethasone (6 mg once daily, orally or intravenously).

In one embodiment, the subject infected with SARS-CoV-2 is receiving prednisone (40 mg daily, in two divided doses).

In one embodiment, the subject infected with SARS-CoV-2 is receiving methylprednisone (32 mg daily, in two divided doses).

In one embodiment, the subject infected with SARS-CoV-2 is receiving hydrocortisone (160 mg daily, in two to four divided doses).

In one embodiment, the subject receiving treatment with any of the above steroids is a subject receiving mechanical ventilation or supplemental oxygen. In particular embodiments, the subject infected with SARS-CoV-2 is receiving convalescent plasma therapy, neutralizing mAb and/or polyclonal Ab therapy. Blood is collected from an ABO compatible donor after at least 3 weeks post onset of illness and 4 days post discharge and plasma is prepared by apheresis.

In one embodiment, the plasma has a neutralizing antibody titer of 1:640 or above, as measured by the plaque reduction neutralization test using SARS-CoV-2 virus. In one embodiment, the dose of convalescent plasma is 200 mL.

Antiviral response through interferon-alpha (IFNα) pathway activation, including via activation of JAK1/STAT pathway, has been described to be inhibited by human papillomavirus proteins E6 and E7 (See Stanley, M., Clinical Microbiology Rev. 25:2 215-222 (2012)), suggesting that the restoration/upregulation of the JAK1/STAT pathway activation as potentially being an effective antiviral approach for treating certain virus infections and ameliorating the resultant symptoms disease which may be caused by the virus infection. Without intending to be bound by any particular theory, compounds which activate the JAK1/STAT pathway or are agonists of IFNAR2, are expected to lead to effective therapies for treating various pathogenic viruses. In particular embodiments, the pathogenic virus is a virus from a viral family selected from the group consisting of Coronaviruses, Picornaviruses, Togaviruses, Flaviruses, Filoviruses, Paramyxoviruses, Bunyaviruses, Polyomaviruses, Adenoviruses, Hepadnaviridae Herpesviruses, Orthomyxoviruses, Pneumoviruses and Poxviruses.

In accordance with another embodiment of the present invention, there are provided compounds and methods for treating viral infections.

In accordance with another embodiment of the present invention, there are provided compounds and methods for treating viral infection, wherein said viral infection comprises one or more viruses from the Coronaviridae family including human coronavirus, Severe Acute Respiratory Syndrome coronavirus (SARS-CoV), Middle East Respiratory Syndrome coronavirus (MERS-CoV) and Severe Acute Respiratory Syndrome coronavirus 2 (SARS-CoV-2). The disease caused by these viruses are SARS (SARS-CoV), MERS (MERS-CoV) and COVID-19 (SARS-CoV-2). In accordance with another embodiment of the present invention, there are provided compounds and methods for treating viral infections, and the disease caused by such viral infection, wherein said viral infection is SARS-CoV, and the resulting disease is SARS, MERS-CoV, and the resulting disease is MERS, or SARS-CoV-2, and the disease is COVID-19.

In accordance with another embodiment of the present invention, there are provided compounds and methods for treating viral infections, and the disease caused by such viral infection, wherein said viral infection comprises one or more viruses from the Togavirus family.

In accordance with another embodiment of the present invention, there are provided compounds and methods for treating viral infection, wherein said viral infection comprises one or more viruses from the Picornavirus family selected from the group consisting of rhinovirus, poliovirus, Coxsackie virus, enteroviruses, Foot and Mouth Disease virus, Hepatitis A virus, and Norovirus.

In accordance with another embodiment of the present invention, there are provided compounds and methods for treating viral infections, and the disease caused by such viral infection, wherein said viral infection comprises one or more viruses from the Hepadnaviridae family, including, but not limited to hepatitis B virus.

In accordance with another embodiment of the present invention, there are provided compounds and methods for treating a virus from the infection by viruses of the family Picornaviridae, particularly human rhinovirus (HRV) type A, type B and type C.

In accordance with another embodiment of the present invention, there are provided compounds and methods for treating viral infections, wherein said viral infection comprises one or more viruses from the Togavirus family selected from the group consisting of Eastern Equine Encephalitis virus, Western Equine Encephalitis virus, Venezuelan Equine Encephalitis virus, Chikungunya virus, Ross River virus, Semliki Forest virus, and Sindbis virus.

In accordance with another embodiment of the present invention, there are provided compounds and methods for treating viral infections, wherein said viral infection comprises one or more viruses from the Flavivirus family.

In accordance with another embodiment of the present invention, there are provided compounds and methods for treating viral infections, wherein said viral infection comprises one or more viruses from the Flavivirus family selected from the group consisting of Dengue virus, Yellow fever virus, Japanese Encephalitis virus, St. Louis Encephalitis virus, West Nile virus, Tickborne encephalitis virus, Zika virus and Hepatitis C virus.

In accordance with another embodiment of the present invention, there are provided compounds and methods for treating viral infections, wherein said viral infection comprises one or more viruses from the Filovirus family.

In accordance with another embodiment of the present invention, there are provided compounds and methods for treating viral infections, wherein said viral infection comprises one or more viruses from the Filovirus family selected from the group consisting of Marburg virus and Ebola virus.

In accordance with another embodiment of the present invention, there are provided compounds and methods for treating viral infections, wherein said viral infection comprises one or more viruses from the Paramyxovirus family.

In accordance with another embodiment of the present invention, there are provided compounds and methods for treating viral infections, wherein said viral infection comprises one or more viruses from the negative strand RNA viruses selected from the group consisting of mumps virus, parainfluenza virus, Newcastle Disease virus, Measles morbillivirus, Nipah virus, respiratory syncytial virus (RSV), metapneumovirus, and influenza virus. In certain embodiments, the viral infection is the mumps virus and the disease caused by the viral infection is Mumps. In a particular embodiment, the present invention provides compound agonists of IFNAR2 for use in treating infection by viruses of the Family Paramyxoviridae particularly human parainfluenza virus type 3 (PIV3).

In accordance with another embodiment of the present invention, there are provided compounds and methods for treating viral infections, wherein said viral infection comprises one or more viruses from the Bunyavirus family.

In accordance with another embodiment of the present invention, there are provided compounds and methods for treating viral infections, wherein said viral infection comprises one or more viruses from the Bunyavirus family selected from the group consisting of Orthobunya viruses, Phleboviruses, Hantavirus, and Nairovirus.

In accordance with another embodiment of the present invention, there are provided compounds and methods for treating viral infections, wherein said viral infection comprises one or more viruses from the Polyomavirus family.

In accordance with another embodiment of the present invention, there are provided compounds and methods for treating viral infections, wherein said viral infection comprises one or more viruses from the Polyomavirus family selected from the group consisting of JC virus and BK virus.

In accordance with another embodiment of the present invention, there are provided compounds and methods for treating viral infections, wherein said viral infection comprises one or more viruses from the Adenovirus family.

In accordance with another embodiment of the present invention, there are provided compounds and methods for treating viral infections, wherein said viral infection comprises one or more viruses from the Herpes virus family.

In accordance with another embodiment of the present invention, there are provided compounds and methods for treating disease caused by or associated with a viral infection, wherein said viral infection comprises one or more viruses from the Herpes virus family selected from the group consisting of HHV-1 (HSV-1), HHV-2 (HSV-2), HHV-3 (VZV), HHV-4 (EBV), HHV-5 (CMV), HHV-8 (KSV), and B virus. In certain embodiments, the disease is selected from oral herpes (cold sores) cause by herpes simplex 1 (HSV-1) virus infection, and genital herpes cause by HSV-1 and/or caused by herpes simplex 2 (HSV-2).

In accordance with another embodiment of the present invention, there are provided compounds and methods for treating viral infections, wherein said viral infection comprises one or more viruses from the Orthomyxovirus family.

In accordance with another embodiment of the present invention, there are provided compounds and methods for treating disease caused by or associated with a viral infection, wherein said viral infection comprises one or more viruses from the Orthomyxovirus family selected from the group consisting of Influenza virus type A and Influenza virus type B. In one embodiment, the disease is selected from Influenza virus type A. In one embodiment, the disease is selected from Influenza virus type B.

In accordance with another embodiment of the present invention, there are provided compounds and methods for treating viral infections, wherein said viral infection comprises one or more viruses from the Pneumovirus family.

In accordance with another embodiment of the present invention, there are provided compounds and methods for treating disease caused by or associated with a viral infection, wherein said viral infection comprises one or more viruses from the Pneumovirus family selected from the group consisting of human metapneumovirus (MPV) and human respiratory syncytial virus (RSV-A and RSV-B). In one embodiment, the disease is selected from human metapneumovirus (MPV). In one embodiment, the disease is selected from human respiratory syncytial virus (RSV-A and RSV-B). In one embodiment, the disease is RSV-A. In one embodiment, the disese is RSV-B.

In accordance with another embodiment of the present invention, there are provided compounds and methods for treating viral infections, wherein said viral infection comprises one or more viruses from the Poxvirus family.

In accordance with another embodiment of the present invention, there are provided compounds and methods for treating viral infections, wherein said viral infection comprises one or more viruses from the Poxvirus family selected from the group consisting of monkeypox and Variola virus (smallpox).

In accordance with another embodiment of the present invention, there are provided compounds and methods for treating and preventing viral infections, wherein said viral infection comprises one or more viruses from the Papillomavirus family. Human papillomavirus (HPV) is a virus from the papillomavirus family that is capable of infecting humans.

In another aspect, the invention provides a compound agonist of IFNAR2 (SEQ ID NO: 10, SEQ ID NO: 11) or IFNAR2 variant (e.g. SEQ ID NO: 12, SEQ ID NO: 13), or pharmaceutically acceptable salt thereof, for use in the treatment or prevention of a disease associated with a coronavirus infection in a subject infected with a coronavirus or at risk of infection with a coronavirus, wherein the compound, or a pharmaceutically acceptable salt thereof, agonist of IFNAR2 or IFNAR2 variant is selected from:
2-[2,4-bis(trifluoromethyl)imidazo[1,2-a]1,8-naphthyridin-8-yl]-1,3,4-oxadiazole;
2-[2-cyclopentyl-4-(trifluoromethyl)imidazo[1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[1,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo[1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo[1,2-a]1,8-naphthyridin-8-yl]-1,3,4-oxadiazole;
2-[2-phenyl-4-(trifluoromethyl)imidazo[1,2-a]1,8-naphthyridin-8-yl]-1,3,4-oxadiazole;
2-[9-chloro-2,4-bis(trifluoromethyl)imidazo[1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2,4-bis(trifluoromethyl)imidazo[1,2-a]1,8-naphthyridin-8-yl]-1,3-oxazole;
2-[2-chloro-4-(trifluoromethyl)imidazo[1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole, for use in the treatment or prevention of a disease associated with a coronavirus infection.

The invention also provides use of a compound agonist of IFNAR2 (SEQ ID NO: 10, SEQ ID NO: 11) or pharmaceutically acceptable salt thereof, selected from:
2-[2,4-bis(trifluoromethyl)imidazo[1,2-a]1,8-naphthyridin-8-yl]-1,3,4-oxadiazole;
2-[2-cyclopentyl-4-(trifluoromethyl)imidazo[1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[1,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo[1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo[1,2-a]1,8-naphthyridin-8-yl]-1,3,4-oxadiazole;
2-[2-phenyl-4-(trifluoromethyl)imidazo[1,2-a]1,8-naphthyridin-8-yl]-1,3,4-oxadiazole;
2-[9-chloro-2,4-bis(trifluoromethyl)imidazo[1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2,4-bis(trifluoromethyl)imidazo[1,2-a]1,8-naphthyridin-8-yl]-1,3-oxazole;
2-[2-chloro-4-(trifluoromethyl)imidazo[1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole, in the manufacture of a medicament for the treatment or prevention of COVID-19.

In an embodiment, the coronavirus is SARS, MERS or SARS-CoV-2.

In one aspect there is provided a method for treating or preventing COVID-19 in a subject infected with SARS-CoV-2 or at risk of infection with SARS-CoV-2, the method comprising administering a therapeutically effective amount of a compound, or a pharmaceutically acceptable salt thereof, which is selected from:
2-[2,4-bis(trifluoromethyl)imidazo[1,2-a]1,8-naphthyridin-8-yl]-1,3,4-oxadiazole;
2-[2-cyclopentyl-4-(trifluoromethyl)imidazo[1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[1,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo[1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo[1,2-a]1,8-naphthyridin-8-yl]-1,3,4-oxadiazole;
2-[2-phenyl-4-(trifluoromethyl)imidazo[1,2-a]1,8-naphthyridin-8-yl]-1,3,4-oxadiazole;
2-[9-chloro-2,4-bis(trifluoromethyl)imidazo[1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2,4-bis(trifluoromethyl)imidazo[1,2-a]1,8-naphthyridin-8-yl]-1,3-oxazole;
2-[2-chloro-4-(trifluoromethyl)imidazo[1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole.

In one aspect there is provided a method for treating or preventing COVID-19 in a subject infected with SARS-CoV-2 or at risk of infection with SARS-CoV-2, the method comprising administering a therapeutically effective amount of a compound, or a pharmaceutically acceptable salt thereof, which is selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole.

In one embodiment, the method comprises administering a therapeutically effective amount of a compound, or a pharmaceutically acceptable salt thereof, selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole,
   wherein the subject is at risk of infection with SARS-CoV-2 and the method comprises prevention of COVID-19 in the subject at risk of infection with SARS-CoV-2. In one particular embodiment, the subject is: a close contact of a patient infected with SARS-CoV-2, in a high risk category; or a healthcare professional.

In one embodiment, the method comprises administering a therapeutically effective amount of a compound, or pharmaceutically acceptable salt thereof, selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole,
   wherein the subject is infected with SARS-CoV-2 and the method comprises treating COVID-19 in the subject infected with SARS-CoV-2. In a particular embodiment, the subject was identified as being infected with SARS-CoV-2, by detection of viral RNA from SARS-CoV-2 from a specimen obtained from the subject.

In one embodiment, the subject infected with SARS-CoV-2 is infected with a strain (clade) of SAR-CoV-2 selected from the L strain, the S strain, the G strain, the GH strain, the GR strain, the V strain or the O strain of SARS-CoV-2. In a particular embodiment, the subject infected with SARS-CoV-2 is infected with the L strain of SARS-CoV-2. In a particular embodiment, the subject infected with SARS-CoV-2 is infected with the S strain of SARS-CoV-2. In a particular embodiment, the subject infected with SARS-CoV-2 is infected with the G strain of SARS-CoV-2. In a particular embodiment, the subject infected with SARS-CoV-2 is infected with the GH strain of SARS-CoV-2. In a particular embodiment, the subject infected with SARS-CoV-2 is infected with the GR strain of SARS-CoV-2. In a particular embodiment, the subject infected with SARS-CoV-2 is infected with the V strain of SARS-CoV-2. In a particular embodiment, the subject infected with SARS-CoV-2 is infected with the O strain of SARS-CoV-2.

In one embodiment, the method comprises administering a therapeutically effective amount of a compound, or a pharmaceutically acceptable salt thereof, selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole,
   wherein COVID-19 in the subject infected with SARS-CoV-2 is associated with pneumonia. In a particular embodiment, COVID-19 in the subject infected with SARS-CoV-2 is associated with acute respiratory distress syndrome.

In one embodiment, the subject infected with SARS-CoV-2 is undergoing extra-corporeal membrane oxygenation, mechanical ventilation, non-invasive ventilation, or receiving oxygen therapy.

In one embodiment, the subject infected with SARS-CoV-2 is receiving anti-viral and/or steroid treatment. In a particular embodiment, the subject infected with SARS-CoV-2 is receiving an anti-viral agent. In another particular embodiment, the anti-viral agent is selected from remdesivir, ganciclovir, lopinavir, oseltamivir ritonavir and zanamivir. In one embodiment, the subject infected with SARS-CoV-2 is receiving 100 mg remdesivir daily intravenously. In a particular embodiments, the subject infected with SARS-CoV-2 is receiving treatment with steroids. In another particular embodiment, the steroid is selected from dexamethasone, prednisone, methylprednisone and hydrocortisone. In one embodiment, the subject infected with SARS-CoV-2 is receiving dexamethasone (6 mg once daily, orally or intravenously), in one embodiment, the subject receiving treatment with steroids is a patient receiving mechanical ventilation or supplemental oxygen. In a particular embodiment, the subject infected with SARS-CoV-2 is receiving convalescent plasma therapy, neutralizing mAb and/or polyclonal Ab therapy.

In one embodiment, the method comprises administering a therapeutically effective amount of a compound, or a pharmaceutically acceptable salt thereof, selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole,
   wherein the compound, or pharmaceutically acceptable salt thereof, is administered via inhalation.

### Compounds

2-[2,4-bis(trifluoromethyl)imidazo[1,2-a]1,8-naphthyridin-8-yl]-1,3,4-oxadiazole free base has the following structure:

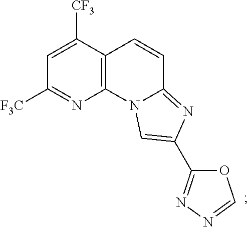 [SMILES: FC(F)(F)c1cc(C(F)(F)F)c2ccc3nc(-c4nnco4)cn3c2n1]

2-[2-cyclopentyl-4-(trifluoromethyl)imidazo[1,2-a]1,8-naphthyridin-8-yl]-1,3,4-oxadiazole free base has the following structure:

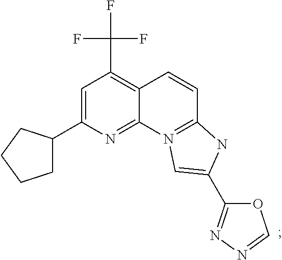

2-[2-(propan-2-yl)-4-(trifluoromethyl)imidazo[1,2-a]1,8-naphthyridin-8-yl]-1,3,4-oxadiazole free base has the following structure:

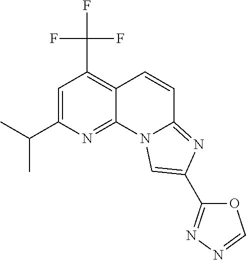 [SMILES: CC(C)c1cc(C(F)(F)F)c2ccc3nc(-c4nnco4)cn3c2n1]

5-[2,4-bis(trifluoromethyl)imidazo[1,2-a]1,8-naphthyridin-8-yl]-1,3-oxazole free base has the following structure:

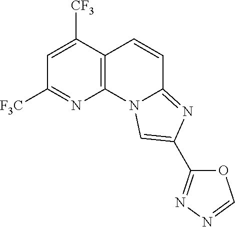 [SMILES: FC(F)(F)c1cc(C(F)(F)F)c2ccc3nc(-c4nnco4)cn3c2n1]

2-[2-cyclopropyl-4-(trifluoromethyl)imidazo[1,2-a]1,8-naphthyridin-8-yl]-1,3,4-oxadiazole free base has the following structure:

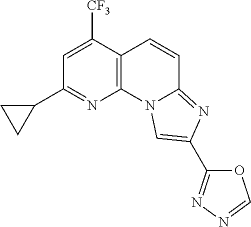 [SMILES: FC(F)(F)c1cc(C2CC2)nc2c1ccc1nc(-c3nnco3)cn12]

2-[2-methyl-4-(trifluoromethyl)imidazo[1,2-a]1,8-naphthyridin-8-yl]-1,3,4-oxadiazole free base has the following structure:

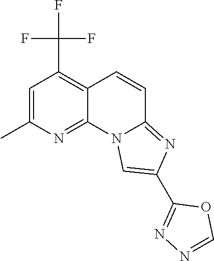 [SMILES: Cc1cc(C(F)(F)F)c2ccc3nc(-c4nnco4)cn3c2n1]

2-[9-methyl-2,4-bis(trifluoromethyl)imidazo[1,2-a]1,8-naphthyridin-8-yl]-1,3,4-oxadiazole free base has the following structure:

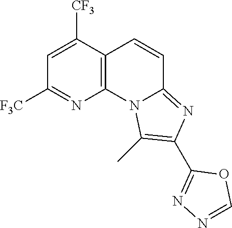 [SMILES: Cc1c(-c2nnco2)nc2ccc3c(C(F)(F)F)cc(C(F)(F)F)nc3n12]

2-[2-ethoxy-4-(trifluoromethyl)imidazo[1,2-a]1,8-naphthyridin-8-yl]-1,3,4-oxadiazole free base has the following structure:

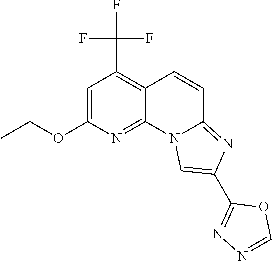 [SMILES: CCOc1cc(C(F)(F)F)c2ccc3nc(-c4nnco4)cn3c2n1]

2-(2,4-bis(trifluoromethyl)imidazo[1′2′:1,6]pyrido[2,3-d]pyrimidin-8-yl)-1,3,4-oxadiazole free base has the following structure:

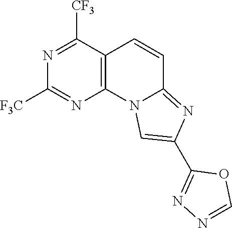 [SMILES: FC(F)(F)c1nc(C(F)(F)F)c2ccc3nc(-c4nnco4)cn3c2n1]

2-[2-ethyl-4-(trifluoromethyl)imidazo[1,2-a]1,8-naphthyridin-8-yl]-1,3,4-oxadiazole free base has the following structure:

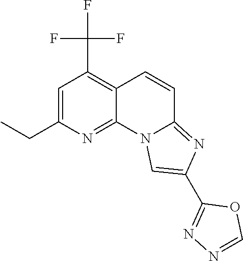 [SMILES: CCc1cc(C(F)(F)F)c2ccc3nc(-c4nnco4)cn3c2n1]

2-[2-phenyl-4-(trifluoromethyl)imidazo[1,2-a]1,8-naphthyridin-8-yl]-1,3,4-oxadiazole free base has the following structure:

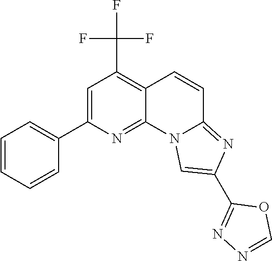 [SMILES: FC(F)(F)c1cc(-c2ccccc2)nc2c1ccc1nc(-c3nnco3)cn12]

2-[9-chloro-2,4-bis(trifluoromethyl)imidazo[1,2-a]1,8-naphthyridin-8-yl]-1,3,4-oxadiazole free base has the following structure:

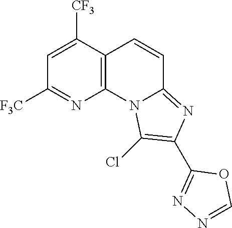 [SMILES: FC(F)(F)c1cc(C(F)(F)F)c2ccc3nc(-c4nnco4)c(Cl)n3c2n1]

2-[2-(difluoromethoxy)-4-(trifluoromethyl)imidazo[1,2-a]1,8-naphthyridin-8-yl]-1,3,4-oxadiazole free base has the following structure:

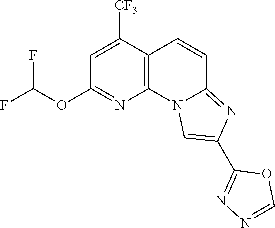 [SMILES: FC(F)Oc1cc(C(F)(F)F)c2ccc3nc(-c4nnco4)cn3c2n1]

2-[4-chloro-2-(propan-2-yl)imidazo[1,2-a]1,8-naphthyridin-8-yl]-1,3,4-oxadiazole free base has the following structure:

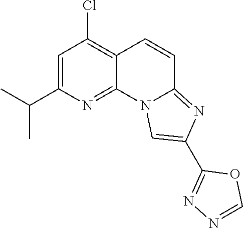 [SMILES: CC(C)c1cc(Cl)c2ccc3nc(-c4nnco4)cn3c2n1]

8-(furan-2-yl)-2,4-bis(trifluoromethyl)imidazo[1,2-a]1,8-naphthyridine free base has the following structure:

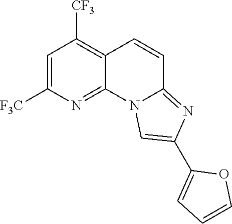 [SMILES: FC(F)(F)c1cc(C(F)(F)F)c2ccc3nc(-c4ccco4)cn3c2n1]

2-{2,4-dimethylimidazo[1,2-a]1,8-naphthyridin-8-yl}-1,3,4-oxadiazole free base has the following structure:

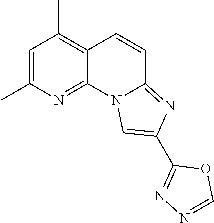 [SMILES: Cc1cc(C)c2ccc3nc(-c4nnco4)cn3c2n1]

2-[2,4-bis(trifluoromethyl)imidazo[1,2-a]1,8-naphthyridin-8-yl]-1,3-oxazole free base has the following structure:

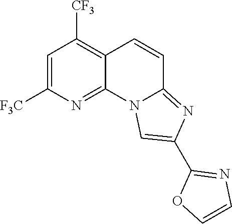 [SMILES: FC(F)(F)c1cc(C(F)(F)F)c2ccc3nc(-c4ncco4)cn3c2n1]

2-[2-chloro-4-(trifluoromethyl)imidazo[1,2-a]1,8-naphthyridin-8-yl]-1 ,3,4-oxadiazole free base has the following structure:

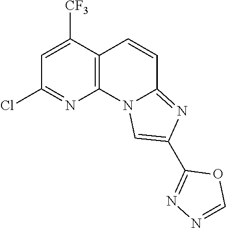 [SMILES: FC(F)(F)c1cc(Cl)nc2c1ccc1nc(-c3nnco3)cn12]

2-[2,4-bis(propan-2-yl)imidazo[1,2-a]1,8-naphthyridin-8-yl]-1,3,4-oxadiazole free base has the following structure:

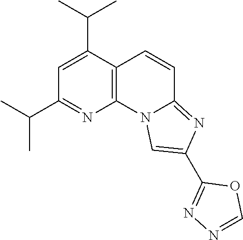 [SMILES: CC(C)c1cc(C(C)C)c2ccc3nc(-c4nnco4)cn3c2n1]

2-[4-phenyl-2-(propan-2-yl)imidazo[1,2-a]1,8-naphthyridin-8-yl]-1,3,4-oxadiazole free base has the following structure:

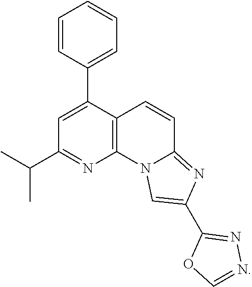 [SMILES: CC(C)c1cc(-c2ccccc2)c2ccc3nc(-c4nnco4)cn3c2n1]

The following examples and synthetic schemes serve to more fully describe the manner of making and using the above-described compounds. It is understood that these examples in no way serve to limit the scope of the invention, but rather are presented for illustrative purposes. In the examples and the synthetic schemes below, the following abbreviations have the following meanings. If an abbreviation is not defined, it has its generally accepted meaning.
aq. Aqueous
BHQ Black Hole Quencher fluorescent quencher for oligonucleotide primer for
FAM qPCR Fluorescein AMidite fluorescent dye for
NL oligonucleotide primer for qPCR Microliters
μM Micromolar
NMR nuclear magnetic resonance
boc tert-butoxycarbonyl
br Broad
Cbz Benzyloxycarbonyl
d Doublet
δ chemical shift
° C. degrees celcius
DCM Dichloromethane
dd doublet of doublets
DMEM Dulbeco's Modified Eagle's Medium
DMF N,N-dimethylformamide
DMSO Dimethylsulfoxide
EtOAc ethyl acetate
g Gram
h or hr Hours
HCV hepatitis C virus
HPLC high performance liquid chromatography
Hz Hertz
IU International Units
IC₅₀ inhibitory concentration at 50% inhibition
J coupling constant (given in Hz unless otherwise indicated)
m Multiplet
M Molar
M+H⁺ parent mass spectrum peak plus H⁺
mg Milligram
mL Milliliter
mM Millimolar
mmol Millimole
MS mass spectrum
nm Nanomolar
ppm parts per million
q.s. sufficient amount
s Singlet
sat. Saturated
t Triplet
TFA trifluoroacetic acid
   The compounds described herein may be prepared in accordance with the following synthetic schemes and Example.

### General Synthesis Schemes

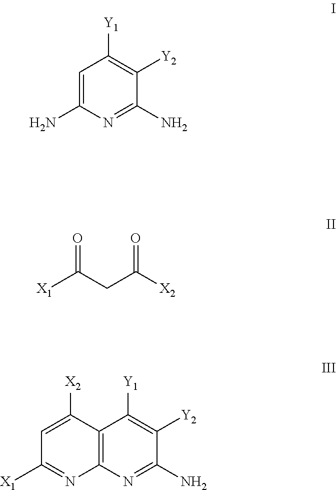

1,8-napthyridines of the general type III can be prepared from the corresponding 1,6-bisamino pyridines of general formula I and a corresponding diketone of general formula II. For example, those skilled in the art will recognize that treatment of I (Y₁=Y₂=H) with II (X₁=X₂=CF₃) in the presence of a suitable solvent (for example acetic acid) and heat (for example 80° C.) will give the corresponding napthyridine III (Y₁=Y₂=H; X₁=X₂=CF₃). Similarly, treatment of I (Y₁=Y₂=H) with II (X₁=OEt, X₂=CF₃) in the presence of solvent (diphenyl ether) and heat (for example 130° C. for 5 hours followed by 210° C. for 16 hours) affords III (X₁=OH, X₂=CF₃, Y₁=Y₂=H). Those skilled in the art will recognize this constitutes a general approach toward the preparation of molecules of general formula III of many different substitutions.

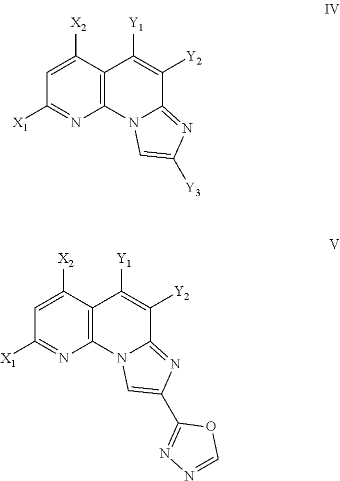

The corresponding 1,8-napthyridines of general formula III may be treated with an alkylating agent (for example a-bromopyruvate) in solvent (for example DMF) with heat (for example 80° C.) to afford tricyclic structures of general formula IV (where Y₃=CO₂Et if α-ethylbromopyruvate is used as an alkylating agent). Those skilled in the art will recognize alternate alkylating agents (preferably a-halo ketones, including, for example, α-bromoacetophenone or 2-bromo-1-(furan-2-yl)ethanone) may be employed in this transformation to afford compounds of formula IV where Y₃=phenyl or furyl respectively. Additionally, one skilled in the art will recognize when an alkylating agent is used to afford molecules of general formula IV with Y₃=CO₂Et, the ester functionality may be converted to any of a number of other structures (including, for example, oxazoles or oxadiazoles). For example, by treatment with hydrazine in solvent (for example ethanol) with heat (for example 80° C.) followed by subsequent exposure to a formate ester (for example trimethylorthoformate) with acid (for example p-toluenesulfonic acid) provides molecules of the general formula V. Alternatively, for molecules of general formula IV (Y₃=CO₂Et) may be readily converted to the corresponding aldehyde by treatement with a reducing agent (for example DIBALH) in solvent (for example toluene) with reduced temperature (for example −78° C.). Subsequent conversion to the corresponding oxazole (by treatment with the TOSMIC reagent, for example) can be readily accomplished using protocols well-known to those skilled in the art. Those skilled in the art will recognize an ester functionality may be transformed using standard conditions to numerous other heterocyclic rings.

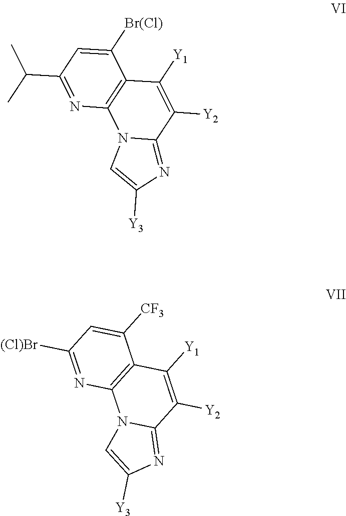

Those skilled in the art will recognize that molecules of general formula IV or V (wherein either X₁ or X₂ or both ═OH) may be converted to the corresponding halides (for example X₁ or X₂ or both ═Cl or Br) via treatment with a halogenating reagents (for example POCl₃ or POBr₃) in solvent (for example acetonitrile) with heat (for example 80° C.) to give, for example, molecules of general formula VI or VII. Aryl halides VI and VII may be transformed using well known chemistries (for example Suzuki, Stille, Negishi, or SNAR displacement chemistries) to afford molecules of the general formula IV or V wherein either X₁ or X₂ or both may be substituted with alkyl, aryl, amino, hydroxyl, or heteraryl functionalities. For example, treatment of molecules of general formula VI using Suzuki conditions including a vinyl boronic acid (for example cyclopentenyl boronic acid), a base (for example potassium carbonate) and a catalyst (for example PdCl₂(dppf)-CH₂Cl₂) in solvent (for example dioxane) followed by reduction of the corresponding olefin with a catalyst (for example palladium on carbon) in solvent (for example

THF) under an atmosphere of hydrogen can afford molecules of the general formula IV or V where X₂=cyclopentyl.

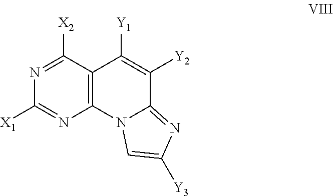

Those skilled in the art will further recognize additional core structures, for example molecules of general formula VIII can be prepared using analogous chemistries. For example treatment of compounds of general formula I with an electron deficient triazine (for example 2,4,6-tris(trifluoromethyl)-1,3,5-triazine) in solvent, followed by alkylation and derivatization in a manner analogous to that described above, affords molecules of general formula VIII. Once in hand, molecules of general formula VIII may be functionalized in a manner analogous to that described above for related core structures.

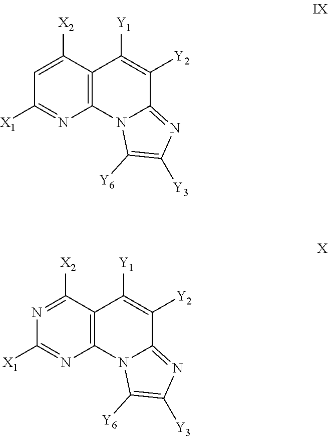

Direct functionalization of molecules of general formula IV and VIII to afford IX and X, respectively (for example Y₆=Cl or Br) can be accomplished via direct treatment of IV or VIII with a halogenating reagent (for example NCS or NBS) in solvent (for example DMF or chloroform). Those skilled in the art will recognize that for IX and X where Y₆=Br or Cl, a number of additional transformations are possible. For example, treatment of IX (Y₆=Br) under Negishi conditions including a catalyst (for example tetrakistriphenylphosphine palladium) and an organometalic reagent (for example dimethyl zinc) in a solvent (for example THF) with heat (for example 60° C.) will afford molecules of general structure IX wherein Y₆=Me.

### EXAMPLES

### Example 1

### 2-[2,4-bis(trifluoromethyl)imidazo[1,2-a]1,8-naphthyridin-8-yl]-1,3,4-oxadiazole

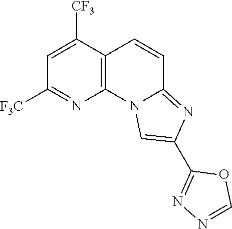 [SMILES: FC(F)(F)c1cc(C(F)(F)F)c2ccc3nc(-c4nnco4)cn3c2n1]

### Step A: 5,7-bis(trifluoromethyl)-1,8-naphthyridin-2-amine

A mixture of pyridine-2,6-diamine (12 g, 110 mmol) and 1,1,1,5,5,5-hexafluoropentane-2,4-dione (25.2 g, 121 mmol) dissolved in acetic acid (80 mL) was heated at 120° C. under nitrogen for 1 hour. After cooling to room temperature, the reaction mixture was concentrated and then diluted with ice water. The resulting solid was filtered and washed with water to give 5,7-bis(trifluoromethyl)-1,8-naphthyridin-2-amine (23.98 g, 85 mmol, 78% yield) as a grey solid. ES LC-MS m/z=282.10 (M+H)+.

### Step B: ethyl 2,4-bis(tritluoromethyl)imidazo[1,2-a][1,8]naphthyridine-8-carboxylate

To a solution of 20 g 5,7-bis(trifluoromethyl)-1,8-naphthyridin-2-amine in N,N-dimethylformamide (80 mL) was added ethyl 3-bromo-2-oxopropanoate (22.4 mL, 177 mmol) (2.5 eq) and the reaction mixture was heated at 68° C. under nitrogen for 3 h. The mixture was cooled room temperature, diluted with large quality of water and the resulting solid was filtered, and washed with water to give ethyl 2,4-bis(trifluoromethyl)imidazo[1,2-a][1,8]naphthyridine-8-carboxylate (13.55 g, 35.9 mmol, 32.7% yield) as a yellow brown solid, yield 50.5%. ES LC-MS m/z=378.20 (M+H)+.

### Step C: 2,4-bisarifluoromethAimidazo[1,2-a][1,8]naphthyridine-8-carbohydrazide

A solution of ethyl 2,4-bis(trifluoromethyl)imidazo[1,2-a][1,8]naphthyridine-8-carboxylate (25.5 g, 67.6 mmol) and hydrazine (42.4 mL, 1352 mmol) in ethanol (200 mL) was stirred at 65° C. for 2 hours. The mixture was cooled room temperature, and the precipitate was filtered off and washed with water to give 2,4-bis(trifluoromethyl)imidazo[1,2-a][1,8]naphthyridine-8-carbohydrazide (20.2 g, 55.6 mmol, 82% yield). ES LC-MS m/z=364.20 (M+H)+.

### Step D: 2-[2,4-bis(trifluoromethyl)imidazo[1,2-a]1,8-naphthyridin-8-yl]-1,3,4-oxadiazole

A solution of 2,4-bis(trifluoromethyl)imidazo[1,2-a][1,8]naphthyridine-8-carbohydrazide (19.5 g, 53.7 mmol) and tosic acid (5.11 g, 26.8 mmol) in trimethylorthoformate (5.93 ml, 53.7 mmol) was stirred with heating at 70° C. for 4 hours. The solution was cooled to room temperature and most of the solvent was evaporated. The resulting slurry was filtered and the filter cake was washed with water to give 2-[2,4-bis(trifluoromethyl)imidazo[1,2-a]1,8-naphthyridin-8-yl]-1,3,4-oxadiazole (12.4 g, 33.2 mmol, 61.9% yield). ¹H NMR (400 MHz, DMSO-d₆) δ ppm 8.00 (dd, 1 H) 8.14; (d, J=9.76 Hz, 1 H) 8.53; (s, 1 H) 9.23; (s, 1 H) 9.46; (s, 1 H);). ES LC-MS m/z=374.15 (M+H)+.

### Example 2

### 2-[2-cyclopentyl-4-(trifluoromethyl)imidazo[1,2-a]1,8-naphthyridin-8-yl]-1,3,4-oxadiazole

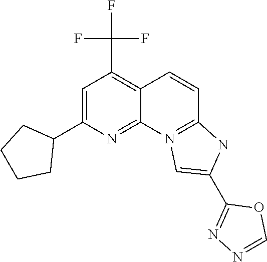

### Step A: 7-amino-4-(tritluoromethyl)-1,8-naphthyridin-2(1H)-one

A mixture of ethyl 4,4,4-trifluoro-3-oxobutanoate (14.2 g, 77 mmol) and 2,6-diaminopyridine (6 g, 55 mmol) in diphenyl ether (80 mL) was heated to 130° C. for 2 h, and then 190° C. for 18 h. The reaction was cooled to rt and diluted with hexanes, solids filtered and dried to afford the title compound (12.2 g, 97%). LC-MS: ESI (M+H)+m/z=230.13.

### Step B: ethyl 2-oxo-4-(tritluoromethyl)-1,2-dihydroimidazo[1,2-a]-1,8-naphthyridine-8-carboxylate

To a suspension of 7-amino-4-(trifluoromethyl)-1,8-naphthyridin-2(1H)-one (12.2 g, 53.2 mmol) in anhydrous DMF (180 mL) was added ethyl 3-bromo-2-oxopropanoate (11.4 g, 58.6 mmol) and the mixture heated to 60° C. for 18 h under nitrogen. The solvent was removed in vacuo and the residue partitioned between ethyl acetate and water. The aqueous layer was extracted with ethyl acetate and the combined organic layers dried (MgSO₄) and concentrated in vacuo. The residue was triturated in dichloromethane and the solids filtered and dried to afford the title compound (5.97 g, 34% yield). LC-MS: ESI (M+H)+m/z=326.19.

### Step C: 8-(1,3,4-oxadiazol-2-yl)-4-(trifluoromethyl)imidazo[1,2-a]1,8-naphthyridin-2(1H)-one

To a suspension of ethyl 2-oxo-4-(trifluoromethyl)-1,2-dihydroimidazo[1,2-a]-1,8-naphthyridine-8-carboxylate (2 g, 6.2 mmol) in ethanol was added hydrazine (3.9 g, 123 mmol) and the reaction heated to reflux for 18 h under nitrogen. The reaction was cooled to room temperature, and the solids were filtered and dried. The solids were suspended in triethyl orthoformate (25 mL), and p-toluenesulfonic acid monohydrate (0.59 g, 3.1 mmol) was added and the reaction heated to 85° C. for 2h. The reaction mixture was filtered without cooling and the solids dried to afford the title compound (1.48 g, 75% yield). LC-MS: ESI (M+H)+m/z=321.94.

### Step D: 2-chloro-8-(1,3,4-oxadiazol-2-yl)-4-(trifluoromethyl)imidazo[1,2-a]1,8-naphthyridine

A mixture of 8-(1,3,4-oxadiazol-2-yl)-4-(trifluoromethyl)imidazo[1,2-a]-1,8-naphthyridin-2(1H)-one (1.28 g. 4.0 mmol) and phosphorus oxytrichloride (13 mL) was heated to 100° C. under nitrogen for 1 h. The POCl3 was removed in vacuo and the residue stirred with water for 5 min and neutralized with potassium carbonate until the solution gave blue pH paper. The solution was extracted twice with dichloromethane and the organic layer dried (MgSO4) and concentrated in vacuo. The residue was triturated with ether and the solids filtered and dried to afford the title compound (774 mg, 57% yield). LC-MS: ESI (M+H)+m/z=340.12.

### Step E: 2-[2-cyclopentyl-4-(trifluoromethyl)imidazo[1,2-a]1,8-naphthyridin-8-yl]-1,3,4-oxadiazole

A mixture of 2-chloro-8-(1,3,4-oxadiazol-2-yl)-4-(trifluoromethyl)imidazo[1,2-a]-1,8-naphthyridine (85 mg, 0.25 mmol) and PdCl2(dppf)-CH2Cl2 (20 mg, 0.025 mmol) in anhydrous dioxane (2 mL) was degassed with nitrogen. To the solution was added cyclopentylzinc bromide as a 0.5 M solution in THF (0.6 mL) and the reaction heated to 80° C. in a sealed tube for 1 h, then 100° C. for 1 h. The reaction was treated with water and the resulting mixture partitioned between ethyl acetate and water. The organic layer was washed with brine, dried (MgSO4) and concentrated in vacuo. The residue was purified by silica gel chromatography eluting with 20-100% hexanes/ethyl acetate to afford the title compound (5 mg, 5% yield). LC-MS: ESI (M+H)+m/z=374.29. 1H NMR (400 MHz, DMSO-d₆) δ ppm 9.43; (s, 1 H), 9.13-9.29; (m, 1 H), 8.03; (s, 1 H), 7.79-7.95; (m, 2 H), 3.45-3.68; (m, 1 H), 2.15; (br. s., 2 H), 1.82-2.08; (m, 3 H), 1.60-1.81; (m, 2 H), 1.23; (br. s., 1 H).

### Example 3

### 2-[2-(propan-2-yl)-4-(trifluoromethyl)imidazoil,2-a]1,8-naphthyridin-8-yl.1-1,3,4-oxadiazole

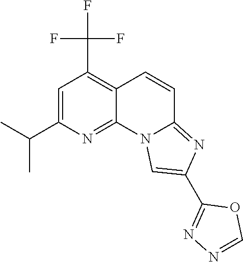 [SMILES: CC(C)c1cc(C(F)(F)F)c2ccc3nc(-c4nnco4)cn3c2n1]

Prepared from 2-chloro-8-(1,3,4-oxadiazol-2-yl)-4-(trifluoromethyl)imidazo[1,2-a]-1,8-naphthyridine in a manner similar as described in example 2, step E. LC-MS: ESI (M+H)⁺ m/z=348.25. 1H NMR (400 MHz, DMSO-d₆) δ ppm 9.43; (s, 1 H), 9.23; (s, 1 H), 8.05; (s, 1 H), 7.78-7.96; (m, 2 H), 3.37-3.48; (m, 1 H), 1.33-1.50; (m, 6 H).

### Example 4

### 5-[2,4-bis(trifluoromethyl)imidazo[1,2-a]1,8-naphthyridin-8-yl]-1,3-oxazole

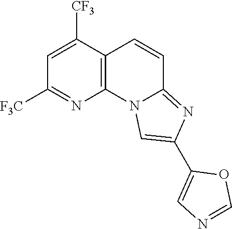 [SMILES: FC(F)(F)c1cc(C(F)(F)F)c2ccc3nc(-c4cnco4)cn3c2n1]

### Step A: 2,4-bisarifluoromethyl)imidazo[1,2-a][1,8]naphthyridine-8-carbaldehyde

To a solution of ethyl 2,4-bis(trifluoromethyl)imidazo[1,2-a][1,8]naphthyridine-8-carboxylate (500 mg, 1.325 mmol) in dichloromethane (15 mL) stirred under nitrogen at −78° C. was added DIBAL-H (1.0M solution) (3.98 mL, 3.98 mmol) dropwise over 30 minutes. After 2 hours at −78° C., the reaction was quenched with methanol at −78° C. Then the reaction mixture was allowed to warm to 0° C. and treated with a saturated solution of Rochelle's salt (100 mL). The resulting mixture was extracted with DCM (emulsion formed was filtered over Celite to remove white gummy precipitate). The combined extracts were concentrated under vacuum and the residue was purified via silica gel chromatography (0-5% MeOH/DCM) to give 2,4-bis(trifluoromethyl)imidazo[1,2-a][1,8]naphthyridine-8-carbaldehyde (293 mg, 0.835 mmol, 63.0 yield) as a light brown solid. ES LC-MS m/z=334.20 (M+H)⁺.

### Step B: 5-[2,4-bis(trifluoromethyl)imidazo[1,2-a]1,8-naphthyridin-8-yl]-1,3-oxazole

To a mixture of 2,4-bis(trifluoromethyl)imidazo[1,2-a][1,8]naphthyridine-8-carbaldehyde (100 mg, 0.300 mmol) and TOSMIC reagent (58.6 mg, 0.300 mmol) in methanol (4 mL) was added K₂CO₃ (41.5 mg, 0.300 mmol). The solution was refluxed for 2 hours, and the solvent was evaporated under reduced pressure. The residue was poured into ice water and extracted with DCM. The organic layer was washed consecutively with 1% HCl, followed by water, and concentrated to dryness. The crude material was purified via silica gel chromatography (0-5% MeOH/DCM) to give 5-[2,4-bis(trifluoromethyl)imidazo[1,2-a]1,8-naphthyridin-8-yl]-1,3-oxazole (84.1 mg, 0.215 mmol, 71.5% yield) as a yellow solid.: ¹H NMR (400 MHz, DMSO-d₆) δ ppm 7.80; (s, 1 H) 7.93; (dd, J=9.85, 1.85 Hz, 1 H) 8.08; (d, J=9.76 Hz, 1 H) 8.47; (s, 1 H) 8.57; (s, 1 H) 8.96; (s, 1 H); ES LC-MS m/z=373.22 (M+H)⁺.

### Example 5

### 2-[2-cyclopropyl-4-(trifluoromethyl)imidazo[1,2-a]1,8-naphthyridin-8-yl]-1,3,4-oxadiazole

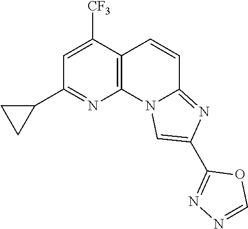 [SMILES: FC(F)(F)c1cc(C2CC2)nc2c1ccc1nc(-c3nnco3)cn12]

To a mixture of 2-(2-chloro-4-(trifluoromethyl)imidazo[1,2-a][1,8]naphthyridin-8-yl)-1,3,4-oxadiazole (34 mg, 0.100 mmol) and Pd(Ph₃P)₄ (11.57 mg, 10.01 μmol) dissolved in N,N-dimethylformamide (2 mL) was added cyclopropylzinc(II) bromide (0.400 mL, 0.200 mmol) dropwise. The reaction mixture was heated at 60° C. for 45 minutes under nitrogen, and the crude reaction mixture was purified via reverse phase HPLC to give 2-[2-cyclopropyl-4-(trifluoromethyl)imidazo[1,2-a]1,8-naphthyridin-8-yl]-1,3,4-oxadiazole (11.6 mg, 0.032 mmol, 31.9% yield) as a yellow solid. .¹H NMR (400 MHz, DMSO-d₆) δ: ppm 1.18-1.32; (m, 2 H) 1.31-1.41; (m, 2 H) 2.52-2.62; (m, 1 H) 7.84; (s, 2 H) 8.12; (s, 1 H) 9.17; (s,1 H) 9.42; (s,1 H); ES LC-MS m/z=346.24 (M+H)⁺.

### Example 6

### 2-[2-methyl-4-(trifluoromethyl)imidazo[1,2-a]1,8-naphthyridin-8-yl]-1,3,4-oxadiazole

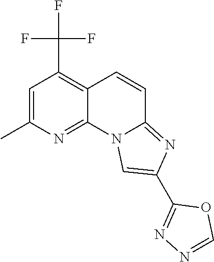 [SMILES: Cc1cc(C(F)(F)F)c2ccc3nc(-c4nnco4)cn3c2n1]

Prepared from 2-chloro-8-(1,3,4-oxadiazol-2-yl)-4-(trifluoromethyl)imidazo[1,2-a]-1,8-naphthyridine in a manner similar as described in example 2, step E. LC-MS: ESI (M+H)⁺ m/z=320.22. ¹H NMR (400 MHz, DMSO-d₆) δ ppm 9.42; (s, 1 H), 9.16; (s, 1 H), 8.06; (s, 1 H), 7.82-7.96; (m, 2 H), 2.84; (s, 3 H).

### Example 7

### 2-[9-methyl-2,4-bis(trifluoromethyl)imidazo[1,2-a]1,8-naphthyridin-8-yl]-1,3,4-oxadiazole

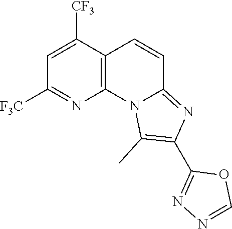 [SMILES: Cc1c(-c2nnco2)nc2ccc3c(C(F)(F)F)cc(C(F)(F)F)nc3n12]

### Step A: 2-(9-bromo-2,4-bis(trifluoromethyl)imidazo[1,2-a][1,8]naphthyridin-8-yl)-1,3,4-oxadiazole

A solution of 2-(2,4-bis(trifluoromethyl)imidazo[1,2-a][1,8]naphthyridin-8-yl)-1,3,4-oxadiazole (1.5 g, 4.02 mmol) and NBS (1.431 g, 8.04 mmol) in N,N-dimethylformamide (4 mL) was stirred with heating at 60° C. for 1 hour. Water was added and the precipitate was filtered off to give 2-(9-bromo-2,4-bis(trifluoromethyl)imidazo[1,2-a][1,8]naphthyridin-8-yl)-1,3,4-oxadiazole (1.69 g, 3.55 mmol, 88% yield). ES LC-MS m/z=452.13 (Br⁷⁹, M+H)⁺, ES LC-MS m/z=454.10 (Br⁸¹, M+H)⁺.

### Step B: 2-[9-methyl-2,4-bis(trifluoromethyl)imidazo[1,2-a]1,8-naphthyridin-8-yl]-1,3,4-oxadiazole

A solution of 2-(9-bromo-2,4-bis(trifluoromethyl)imidazo[1,2-a][1,8]naphthyridin-8-yl)-1,3,4-oxadiazole (100 mg, 0.221 mmol), 2,4,6-trimethyl-1,3,5,2,4,6-trioxatriborinane (278 mg, 0.221 mmol), PdCl₂(dppf)-CH₂Cl₂ adduct (18.06 mg, 0.022 mmol) and sodium carbonate (0.332 mL, 0.664 mmol, 1.0 M solution) in N,N-dimethylacetamide (5.0 mL) was heated at 60° C. for 1 hour. The crude reaction mixture was purified via reverse phase HPLC to give 2-[9-methyl-2,4-bis(trifluoromethyl)imidazo[1,2-a]1,8-naphthyridin-8-yl]-1,3,4-oxadiazole (7.2 mg, 0.018 mmol, 7.99% yield): ¹H NMR (400 MHz, DMSO-d₆) δ ppm 3.35; (s, 3 H) 7.91; (d, J=9.76 Hz, 1 H) 8.08; (d, J=9.76 Hz, 1 H) 8.50; (s, 1 H) 9.43; (s, 1 H); ES LC-MS m/z=388.24 (M+H)⁺.

### Example 8

### 2-[2-ethoxy-4-(trifluoromethyl)imidazo[1,2-a]1,8-naphthyridin-8-yl]-1,3,4-oxadiazole

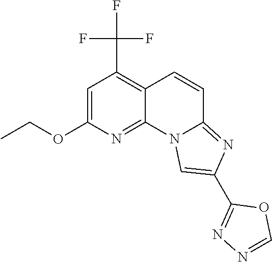 [SMILES: CCOc1cc(C(F)(F)F)c2ccc3nc(-c4nnco4)cn3c2n1]

To a solution of 2-chloro-8-(1,3,4-oxadiazol-2-yl)-4-(trifluoromethyl)imidazo[1,2-a]-1,8-naphthyridine (example 2, step D) (50 mg, 0.15 mmol) in ethanol (1 mL) was added sodium ethoxide (21 wt % in ethanol, 0.07 mL, 0.18 mmol) and the reaction stirred at room temperature for 45 min and then at 50° C. for 30 min. The reaction was cooled to room temperature, poured into ethyl acetate and washed with water, dried (MgSO₄) and concentrated in vacuo. The residue was purified by silica gel chromatography eluting with 50-100% hexanes/ethyl acetate to afford the title compound (19 mg, 31% yield). LC-MS: ESI (M+H)⁺ m/z=349.83. ¹H NMR (400 MHz, DMSO-d₆) δ ppm 9.42 (s, 1 H), 9.21; (s, 1 H), 7.72-7.90; (m, 2 H), 7.56; (s, 1 H), 4.71; (q, J=7.0 Hz, 2 H), 1.46; (t, J=7.0 Hz, 3 H).

### Example 9

### 2-(2,4-bis(trifluoromethyl)imidazo[1′2′:1,6]pyrido[2,3-d]pyrfinidin-8-yl)-1,3,4-oxadiazole

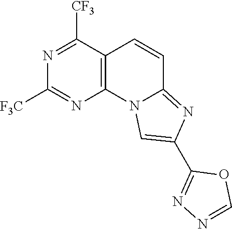 [SMILES: FC(F)(F)c1nc(C(F)(F)F)c2ccc3nc(-c4nnco4)cn3c2n1]

### Step A: 2,4-bis(trifluoromethyl)pyrido[2,3-d]pyrimidin-7-amine

A solution of pyridine-2,6-diamine (1.5 g, 13.75 mmol) in AcOH (64.8 ml) was cooled to 0 deg and treated by the drop wise addition of 2,4,6-tris(trifluoromethyl)-1,3,5-triazine (3.89 ml, 13.75 mmol). The bath was removed and the reaction was heated to 80° C. overnight. After cooling to room temperature, the solvents were removed under reduced pressure and the residue was taken up in DCM and basified with 1N NaOH. The combined organics were washed with saturated NaHCO₃ (3×), brine, dried over Na₂SO₄, filtered, and concentrated to give 2,4-bis(trifluoromethyl)pyrido[2,3-d]pyrimidin-7-amine (3.77 g, 13.36 mmol, 97% yield) as a red solid. ES LC-MS m/z=283.11 (M+H)⁺.

### Step B: ethyl 2,4-bis(tritluoromethyl)imidazo[1′,2′:1,6]pyrido[2,3-d]pyrimidine-8-carboxylate

A solution of 2,4-bis(trifluoromethyl)pyrido[2,3-d]pyrimidin-7-amine (2.0 g, 7.09 mmol) in DMF (33.2 ml) was treated with ethyl borompyruvate (2.230 ml, 17.72 mmol). The reaction was heated to 80° C. overnight. The black reaction was concentrated under reduced pressure to remove most of the DMF. The residue was diluted with H₂O and the solids were filtered to give ethyl 2,4-bis(trifluoromethyl)imidazo[1′,2′:1 ,6]pyrido[2,3-d]pyrimidine-8-carboxylate (2.45 g, 6.48 mmol, 91% yield) as a brown solid. ES LC-MS m/z=379.14 (M+H)⁺.

### Step C: 2,4-bis(trifluoromethyl)imidazo[1′,2′:1,6]pyrido[2,3-d]pyrimidine-8-carbohydrazide

A solution of ethyl 2,4-bis(trifluoromethyl)imidazo[1′,2′:1,6]pyrido[2,3-d]pyrimidine-8-carboxylate (0.5 g, 1.322 mmol) and hydrazine (0.830 ml, 26.4 mmol) in EtOH (5.78 ml) was heated to reflux for 30 minutes The reaction was concentrated under reduced pressure to give 2,4-bis(trifluoromethyl)imidazo[1′,2′:1,6]pyrido[2,3-d]pyrimidine-8-carbohydrazide (0.481 g, 1.321 mmol, 100% yield) as a dark red/brown oil. ES LC-MS m/z=365.1 (M+H)⁺.

### Step D: 2-(2,4-bis(tritluoromethyl)imidazo[1′,2′:1,6]pyrido[2,3-d]pyrimidin-8-yl)-1,3,4-oxadiazole

A solution of 2,4-bis(trifluoromethyl)imidazo[1′,2′:1,6]pyrido[2,3-d]pyrimidine-8-carbohydrazide (0.481 g, 1.321 mmol), TsOH (0.100 g, 0.528 mmol), and triethyl orthoformate (8.80 ml, 52.8 mmol) was heated at 80 ° C. under nitrogen overnight. After cooling to room temperature, the solvents were removed under reduced pressure and the residue was treated by water. The solution was extracted with EtOAc. The combined extracts were washed with brine, dried over Na₂SO₄, filtered, and concentrated. The residue was taken up in DMF and purified by reverse phase chromatography (10-90% ACN/H₂O+formic acid), then lyophilized to give 2-(2,4-bis(trifluoromethyl)imidazo[1′,2′:1,6]pyrido[2,3-d]pyrimidin-8-yl)-1,3,4-oxadiazole (0.0436 g, 0.117 mmol, 8.82% yield) as a solid. ¹H NMR (400 MHz, DMSO-d₆) 6 ppm 9.51 (s, 3 H), 9.39; (s, 1 H), 8.07-8.28; (m, 2 H), ES LC-MS m/z=375.2 (M+H)+.

### Example 10

### 2-[2-ethyl-4-(trifluoromethyl)imidazo[1,2-a]1,8-naphthyridin-8-yl]-1,3,4-oxadiazole

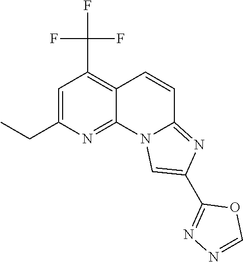 [SMILES: CCc1cc(C(F)(F)F)c2ccc3nc(-c4nnco4)cn3c2n1]

Prepared from 2-chloro-8-(1,3,4-oxadiazol-2-yl)-4-(trifluoromethyl)imidazo[1,2-a]-1,8-naphthyridine in a manner similar as described in example 2, step E. LC-MS: ESI (M+H)⁺ m/z=334.18. ¹H NMR (400 MHz, DMSO-d₆) δ ppm 9.43 (s, 1 H), 9.20; (s, 1 H), 8.05; (s, 1 H), 7.79-7.96; (m, 2 H), 3.13; (q, J=7.4 Hz, 2 H), 1.43; (t, J=7.5 Hz, 3 H).

### Example 11

### 2-[2-phenyl-4-(trifluoromethyl)imidazoi[1,2-a]1,8-naphthyridin-8-yl]-1,3,4-oxadiazole

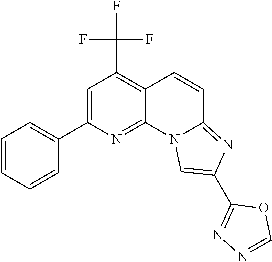 [SMILES: FC(F)(F)c1cc(-c2ccccc2)nc2c1ccc1nc(-c3nnco3)cn12]

A solution of 2-chloro-8-(1,3,4-oxadiazol-2-yl)-4-(trifluoromethyl)imidazo[1,2-a]-1,8-naphthyridine (50 mg, 0.15 mmol), PdCl₂ (dppf)-CH₂Cl₂ (12 mg, 0.015 mmol), phenylboronic acid (21 mg, 0.18 mmol) and potassium acetate (58 mg, 0.59 mmol) in dioxane (1.5 mL) was degassed with nitrogen and heated to 100° C. in a sealed tube for 1 h. The reaction was cooled to room temperature, poured into ethyl acetate and washed with water. The organic layer was concentrated to half volume, and the mixture filtered and solids dried to afford the title compound (42 mg, 70% yield). LC-MS: ESI (M+H)⁺ m/z=382.11. ¹H NMR (400 MHz, DMSO-d₆) δ ppm 9.57 (s, 1 H), 9.45; (s, 1 H), 8.49-8.77; (m, 3 H), 7.86-8.02; (m, 2 H), 7.49-7.80; (m, 3 H).

### Example 12

### 2-[9-chloro-2,4-bis(trifluoromethyl)imidazo[1,2-a]1,8-naphthyridin-8-yl]-1,3,4-oxadiazole

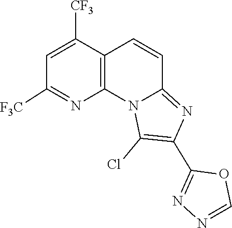 [SMILES: FC(F)(F)c1cc(C(F)(F)F)c2ccc3nc(-c4nnco4)c(Cl)n3c2n1]

A solution of 2-(2,4-bis(trifluoromethyl)imidazo[1,2-a][1,8]naphthyridin-8-yl)-1,3,4-oxadiazole (165 mg, 0.442 mmol) and 1-chloropyrrolidine-2,5-dione (236 mg, 1.768 mmol) in N,N-dimethylformamide (4 mL) was stirred at 60° C. for 2 hours. Water was added and the precipitate was filtered off to give 2-[9-chloro-2,4-bis(trifluoromethyl)imidazo[1,2-a]1,8-naphthyridin-8-yl]-1,3,4-oxadiazole (145 mg, 0.338 mmol, 76% yield). ¹H NMR (400 MHz, DMSO-d₆) δ ppm 7.99 (dd, 1 H) 8.11; (d, J=9.87 Hz, 1 H) 8.55; (s, 1 H) 9.50; (s, 1 H); ES LC-MS m/z=408.24 (M+H)⁺.

### Example 13

### 2-[2-(difluoromethoxy)-4-(trifluoromethyl)imidazo[1,2-a]1,8-naphthyridin-8-yl]-1,3,4-oxadiazole

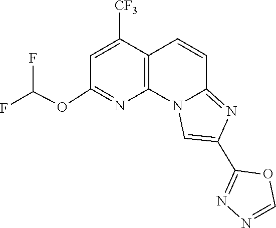 [SMILES: FC(F)Oc1cc(C(F)(F)F)c2ccc3nc(-c4nnco4)cn3c2n1]

A mixture of 8-(1,3,4-oxadiazol-2-yl)-4-(trifluoromethyl)imidazo[1,2-a][1,8]naphthyridin-2-ol (50 mg, 0.156 mmol), sodium 2-chloro-2,2-difluoroacetate (59.3 mg, 0.389 mmol) and Cs₂CO₃ (71.0 mg, 0.218 mmol) were dissolved in N,N-dimethylformamide (2 mL) was heated at 90° C. under nitrogen for 2 hours. The reaction mixture was purified via reverse phase HPLC to give 2-[2-(difluoromethoxy)-4-(trifluoromethyl)imidazo[1,2-a]1,8-naphthyridin-8-yl]-1,3,4-oxadiazole (22.2 mg, 0.057 mmol, 36.5% yield) as a light yellow solid. ¹H NMR (400 MHz, DMSO-d₆) δ: ppm 7.89-7.93; (m, 3 H) 8.45; (t, 1 H) 9.46; (s, 1 H) 9.52; (s, 1 H); ES LC-MS m/z=372.23 (M+H)⁺.

### Example 14

### 2-[4-chloro-2-(propan-2-yl)imidazo[1,2-a]1,8-naphthyridin-8-yl]-1,3,4-oxadiazole

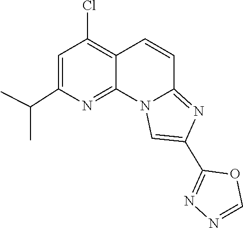 [SMILES: CC(C)c1cc(Cl)c2ccc3nc(-c4nnco4)cn3c2n1]

### Step A: 7-amino-2-(1-methylethyl)-1,8-naphthyridin-4(1H)-one

A solution of pyridine-2,6-diamine (5 g, 45.8 mmol) and ethyl 4-methyl-3-oxopentanoate (11.09 mL, 68.7 mmol) in diphenyl ether (50 mL) was maintained at 150° C. overnight and then warmed to 250° C. for another 24 hours. The mixture was cooled to room temperature and product allowed to crystallize out over 5 hours. The supernatant was poured off and the solids were triturated with DCM/MeOH and the solids collected via vacuum filtration to afford 7-amino-2-isopropyl-1,8-naphthyridin-4(1H)-one (3.3 g, 16.24 mmol, 35.4% yield) as a yellow solid. LC-MS: ESI (M+H)⁺ m/z=222.45.

### Step B: ethyl 2-(1-methylethyl)-4-oxo-1,4-dihydroimidazo[1,2-a]-1,8-naphthyridine-8-carboxylate

To a solution of 7-amino-2-(1-methylethyl)-1,8-naphthyridin-4(1H)-one (2.8 g, 13.8 mmol) in anhydrous DMF (40 mL) was added ethyl 3-bromo-2-oxopropanoate (4.0 g, 20.7 mmol) and the reaction stirred at 60° C. for 18 h. The reaction was cooled to room temperature and poured into ethyl acetate, washed with water, brine, dried (MgSO₄) and concentrated in vacuo. The residue was triturated in ether and filtered, the solids dried. The filtrate was concentrated in vacuo and the residue purified by silica gel chromatography eluting with 0-10% ethyl acetate/methanol. The eluent was combined with the filtered solids to afford the title compound (800 mg, 19% yield). LC-MS: ESI (M+H)⁺ m/z=299.82.

### Step C: 2-(1-methylethyl)-8-(1,3,4-oxadiazol-2-yl)imidazo[1,2-a]-1,8-naphthyridin-4(1H)-one

To a solution of ethyl 2-(1-methylethyl)-4-oxo-1,4-dihydroimidazo[1,2-a]-1,8-naphthyridine-8-carboxylate (922 mg, 3.1 mmol) in ethanol (25 mL) was added hydrazine (1.9 mL, 61.6 mmol) and the reaction heated to 85° C. overnight. The reaction was cooled to room temperature, the solvent removed in vacuo and the residue dried. To the residue was added triethyl orthoformate (20 mL) and p-toluenesulfonic acid monohydrate (586 mg, 3.1 mmol) and the reaction heated to 110° C. for 1 h. The reaction was cooled to room temperature, poured into ethyl acetate, washed with saturated sodium bicarbonate solution, and dried (MgSO₄) and concentrated in vacuo. The residue was triturated in ether and solids were filtered and dried to afford the title compound (175 mg, 19% yield). LC-MS: ESI (M+H)⁺ m/z=296.24.

### Step D: 2-[4-chloro-2-(propan-2-yl)imidazo[1,2-a]1,8-naphthyridin-8-yl]-1,3,4-oxadiazole

A mixture of 2-(1-methylethyl)-8-(1,3,4-oxadiazol-2-yl)imidazo[1,2-a]-1,8-naphthyridin-4(1H)-one (175 mg, 0.59 mmol) and phosphorus oxytrichloride (4 mL) was heated to 100° C. for 30 min. The reaction was cooled to room temperature and the volatiles removed in vacuo. The residue was stirred with water for 10 min and neutralized with potassium carbonate. The solution was extracted twice with dichloromethane and the organic layer dried (MgSO₄) and concentrated in vacuo . The residue was purified by silica gel chromatography eluting with 50-100% hexanes/ethyl acetate to afford the title compound (54 mg, 29% yield). LC-MS: ESI (M+H)⁺ m/z=314.25. ¹H NMR (400 MHz, DMSO-d₆) δ ppm 9.41; (s, 1 H), 9.14 (s, 1 H), 7.93-8.04; (m, 1 H), 7.86-7.93; (m, 1 H), 7.83; (d, J=9.8 Hz, 1 H), 3.21-3.31; (m, 1 H), 1.27-1.46; (m, 6 H).

### Example 15

### 8-(furan-2-yl)-2,4-bis(trifluoromethyl)imidazo[1,2-a]1,8-naphthyridine

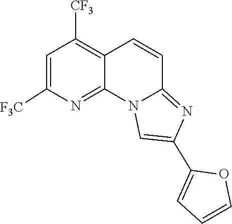 [SMILES: FC(F)(F)c1cc(C(F)(F)F)c2ccc3nc(-c4ccco4)cn3c2n1]

### Step A: 5,7-bis(trifluoromethyl)-1,8-naphthyridin-2-amine

A mixture of pyridine-2,6-diamine (10 g, 91 mmol), 1,1,1,5,5,5-hexafluoropentane-2,4-dione (19 g, 91 mmol) in H₃PO₄ (100 mL) was stirred at 95° C. overnight. After cooling to room temperature, the mixture was poured into ice/water mixture. The pH of the aqueous phase was adjusted to 7 with the addition of ammonium hydroxide. The solid formed was collected by vacuum filtration, washed with water, and dried under reduced pressure. The crude product was recrystallized in EtOH to provide 5,7-bis(trifluoromethyl)-1,8-naphthyridin-2-amine (8 g, 28 mmol, 30% of yield) as a green solid: ES LC-MS m/z=282 (M+H)⁺.

### Step B: 8-(furan-2-yl)-2,4-bis(trifluoromethyl)imidazo[1,2-a]1,8-naphthyridine

A mixture of 5,7-bis(trifluoromethyl)-1,8-naphthyridin-2-amine (100 mg, 0.356 mmol) and 2-bromo-1-(furan-2-yl)ethanone (88 mg, 0.427 mmol) was refluxed in EtOH (5 mL) overnight. The mixture was cooled to room temperature and EtOH was removed under reduced pressure. The residue was taken up with EtOAc (15 mL), washed with saturated NaHCO₃ (10 mL). The organic phase was dried over Na₂SO₄, filtered and concentrated. The residue was purified with column chromatography (silica gel, 0-10% of EtOAc in petroleum ether) to obtain 8-(furan-2-yl)-2,4-bis(trifluoromethyl)imidazo[1,2-]1,8-naphthyridine (50 mg, 0.13 mmol, 38% of yield) as a yellow solid: ¹H NMR (300 MHz, CDCl₃) δ ppm 8.79; (s, 1 H), 8.11; (s, 1 H), 7.98-7.87; (m, 2H), 7.58; (s, 1H), 7.02; (d, 1H), 6.59; (d, 1H); ES LC-MS m/z=372.0 (M+H)⁺.

### Example 16

### 2-{2,4-dimethylimidazo[1,2-a]1,8-naphthyridin-8-yl}-1,3,4-oxadiazole

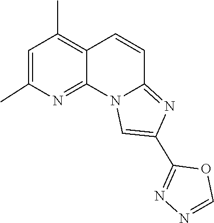 [SMILES: Cc1cc(C)c2ccc3nc(-c4nnco4)cn3c2n1]

### Step A: 5,7-dimethyl-1,8-naphthyridin-2-amine

A mixture of pyridine-2,6-diamine (2 g, 18.3 mmol), pentane-2,4-dione (1.83, 18.3 mmol) and H₂SO₄ (0.25 mL) in glacial acetic acid (10 mL) was refluxed for 8 hours. After cooling to room temperature, the mixture was poured into a mixture of ice/water. The pH of the aqueous phase was adjusted to 7 with the addition of ammonium hydroxide. The brown solid formed was collected with filtration, washed with water, dried and recrystallized in EtOH to provide 5,7-dimethyl-1,8-naphthyridin-2-amine (1 g, 5.7 mmol, 32%) as a brown solid: ¹H NMR (300 MHz, DMSO-d₆) δ ppm 8.04; (d, 1H), 6.91; (s, 1H), 6.74; (d, 1H), 6.59; (s, br, 2H), 2.49; (s, 3H), 2.48; (s, 3H); ES LC-MS m/z=174.0 (M+H)⁺.

### Step B: ethyl 2,4-dimethylimidazo[1,2-a][1,8]naphthyridine-8-carboxylate

A mixture of 5,7-dimethyl-1,8-naphthyridin-2-amine (900 mg, 5.2 mmol) and ethyl 3-bromo-2-oxopropanoate (1.15 g, 5.7 mmol) was refluxed in EtOH (10 mL) under nitrogen overnight. After cooling to room temperature, the mixture was concentrated and the residue was purified by silica gel chromatography (silica gel, 20% to 50% of EtOAc/petroleum ether) to provide ethyl 2,4-dimethylimidazo[1,2-a][1,8]naphthyridine-8-carboxylate (420 mg, 1.56 mmol, 30% of yield) as a yellow solid: ES LC-MS m/z=270.0 (M+H)⁺.

### Step C: 2,4-dimethylimidazo[1,2-a][1,8]naphthyridine-8-carbohydrazide

To a solution of ethyl 2,4-dimethylimidazo[1,2-a][1,8]naphthyridine-8-carboxylate (420 mg, 1.56 mmol) in EtOH (5 mL) was added hydrazine hydrate (780 mg, 15.6 mmol) at 0° C. The mixture was stirred at room temperature overnight. The yellow solid formed was collected by vacuum filtration, washed with EtOH and dried under reduced pressure to provide 2,4-dimethylimidazo[1,2-a][1,8]naphthyridine-8-carbohydrazide (300 mg, 1.17 mmol, 75% of yield) as yellow solid which was used in the next step without further purification. ES LC-MS m/z=256.1 (M+H)⁺.

### Step D: 2-{2,4-dimethylimidazo[1,2-a]1,8-naphthyridin-8-yl}-1,3,4-oxadiazole

A mixture of 2,4-dimethylimidazo[1,2-a][1,8]naphthyridine-8-carbohydrazide (200 mg, 0.78 mmol) and trimethyl orthoformate (166 mg, 1.57 mmol) was refluxed in EtOH (5 mL) overnight. After cooling to room temperature, the mixture was concentrated in vacuo. The residue was recrystallized in EtOH to provide 2-{2,4-dimethylimidazo[1,2-a]1,8-naphthyridin-8-yl}-1,3,4-oxadiazole (60 mg, 0.22 mmol, 29% of yield) as a light yellow solid: 1H NMR (300 MHz, CD₃OD) 6 ppm 9.10-9.08; (m, 2H), 7.96; (d, 1H), 7.54; (d, 1H), 7.36; (s, 1H), 2.68; (s, 6H). ES LC-MS m/z=266.1 (M+H)⁺.

### Example 17

### 2-[2,4-bis(trifluoromethyl)imidazo[1,2-a]1,8-naphthyridin-8-yl]-1,3-oxazole

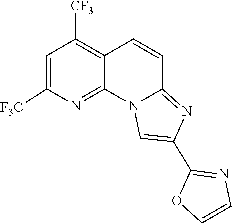 [SMILES: FC(F)(F)c1cc(C(F)(F)F)c2ccc3nc(-c4ncco4)cn3c2n1]

### Step A: ethyl 2,4-bis(tritluoromethyl)imidazo[1,2-a][1,8]naphthyridine-8-carboxylate

A mixture of 5,7-bis(trifluoromethyl)-1,8-naphthyridin-2-amine (1.5 g, 5.34 mmol) and ethyl 3-bromo-2-oxopropanoate (1.25 g, 6.4 mmol) was refluxed in EtOH (15 mL) for 4 hours. After cooling down to room temperature, the yellow solid was collected via vacuum filtration and washed with EtOH to afford ethyl 2,4-bis(trifluoromethyl)imidazo[1,2-a][1,8]naphthyridine-8-carboxylate (745 mg, 1.97 mmol, 37%) as yellow solid: ¹H NMR (300 MHz, CDCl₃) δ ppm 9.15 (s, 1H), 8.14; (s, 1H), 7.94-7.92; (m, 2H), 4.52; (q, 2H), 1.48; (t, 3H); ES LC-MS m/z=378.1 (M+H)⁺.

### Step B: 2,4-bisarifluoromethAimidazo[1,2-a][1,8]naphthyridine-8-carboxylic acid

To a solution of ethyl 2,4-bis(trifluoromethyl)imidazo[1,2-a][1,8]naphthyridine-8-carboxylate (400 mg, 1.06 mmol) in THF (15 mL) and water (15 mL) was added lithium hydroxide monohydrate (223 mg, 5.31 mmol). The mixture was stirred at room temperature for 1 hour. THF was removed under reduced pressure. The aqueous layer was acidified to pH 2-3 with the addition of 1M HCl, extracted with EtOAc (20 mL×2). The combined organic layer was washed with brine (50 mL), dried over Na₂SO₄, filtered and concentrated. The crude 2,4-bis(trifluoromethyl)imidazo[1,2-a][1,8]naphthyridine-8-carboxylic acid (320 mg, 0.92 mmol, 86% of crude yield) was used in the next step without further purification.

### Step C: N-(2,2-dimethoxyethyl)-2,4-bisarifluoromethAimidazo[1,2-a][1,8]naphthyridine-8-carboxamide

To a solution of 2,4-bis(trifluoromethyl)imidazo [1,2-a][1,8]naphthyridine-8-carboxylic acid (220 mg, 0.64 mmol) in DMF (20 mL) was added DIPEA (177 mg, 1.32 mmol), TBTU (205 mg, 0.64 mmol) and 2,2-dimethoxyethanamine (67 mg, 0.64 mmol). The resulting mixture was stirred at room temperature overnight. Water was added and the aqueous phase was extracted with EtOAc (50 mL×2). The combined organic phase s were washed with brine, dried over Na₂SO₄, filtered and concentrated to give a residue. The crude product was purified on column chromatography (20% of EtOAc/petroleum ether) to give N-(2,2-dimethoxyethyl)-2,4-bis(trifluoromethyl)imidazo[1,2-a][1,8]naphthyridine-8-carboxamide (220 mg, 80%) as a white solid. ES LC-MS m/z=436.1 (M+H)⁺.

### Step D: N-(2-oxoethyl)-2,4-bis(trifluoromethyl)imidazo[1,2-a][1,8]naphthyridine-8-carboxamide

To a solution of N-(2,2-dimethoxyethyl)-2,4-bis(trifluoromethyl)imidazo[1,2-a][1,8]naphthyridine-8-carboxamide (200 mg, 0.46 mmol) in DCM (20 mL) was added trifluoroacetic acid (262.2 mg, 2.3 mmol) at room temperature. The mixture was stirred at r.t. for 2 hours. The solution was washed with saturated NaHCO₃. The aqueous phase was extracted with EtOAc (10 mL×2). The combined organic phase was dried over Na₂SO₄, filtered and concentrated to provide N-(2-oxoethyl)-2,4-bis(trifluoromethyl)imidazo[1,2-a][1,8]naphthyridine-8-carboxamide (120 mg, 0.31 mmol, 67% of yield) which was used in the next step without further purification.

### Step E: 2-[2,4-bis(trifluoromethyl)imidazo[1,2-a]1,8-naphthyridin-8-yl]-1,3-oxazole

To a solution of N-(2-oxoethyl)-2,4-bis(trifluoromethyl)imidazo[1,2-a][1,8]naphthyridine-8-carboxamide (120 mg, 0.3 mmol) in DCM (20 mL) was added perchloroethane (141 mg, 0.6 mmol), PPh₃ (157.2 mg, 0.6 mmol) and Et₃N (151.5 mg, 1.5 mmol) at room temperature. The resulting mixture was stirred at r.t. overnight. The solvent was removed under vacuum and the residue was purified with column chromatography (silica gel, 20%-50% of EtOAc/petroleum ether) to provide 2-[2,4-bis(trifluoromethyl)imidazo[1,2-a]1,8-naphthyridin-8-yl]-1,3-oxazole (40 mg, 0.09 mmol, 35% of yield): ¹H NMR (300 MHz, CD₃OD) δ ppm 9.15; (s, 1H), 8.35; (s, 1H), 8.09-8.05; (m, 2H), 7.99; (m, 1 H), 7.40; (d, 1 H); ES LC-MS m/z=372.0 (M+H)⁺.

### Example 18

### 2-[2-chloro-4-(trifluoromethyl)imidazo[1,2-a]1,8-naphthyridin-8-yl]-1,3,4-oxadiazole

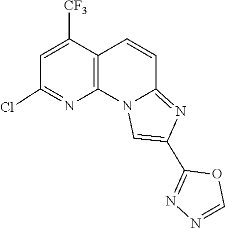 [SMILES: FC(F)(F)c1cc(Cl)nc2c1ccc1nc(-c3nnco3)cn12]

### Step A: 7-amino-4-(tritluoromethyl)-1,8-naphthyridin-2-ol

A mixture of pyridine-2,6-diamine (500 mg, 4.58 mmol) and ethyl 4,4,4-trifluoro-3-oxobutanoate (886 mg, 4.81 mmol) was heated until pyridine-2,6-diamine was completely dissolved. The mixture was cooled to 0° C. and concentrated H₂SO₄ (8 mL, 150 mmol) was added dropwise. The reaction mixture was then allowed to stand for 12 hours at 60° C., was poured into crushed ice and basified with 20% NaOH(aq) solution. The precipitate was filtered and washed with water to give (866 mg, Yield 82.9%) 7-amino-4-(trifluoromethyl)-1,8-naphthyridin-2-ol was afforded as a yellow solid. ES LC-MS m/z=230.02 (M+H)⁺.

### Step B: methyl 2-hydroxy-4-(trifluoromethyl)imidazo[1,2-a][1,8]naphthyridine-8-carboxylate

A mixture of 7-amino-4-(trifluoromethyl)-1,8-naphthyridin-2-ol (1 g, 4.36 mmol) and methyl 3-bromo-2-oxopropanoate (1.185 g, 6.55 mmol) in N,N-dimethylformamide (10 mL) was heated at 60° C. for 8 hours under nitrogen. After cooling to room temperature, the reaction mixture was diluted with water and the filtrate filtered off and washed with water to give 560 mg (yield 41.2%) methyl 2-hydroxy-4-(trifluoromethyl)imidazo[1,2-a][1,8]naphthyridine-8-carboxylate was afforded as a yellow solid. ES LC-MS m/z=326.03 (M+H)⁺.

### Step C: 2-hydroxy-4-(trifluoromethyl)imidazo[1,2-a][1,8]naphthyridine-8-carbohydrazide

To a solution of methyl 2-hydroxy-4-(trifluoromethyl)imidazo[1,2-a][1,8]naphthyridine-8-carboxylate (305 mg) dissolved in ethanol (8 mL) was added 20eq hydrazine (640 μl, 20.39 mmol) and the reaction mixture was refluxed for 4 hours under nitrogen. The mixture was cooled to room temperature and concentrated to dryness in vacuum to give 2-hydroxy-4-(trifluoromethyl)imidazo[1,2-a][1,8]naphthyridine-8-carbohydrazide (228 mg) as a yellow solid. ES LC-MS m/z=312.09 (M+H)⁺.

### Step D: 8-(1,3,4-oxadiazol-2-yl)-4-(trifluoromethyl)imidazo[1,2-a][1,8]naphthyridin-2-ol

A mixture of 100 mg 2-hydroxy-4-(trifluoromethyl)imidazo[1,2-a][1,8]naphthyridine-8-carbohydrazide and TsOH (40 mg, 0.210 mmol) (40 wt %) in triethylorthoformate (4 mL, 24.02 mmol) was heated at 80° C. for 1 hour. The mixture was cooled to room temperature, and was purified via reverse phase HPLC to give 8-(1,3,4-oxadiazol-2-yl)-4-(trifluoromethyl)imidazo[1,2-a][1,8]naphthyridin-2-ol (20 mg, 0.059 mmol, 1.35% yield) as a light brown solid. ES LC-MS m/z=322.22 (M+H)⁺.

### Step E: 2-[2-chloro-4-(trifluoromethyl)imidazo[1,2-a]1,8-naphthyridin-8-yl]-1,3,4-oxadiazole

To a solution of 8-(1,3,4-oxadiazol-2-yl)-4-(trifluoromethyl)imidazo[1,2-a][1,8]naphthyridin-2-ol (100 mg, 0.311 mmol) dissolved in N,N-dimethylformamide (3 mL) at room temperature was added POCl₃ (0.058 mL, 0.623 mmol) dropwise. The reaction mixture was stirred at 80° C. for 5 hours, cooled to room temperature, and diluted with water. The brown precipitate was filtered off and purified via reverse phase HPLC to give 2-[2-chloro-4-(trifluoromethyl)imidazo[1,2-a]1,8-naphthyridin-8-yl]-1,3,4-oxadiazole (8.3 mg, 0.023 mmol, 7.46% yield) as a light yellow solid. ¹H NMR (400 MHz, DMSO-d₆) δ: ppm 7.91; (dd, J=9.76, 1.76 Hz, 1 H) 7.98-8.02; (m, 1 H) 8.29; (s, 1 H) 9.16; (s, 1 H) 9.44; (s, 1 H); ES LC-MS m/z=340.16 (M+H)⁺.

### Example 19

### 2-[2,4-bis(propan-2-yl)imidazo[1,2-a]1,8-naphthyridin-8-yl]-1,3,4-oxadiazole

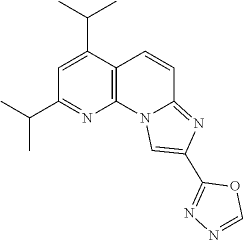 [SMILES: CC(C)c1cc(C(C)C)c2ccc3nc(-c4nnco4)cn3c2n1]

### Step A: 7-amino-2-isopropyl-1,8-naphthyridin-4(1H)-one

Pyridine-2,6-diamine (15.0 g, 137 mmol) and ethyl 4-methyl-3-oxopentanoate (30.6 mL, 190 mmol) were added to diphenyl ether (150 mL). The mixture was heated at 150° C. for 4 hours. The mixture was then heated to 230° C. and excess ethyl 4-methyl-3-oxopentanoate was distilled off using a short path condenser. After ˜30 minutes, the short path condenser was replaced with a reflux condenser and the mixture continued to heat at 230° C. overnight. The mixture was allowed to cool to room temperature. Solids began to precipitate. Ethyl ether was added and then hexanes until a free-flowing solid was observed. The mixture was cooled to 0° C. in an ice-bath and the solids collected by filtration. The solids were washed with cold ether and dried to give the title compound (14.3 g, 47%) as tan solids. ES LC-MS m/z=204 (M+H)⁺.

Step B: 5-bromo-7-isopropyl-1,8-naphthyridin-2-amine

7-amino-2-isopropyl-1,8-naphthyridin-4(1H)-one (6.00 g, 29.5 mmol) was slurried in acetonitrile (60 mL) and phosphorus oxybromide (16.1 g, 56.1 mmol) added. An exotherm was observed. The mixture was heated to 80° C. for 3 hours, then allowed to cool to room temperature and stirred overnight. The mixture was poured into ice and made basic with saturated sodium bicarbonate. The mixture was extracted 3 times with ethyl acetate. The combined organic layers were washed with brine, dried over sodium sulfate, concentrated, and the residue dried under vacuum to give the title compound (5.2 g, 60%) as a rust-colored solid. ES LC-MS m/z=266, 268 (M+H)⁺.

### Step C: ethyl 4-bromo-2-isopropylimidazo[1,2-a][1,8]naphthyridine-8-carboxylate

5-bromo-7-isopropyl-1,8-naphthyridin-2-amine (5.3 g, 20 mmol) and ethyl bromopyruvate (5.01 mL, 39.8 mmol) in ethanol (200 mL) were heated to 80° C. for 2 hours. N,N-diisopropylethylamine (13.9 mL, 80.0 mmol) was added and the reaction continued to heat at 80° C. for 2 hours. The mixture was allowed to cool to room temperature and was concentrated.

The residue was purified by silica chromatography eluting with a gradient of 0% to 30% ethyl acetate in dichloromethane. Fractions were concentrated to give the title compound (2.83 g, 39%) as a pale yellow solid. ES LC-MS m/z=362, 364 (M+H)⁺.

### Step D: lithium 4-bromo-2-isopropylimidazo[1,2-a][1,8]naphthyridine-8-carboxylate

Ethyl 4-bromo-2-isopropylimidazo[1,2-a][1,8]naphthyridine-8-carboxylate (2.8 g, 7.7 mmol) was dissolved in tetrahydrofuran (20 mL) and methanol (20 mL) before a solution of lithium hydroxide monohydrate (0.39 g, 9.3 mmol) in water (20 mL) was added. The mixture was stirred at room temperature overnight and concentrated. The residue was co-evaporated 2 times with toluene and concentrated to give the title compound (2.79 g, >99%) as a tan solid. ES LC-MS m/z=334, 336 (M+H)⁺.

### Step E: 4-bromo-2-isopropylimidazo[1,2-a][1,8]naphthyridine-8-carbohydrazide

Thionyl chloride (50 mL, 685 mmol) was added to lithium 4-bromo-2-isopropylimidazo[1,2-a][1,8]naphthyridine-8-carboxylate (2.7 g, 7.5 mmol) and the mixture heated at 80° C. for 1 hour. The mixture was concentrated and the residue co-evaporated 2 times with toluene. The residue was dissolved in tetrahydrofuran (40 mL) and added to a stirring solution of hydrazine (4.7 mL, 150 mmol) and N,N-diisopropylethylamine (3.91 mL, 22.39 mmol) in tetrahydrofuran (40 mL). After stirring for 1 hour at room temperature, the mixture was concentrated, the residue quenched with water, and the mixture extracted 2 times with dichloromethane. The combined organic layers were washed with brine, dried over sodium sulfate, and concentrated to give the title compound (2.43 g, 82% pure, 77%). ES LC-MS m/z=348, 350 (M+H)⁺.

### Step F: 2-(4-bromo-2-isopropylimidazo[1,2-a][1,8]naphthyridin-8-yI)-1,3,4-oxadiazole

4-bromo-2-isopropylimidazo[1,2-a][1,8]naphthyridine-8-carbohydrazide (2.43 g, 5.72 mmol), p-toluenesulfonic acid monohydrate (1.09 g, 5.72 mmol), and triethyl orthoformate (95 ml, 570 mmol) were heated at 80° C. for 2 hours. The mixture was allowed to cool to room temperature and was concentrated. The residue was purified by silica chromatography eluting with a gradient of 0% to 100% ethyl acetate in dichloromethane. Fractions were concentrated to give the title compound (1.4 g, 65%) as a pale yellow solid. ES LC-MS m/z=358, 360 (M+H)⁺.

### Step G: 2-(2-isopropyl-4-(prop-1-en-2-yl)imidazo[1,2-a][1,8]naphthyridin-8-yl)-1,3,4-oxadiazole

2-(4-bromo-2-isopropylimidazo[1,2-a][1,8]naphthyridin-8-yl)-1,3,4-oxadiazole (75 mg, 0.19 mmol), potassium phosphate (164 mg, 0.771 mmol), potassium trifluoro(prop-1-en-2-yl)borate (57.0 mg, 0.385 mmol), and PdCl₂(dppf)-CH₂Cl₂ adduct (15.7 mg, 0.019 mmol) in 1,4-dioxane (2 mL) and water (0.500 mL) were degassed with nitrogen for 5 minutes before being heated at 90° C. for 3 hours. The mixture was allowed to cool to room temperature and was quenched with water. The mixture was extracted 2 times with ethyl acetate. The combined organic layers were washed with brine, dried over sodium sulfate, concentrated, and the residue purified by silica chromatography eluting with a gradient of 0% to 100% ethyl acetate in dichloromethane. Fractions were concentrated to give the title compound (40 mg, 61%) as an off-white solid. ES LC-MS m/z=320 (M+H)⁺. ¹H NMR (400 MHz, DMSO-d₆) δ ppm 9.40; (s, 1 H), 9.12; (s, 1 H), 7.86; (d, 1 H), 7.69; (d, 1 H), 7.52; (s, 1 H), 5.61; (t, 1 H), 5.15; (s, 1 H), 3.18-3.31; (m, 1 H), 2.22; (s, 3 H), 1.39; (d, 6 H).

### Step H: 2-[2,4-bis(propan-2-yl)imidazo[1,2-a]1,8-naphthyridin-8-yl]-1,3,4-oxadiazole

2-(2-isopropyl-4-(prop-1-en-2-yl)imidazo[1,2-a][1,8]naphthyridin-8-yl)-1,3,4-oxadiazole (32 mg, 0.100 mmol), 10% palladium on carbon (Degussa) (10.66 mg, 10.02 μmol), and acetic acid (0.011 mL, 0.200 mmol) in ethanol (1 mL) and tetrahydrofuran (1 mL) were hydrogenated under balloon pressure for 5 hours. The catalyst was filtered off over celite and the filtrate concentrated. The residue was purified by silica chromatography eluting with a gradient of 0% to 100% ethyl acetate in dichloromethane. Fractions were concentrated to give the title compound (23 mg, 71%) as a white solid. LC-MS m/z=322 (M+H)⁺. ¹H NMR (400 MHz, DMSO-d6) δ ppm 9.39; (s, 1 H), 9.10; (s, 1 H), 8.10; (d, 1 H), 7.70; (d, 1 H), 7.55; (s, 1 H), 3.66-3.90; (m, 1 H), 3.21-3.31; (m, 1 H), 1.31-1.43; (m, 12 H).

### Example 20

### 2-[n4-phenyl-2-(propan-2-yl)imidazo[1,2-a]1,8-naphthyridin-8-yl]-1,3,4-oxadiazole

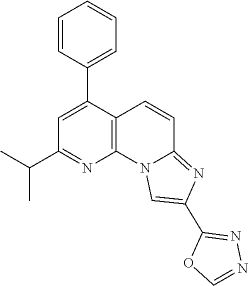 [SMILES: CC(C)c1cc(-c2ccccc2)c2ccc3nc(-c4nnco4)cn3c2n1]

2-(4-bromo-2-isopropylimidazo[1,2-a][1,8]naphthyridin-8-yl)-1,3,4-oxadiazole (51 mg, 0.13 mmol), potassium phosphate tribasic (111 mg, 0.524 mmol), phenylboronic acid (31.9 mg, 0.262 mmol), and PdCl₂ (dppf)-CH2Cl2 adduct (10.7 mg, 0.013 mmol) in 1,4-dioxane (2 mL) and water (0.500 mL) were degassed with nitrogen for 5 minutes before being heated at 90° C. for 3 hours. The mixture was allowed to cool to room temperature and was quenched with water. The mixture was extracted 2 times with ethyl acetate. The combined organic layers were washed with brine, dried over sodium sulfate, concentrated, and the residue purified by silica chromatography eluting with a gradient of 0% to 100% ethyl acetate in dichloromethane. Fractions were concentrated to give the title compound (32 mg, 69%). LC-MS m/z=356 (M+H)⁺. ¹H NMR (400 MHz, DMSO-d6) δ ppm 9.41; (s, 1 H), 9.18; (s, 1 H), 7.67; (d, 2 H), 7.55-7.66; (m, 6 H), 3.35; (s, 1 H), 1.43; (d, 6 H).

The exemplified compounds contain one or more basic groups and are capable of forming a pharmaceutically acceptable acid addition salt by treatment with a suitable acid. Suitable acids include pharmaceutically acceptable inorganic acids and pharmaceutically acceptable organic acids. Such acid addition salts can be formed by reaction of the basic group with the appropriate acid, optionally in a suitable solvent such as an organic solvent, to give the salt which can be isolated by a variety of methods, including crystallisation and filtration.

In one embodiment, a compound is selected from:
2-[2,4-bis(trifluoromethyl)imidazo[1,2-a]1,8-naphthyridin-8-yl]-1,3,4-oxadiazole free base;
2-[2-cyclopentyl-4-(trifluoromethyl)imidazo[1,2-a]1,8-naphthyridin-8-yl]-1,3,4-oxadiazole free base;
2-[2-(propan-2-yl)-4-(trifluoromethyl)imidazo[1,2-a]1,8-naphthyridin-8-yl]-1,3,4-oxadiazole free base;
5-[2,4-bis(trifluoromethyl)imidazo[1,2-a]1,8-naphthyridin-8-yl]-1,3-oxazole free base;
2-[2-cyclopropyl-4-(trifluoromethyl)imidazo[1,2-a]1,8-naphthyridin-8-yl]-1,3,4-oxadiazole free base;
2-[2-methyl-4-(trifluoromethyl)imidazo[1,2-a]1,8-naphthyridin-8-yl]-1,3,4-oxadiazole free base;
2-[9-methyl-2,4-bis(trifluoromethyl)imidazo[1,2-a]1,8-naphthyridin-8-yl]-1,3,4-oxadiazole free base
2-[2-ethoxy-4-(trifluoromethyl)imidazo[1,2-a]1,8-naphthyridin-8-yl]-1,3,4-oxadiazole free base;
2-(2,4-bis(trifluoromethyl)imidazo[1′2′:1,6]pyrido[2,3-d]pyrimidin-8-yl)-1,3,4-oxadiazole free base;
2-[2-ethyl-4-(trifluoromethyl)imidazo[1,2-a]1,8-naphthyridin-8-yl]-1,3,4-oxadiazole free base;
2-[2-phenyl-4-(trifluoromethyl)imidazo[1,2-a]1,8-naphthyridin-8-yl]-1,3,4-oxadiazole free base;
2-[9-chloro-2,4-bis(trifluoromethyl)imidazo[1,2-a]1,8-naphthyridin-8-yl]-1,3,4-oxadiazole free base;
2-[2-(difluoromethoxy)-4-(trifluoromethyl)imidazo[1,2-a]1,8-naphthyridin-8-yl]-1,3,4-oxadiazole free base;
2-[4-chloro-2-(propan-2-yl)imidazo[1,2-a]1,8-naphthyridin-8-yl]-1,3,4-oxadiazole free base;
8-(furan-2-yl)-2,4-bis(trifluoromethyl)imidazo[1,2-a]1,8-naphthyridine free base;
2-{2,4-dimethylimidazo[1,2-a]1,8-naphthyridin-8-yl}-1,3,4-oxadiazole free base;
2-[2,4-bis(trifluoromethyl)imidazo[1,2-a]1,8-naphthyridin-8-yl]-1,3-oxazole free base;
2-[2-chloro-4-(trifluoromethyl)imidazo[1,2-a]1,8-naphthyridin-8-yl]-1,3,4-oxadiazole free base;
2-[2,4-bis(propan-2-yl)imidazo[1,2-a]1,8-naphthyridin-8-yl]-1,3,4-oxadiazole free base; and
2-[4-phenyl-2-(propan-2-yl)imidazo[1,2-a]1,8-naphthyridin-8-yl]-1,3,4-oxadiazole free base, is used in accordance with the invention.

In one embodiment, a compound selected from:
2-[2,4-bis(trifluoromethyl)imidazo[1,2-a]1,8-naphthyridin-8-yl]-1,3,4-oxadiazole free base;
2-(2,4-bis(trifluoromethyl)imidazo[1′,2′:1,6]pyrido[2,3-d]pyrimidin-8-yl)-1,3,4-oxadiazole free base; and
2-[2-cyclopentyl-4-(trifluoromethyl)imidazo[1,2-a]1,8-naphthyridin-8-yl]-1,3,4-oxadiazole free base, and is used in accordance with the invention.

In one embodiment, the uses and methods described herein use 2-[2,4-bis(trifluoromethyl)imidazo[1,2-a]1,8-naphthyridin-8-yl]-1,3,4-oxadiazole, or a pharmaceutically acceptable salt thereof. In a more particular embodiment, the uses and methods use 2-[2,4-bis(trifluoromethyl)imidazo[1,2-a]1,8-naphthyridin-8-yl]-1,3,4-oxadiazole free base.

In one embodiment, the uses and methods described herein use 2-(2,4-bis(trifluoromethyl)imidazo[1′,2′:1,6]pyrido[2,3-d]pyrimidin-8-yl)-1,3,4-oxadiazole, or a pharmaceutically acceptable salt thereof. In a more particular embodiment, the uses and methods use 2-(2,4-bis(trifluoromethyl)imidazo[1′,2′:1,6]pyrido[2,3-d]pyrimidin-8-yl)-1,3,4-oxadiazole free base.

In one embodiment, the uses and methods described herein use 2-[2-cyclopentyl-4-(trifluoromethyl)imidazo[1,2-a]1,8-naphthyridin-8-yl]-1,3,4-oxadiazole, or a pharmaceutically acceptable salt thereof. In a more particular embodiment, the uses and methods use 2-[2-cyclopentyl-4-(trifluoromethyl)imidazo[1,2-a]1,8-naphthyridin-8-yl]-1,3,4-oxadiazole free base.

### Pharmaceutical Compositions/Routes of Administration/Dosages

The compounds described herein, more particularly 2-[2,4-bis(trifluoromethyl)imidazo[1,2-a]1,8-naphthyridin-8-yl]-1,3,4-oxadiazole, or a pharmaceutically acceptable salt thereof; 2-(2,4-bis(trifluoromethyl)imidazo[1′,2′:1,6]pyrido[2,3-d]pyrimidin-8-yl)-1,3,4-oxadiazole, or a pharmaceutically acceptable salt thereof; 2-[2-cyclopentyl-4-(trifluoromethyl)imidazo[1,2-a]1,8-naphthyridin-8-yl]-1,3,4-oxadiazole, or a pharmaceutically acceptable salt thereof, may be administered by any convenient route. In particular embodiments, the compound or pharmaceutically acceptable salt thereof may be administered by inhalation, orally, parenterally or intranasally.

In one embodiment, the compound or pharmaceutically acceptable salt is administered in a pharmaceutical composition containing the compound or pharmaceutically acceptable salt and a pharmaceutically acceptable excipient.

In one embodiment, the compound or pharmaceutically acceptable salt is formulated in a pharmaceutical composition adapted for oral or parenteral administration, or for administration intranasally or by inhalation.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical formulations adapted for nasal administration can comprise a coarse powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the compound or pharmaceutically acceptable salt thereof.

Pharmaceutical formulations adapted for administration by inhalation include fine particle dusts or mists, which may be generated by means of various types of metered, dose pressurized aerosols, nebulizers or insufflators.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations described herein may include other agents conventional in the art having regard to the type of formulation in question, for example those suitable for oral administration may include flavouring agents.

The present invention also provides unitary pharmaceutical compositions in which the compound or pharmaceutically acceptable salt thereof of the present invention and one or more other pharmaceutically active agent(s) may be administered together or separately. In one embodiment, the pharmaceutical composition contains a compound as described here, or a pharmaceutically acceptable salt thereof, and one or more antiviral agents. In one embodiment, the pharmaceutical composition contains a compound, or pharmaceutically acceptable salt thereof, selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole, and one or more antiviral agents or steroids.

In one embodiment, the pharmaceutical composition contains a compound described herein or a pharmaceutically acceptable salt thereof, and one or more other antiviral agents. In one embodiment, the pharmaceutical composition contains a compound, or pharmaceutically acceptable salt thereof, selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole, and one or more other antiviral agents.

In one embodiment, the anti-viral agents are selected from the group consisting of: oseltamivir, remdesivir, ganciclovir, lopinavir, ritonavir and zanamivir. In one embodiment, the pharmaceutical composition contains a single anti-viral agent. In a more particular embodiment, the single anti-viral agent is remdesivir.

In one embodiment, the pharmaceutical composition contains a compound as described herein, or a pharmaceutically acceptable salt thereof, and one or more steroids. In one embodiment, the pharmaceutical composition contains a compound, or a pharmaceutically acceptable salt thereof, selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole, or a pharamaceutically acceptable salt thereof, and one or more steroids.

In one embodiment, one or more steroids is selected from the group consisting of: dexamethasone, prednisone, methylprednisone and hydrocortisone. In one embodiment, the pharmaceutical composition contains a single steroid. In a more particular embodiment, the single steroid is dexamethasone.

Appropriate doses will be readily appreciated by those skilled in the art. When a compound as described herein or a compound, or a pharmaceutically acceptable salt thereof, selected from:
2-[2,4-bis(trifluoromethyl)imidazo[1,2-a]1,8-naphthyridin-8-yl]-1,3,4-oxadiazole;
2-(2,4-bis(trifluoromethyl)imidazo[1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole, or a pharamaceutically acceptable salt thereof, is used in combination with a second therapeutic agent, the dose of each compound may differ from that when the compound is used alone.

### Biological Data

### SARS-CoV, SARS-Co-V2 and MERS-CoV Inhibition Protocol and Impact on IFN Pathway

IFN-incompetent Vero (CCL-81) cells and IFN-competent Calu-3 cells for SARS-CoV and SARS-CoV-2 are purchased from the American Type Culture Collection and are cultured in T225 (Corning Life Sciences) flasks in Minimum Essential Medium (MEM, Corning Life Sciences) supplemented with 10% fetal bovine serum (FBS, Hyclone, GE Life Sciences), 1% L-glutamine, 10 mM HEPES (Sigma Aldrich), 1% non-essential amino acids (ThermoFischer Scientific), and 1% penicillin/streptomycin (Corning). IFN-competent MRC5 cells for MERS-CoV (Garijo, R. et al. Scientific Reports 2016, 6, article No. 24722 “Constrained Evolvability of Interferon Suppression in an RNA Virus”) are cultured in T225 (Corning) flasks in Minimum Essential Medium (MEM, Corning Cellgro) supplemented with 10% fetal bovine serum (FBS, Hyclone). Sub-confluent culture is passaged and split every 3 days, not exceeding 16-19 passages. Cells are detached using 0.25% Trypsin/0.5 mM EDTA solution (Sigma Aldrich) and seeded in assay plates for dose response studies 24 hours before treatment. Whole blood is purchased from the New York Blood Center and human PBMCs are isolated using Ficoll Paque Plus (GE Healthcare). CD14 positive monocytes are further isolated using human CD14 microbeads (Miltenyi Biotec). To differentiate cells into macrophages, one of skilled in the art can look to the methods used in Cellular Signalling, Vol. 16, Issue 3, pp 365-374 (March 2004).

### Cell-Based Infection Assays to Test Antiviral Activity

Experiments with infectious viruses are performed under appropriate biocontainment conditions, such as at the National Institute of Allergy and Infectious Disease (NIAID) or similar facility. Virus stocks are all prepared and characterized at such facility. Cells are seeded at the appropriate density in 384-well imaging plates (Aurora) or 96-well plates (Greiner Bio-One) at 20-24 hours prior to treatment using, for example, an automated Multidrop Combi dispenser (ThermoFisher Scientific).

Compounds described herein are individually tested at 8-10 doses in at least two replicates starting at 1-10 μM with a 2- or 3-fold step dilution in each of the cell lines. Each dose is added directly to the assay wells from a 10 mM stock solution in 100% DMSO using a HP D300 digital dispenser (Hewlett Packard). The final DMSO concentration in each well is normalized to 1%. On each plate, sixteen wells are not infected with virus and serve as a ‘no virus’ control for normalization (0% virus infection). Additional sixteen wells are infected with virus but treated only with 1% DMSO and serve as a high infection control for normalization (100% virus infection). Two hours after treatment assay plates are transferred to the biosafety level (BSL)-3 or 4 suite and are inoculated with the coronavirus being tested at an appropriate multiplicity of infection (MOI). The MOI can be calculated based on the average doubling time of cells (16 hours) and is selected to achieve 60-80% infection rate at the assay endpoint. Following virus inoculation, assay plates are incubated at 37° C. with 5% CO₂ for 20, 24 or 48 hours. To detect expression of viral proteins, the cells infected with SARS-CoV-2 are fixed with 4% paraformaldehyde for 36 hours. Thereafter, the cells are washed three times in phosphate buffer saline (PBS), and then blocked with 1% bovine serum albumin (BSA) at room temperature for 1 hour. Next, the cells are incubated with the primary antibody target NP protein for SARS-CoV-2 ([1:1000 dilution], kindly provided by Prof. Zhengli Shi) for 1 hour. Subsequently, the cells are washed again three times in PBS, and incubated with the secondary antibody (Goat Anti-Rabbit IgG H&L (Cy3®) [1:200 dilution]) (Abcam, USA) for 1 hour. The nuclei are stained with Hoechst 33342 dye (Beyotime, China), and the images are taken by fluorescence microscopy (see Xia, H. et al., Virol. Sinica (2020) 35:355-358), for evaluating antiviral activity.

Compounds with IFN-agonist activity will show increased IFN levels and interferon stimulated gene (ISG) levels in IFN-competent cell lines but not in the IFN-incompetent cell lines (i.e. Vero cells). As a control, pathogen-associated molecular patterns (PAMPs) such as Toll-like receptor (TLR) ligands could be used to stimulate the IFN competent cells (Calu-3) as well as IFN-deficient cells (Vero) and should achieve activation of IFN- signaling in only the IFN-competent cells. ISG levels can be determined by RT-qPCR or DNA microarray analysis. Alternatively, rather than fixation, cell lysates may be prepared for SDS-PAGE and Western blot to examine JAK/STAT phosphorylation as evidence of activation through IFN receptors. To further elucidate the mechanism-of-action (MOA), a JAK inhibitor could be used in conjunction with the compounds of the invention, with intent to abbrogate antiviral effects and ISG induction with methods of detection as described herein.

### Assessment of Interferon Stimulated Genes (ISGs) to Assess Impact on IFN Pathway by Compounds of the Invention

Impact of compounds of the invention, such as 2-[2,4-bis(trifluoromethyl)imidazo[1,2-a]1,8-naphthyridin-8-yl]-1,3,4-oxadiazole, or a pharmaceutically acceptable salt thereof, or 2-[2-cyclopentyl-4-(trifluoromethyl)imidazo[1,2-a]1,8-naphthyridin-8-yl]-1,3,4-oxadiazole, or a pharamaceutically acceptable salt thereof, on the IFN pathway can be done by measuring change of ISGs in animal models, such as mice, Syrian hamster, African green monkeys or Rhesus macaques.

Several discovery-based screens to identify antiviral ISGs are typically designed to determine ISG effects on virus replication using infectious viruses that can undergo a full life cycle (e.g. entry, uncoating, genome replication, particle assembly, egress). Alternatively, replication can be assayed in the absence of virus production with replicons, which are viral genomes that are genetically designed to be competent for replication but unable to produce infectious virus.

ISGs are tested by induction of activity against SARS or MERS subgenomic replicons (Ge, F. et al., Virology, 2007 Mar. 30: 360(1): 150-158 “Derivation of a novel SARS-coronavirus replicon cell line and its application for anti-SARS drug screening”). Several ISGs, including, GBP1, IF16, IF127, IRF1, IRF9, ISG20, MX1, OAS1, PKR, and viperin, are cabaple of significantly reducing replicon activity. Viperin, ISG20, and PKR and their enzymatic activities are required for viral activity in various infectious viruses such as Dengue and West Nile virus. Viperin, ISG20, IFITM2 and IFITM3 have been found to be antiviral against fully infectious Dengue virus (DENY), and IFITM proteins have been proposed to target binding, entry, or nucleocapsid uncoating in such viruses and seem to target early life cycle steps of several viruses. ZAP, ISG20, IFIT1and ISG15 ISGs have also been identified as anti-alphavirus effectors. Any such ISG, as well as Oas1 b, Mx1, Pkr, (Ifit3, Isg15, Mda5, Rig-I, Socs1, Stat1, Cxcl10,Ifit3, Isg15, Socs1 and Usp18 can also be monitored in animals to assess the impact of compounds of the invention on the IFN pathway and confirm the IFN-agonist property of compounds of the invention.

For example, a recent mouse model for SARS-CoV-2 has been developed based on an adeno-assocaited virus (AAV)-mediated expression of hACE2. This AAC-hACE2 mouse model supports viral replication and the mice exhibit pathological findings similar to COVID-19 in human patients (see Osraelow B. et al., “Mouse model of SARS-CoV-2 reveals inflammatory role of type I interferon signalling” in J. Exp. Med. (2020) 217(12)“e20201231). The change and effect that compounds of the invention have on ISGs in this AAV-ACE2 mouse model can be used to assess and quantitate the impact of compounds of the invention on the IFN pathyway in SARS-CoV-2.

### Quantitative RT-PCR Assay to Assess Impact on Viral Load by Compounds of the Invention

Total viral mRNAs are isloated intracellularly or from media using Kit extraction Nucleospin DX virus or Qiagen RNeasy isolation kit by the instructions. RNA quantitation is conducted using SuperScript III Platinum One-Step Qauntitative RT-PCR system (Invitrogen 1732-020) using the following primers.

Fwd Primer
(SEQ ID NO: 1)
5′ GACCCCAAAATCAGCGAAAT 3′
Rev Primer
(SEQ ID NO: 2)
5′ TCTGGTTACTGCCAGTTGAATCTG 3′
Probe
(SEQ ID NO: 3)
5′ FAM-ACCCCGCATTACGTTTGGTGGACC-BHQ-1 3′
Or
Fwd Primer
(SEQ ID NO: 4)
5′ TTACAAACATTGGCCGCAAA 3′
Rev Primer
(SEQ ID NO: 5)
5′ GCGCGACATTCCGAAGAA 3′
Probe
(SEQ ID NO: 6)
5′ FAM-ACAATTTGCCCCCAGCGCTTCAG-BHQ-1 3′

These primers and probes are commercially available from Integrated DNA Technologies (Catalogue No. 10006606) and BioSearch Technologies (Catalogue No. KIT-nCoV-PP1-1000). Detailed instructions for performing real-time reverse-transcriptase polymerase-chain-reaction (RT-PCR) assay using these primers have been published by the CDC (https://www.cdc.gov/coronavirus/2019-nCoV/lab/index.html). The fold change for SARS-CoV2 RNA relative to the control/nontreated samples can be calculated by the 2 ΔΔ,CT method, using GAPDH or actin as a reference gene.

### Animal Model Assessment of Compound Impact on Viral Load

Appropriate animal models to assess the impact of compounds described herein on viral load in the animal include golden Syrian hamster, African green monkey and Rhesus macaque, all known to be SARS-CoV-2 competent. Each animal is infected with SARS-CoV-2, SARS-CoV or MERS. Rhesus macaque: Eight adult rhesus macaques are inoculated with a total dose of 2.6×106 TCID50 of SARS-CoV-2 isolate nCoV-WA1-202018 via a combination of intratracheal, intranasal, ocular and oral routes.

Male and female Syrian hamsters, aged 6-10 weeks old. Phosphate-buffered saline (PBS) is used to dilute virus stocks to the desired concentration, and inocula are back-titrated to verify the dose given. Dulbecco's Modified Eagle Medium (DMEM) containing 105 plaque-forming units in 100 μl of SARS-CoV-2 are intranasally inoculated under intraperitoneal ketamine (200 mg/kg) and xylazine (10 mg/kg) anaesthesia. Mock-infected animals are challenged with 100 μl of PBS. Animals are monitored twice daily for clinical signs of disease.

Their body weight and survival are monitored for 14 days post-inoculation (dpi). Five animals in each group are sacrificed at 2 dpi, 4 dpi, and 7 dpi for virological and histolopathological analyses. Remaining animals are sacrificed at 14 dpi. Blood and major organ tissues at necropsy are separated into two parts, one immediately fixed in 10% PBS-buffered formalin, the other immediately frozen at −80° C. until use.

Six adult African green monkeys (or similar number) are challenged with 5.0×105 PFU of the Italy isolate of SARS-CoV-2 (SARS-CoV-2/INMI1-lsolate/2020/Italy) by combined intratracheal (i.t.) and intranasal (i.n.) routes (dose divided equally). A cohort of e.g., three animals will be euthanized at 5 dpi, while the remaining e.g., three animals are held indefinitely. Blood from all animals is sampled on days 0, 2, 3, 4, and 5, and continuing with days 7, 9, 12, 15, and 21 for animals if held past 5 dpi.

Then animals are treated with a compound as described herein, such as 2-[2,4-bis(trifluoromethyl)imidazo[1,2-a]1,8-naphthyridin-8-yl]-1,3,4-oxadiazole, or a pharmaceutically acceptable salt thereof, or 2-[2-cyclopentyl-4-(trifluoromethyl)imidazo[1,2-a]1,8-naphthyridin-8-yl]-1,3,4-oxadiazole, or a pharamaceutically acceptable salt thereof. A Plaque assay or RT-qPCR as described above is done to quantitate viral load versus time, compared to animal not treated with compound. Impact on viral load by compounds of the invention, such as 2-[2,4-bis(trifluoromethyl)imidazo[1,2-a]1,8-naphthyridin-8-yl]-1,3,4-oxadiazole, or a pharmaceutically acceptable salt thereof, or 2-[2-cyclopentyl-4-(trifluoromethyl)imidazo[1,2-a]1,8-naphthyridin-8-yl]-1,3,4-oxadiazole, or a pharamaceutically acceptable salt thereof, can be determined based on reduction of viral mRNA. The fold change for SARS, MERS and/or SARS-CoV2 RNA relative to the control/nontreated samples can be calculated by the 2 AACT method, using GAPDH or actin as a reference gene.

The expected activity in cells will demonstrate cellular penetration by the compounds of the invention. Applicant will also perform a comparison of the coding nucleotide sequence and amino acid sequence from isolates of several severe acute respiratory syndrome coronavirus 2 (SARS-CoV-2) strains. These will be collected from diverse geographical locations including China, Hong Kong, Australia, Japan (some cruise ship subjects) and USA (15 strains). Various strains can also be used in cellular and animal experiments, with examined outcomes of viral load reduction (efficacy) and ISG induction (MOA). Such data will show that the compounds of the invention impact ISGs downstream of the interferon pathyway and that such impact exists across all strains of virus.

### Example 21

### Human Genetic Evidence

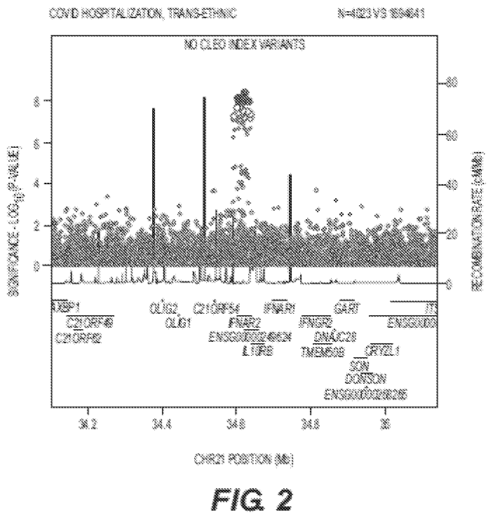

A meta-analysis of genome wide association studies (GWAS) of COVID-19 severity in patients identified an association signal reaching the accepted genome wide statistical threshold of p<5*10⁻⁸ on human chromosome 21 as shown in FIG. 2. FIG. 2 is a regional association plot for COVID-19 hospitalization on chromosome 21 where the x-axis shows physical positions on human genome build GRCh37/hg19 and the y-axes show the −log₁₀ of the p-value for association with COVID-19 risk. Each dot in FIG. 2 represents each of the genetic variants tested for association in the region and human genes in the region are depicted on the lower panel. This observation indicates the locus (genetic region) shown in FIG. 2 plays a role in susceptibility to COVID-19 related hospitalization.

Stated differently, in FIG. 2 the trait analysed was “COVID-19 hospitalization” where “the cases” are individuals being hospitalized with a COVID-19 diagnosis or positive test and “the controls” are individuals who did not report a COVID-19 diagnosis.

A common genetic variant changing the amino acid sequence of the interferon alpha and beta receptor subunit 2 (IFNAR2) was found to be on the credible set of this association. This variant was rs1051393 (dbSNP identifier) located on chromosome 21, at position 34614255 (of the human genome build GRCh37/hg19) and the frequency of the COVID-19 risk increasing allele “G” is 0.32 in the European population. This variant can also be described as NM_207585.2:c.28T>G, NP_997468.1:p.Phe10Val. The variant is also described in FIG. 10 and SEQ ID NO: 12 and SEQ ID NO: 13. Together, this means that IFNAR2 is the gene most likely to be functionally responsible for this genetic association.

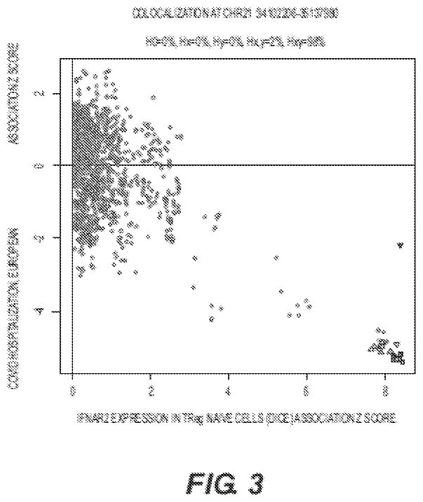

A co-localization analysis between this COVID-19 GWAS association signal and data from expression quantitative trait locus (eQTL) studies was then performed to further investigate the potential genes underlying this COVID-19 association. This analysis revealed the genetic variants associated with increased risk of COVID-19 hospitalization were also associated with the amount of IFNAR2 expression (by using mRNA levels as a proxy for expression of this gene) in multiple tissues. This correlation was observed with eQTLs in multiple cell types relevant to the immune function of IFNAR2, such as T lymphocytes and monocytes. This eQTL data analysis indicated increased COVID-19 hospitalization risk is correlated with decreased IFNAR2 expression as shown in FIG. 3. FIG. 3 shows co-localization between COVID-19 and regulatory T cell eQTL on chromosome 21. In FIG. 3 the x-axis shows the effect of each genetic variant on IFNAR2 expression (measured as the Z-score), while the y-axis shows the effect of each genetic variant on COVID-19 risk [measured as the Z-score). Positive Z-scores indicate the genetic variant increases the value of the trait and vice-versa. Each dot on the plot represents one genetic variant tested in the region. H_{hy} at the top of the figure shows a very high [H_{hy}=98%] posterior probability of co-localization between the COVID-19 and the eQTL associations). This observation indicates that increasing IFNAR2 function with an IFNAR2 agonist will have therapeutic effects on COVID-19 severity.

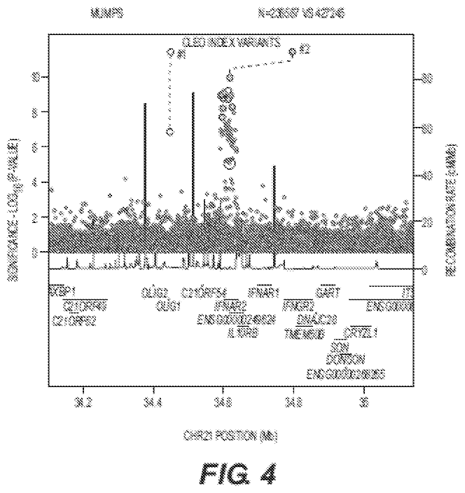

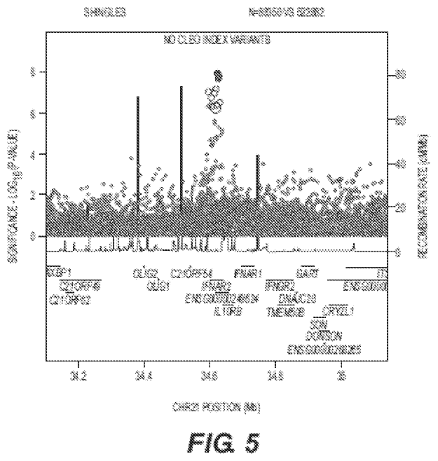

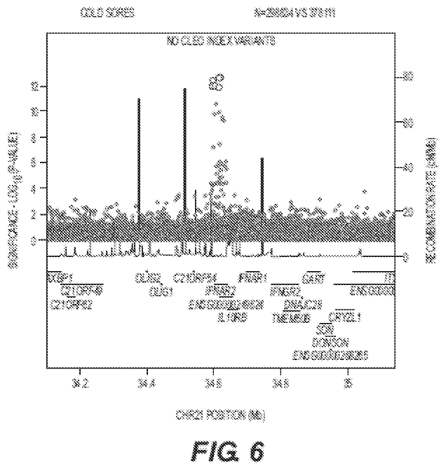

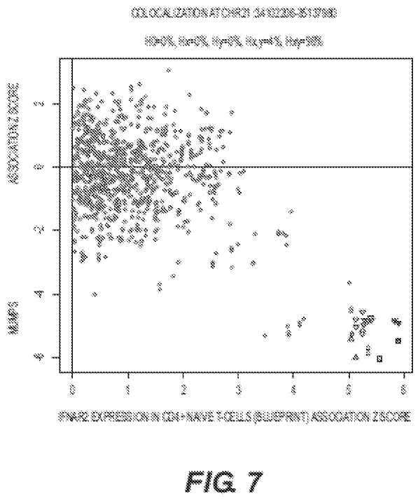

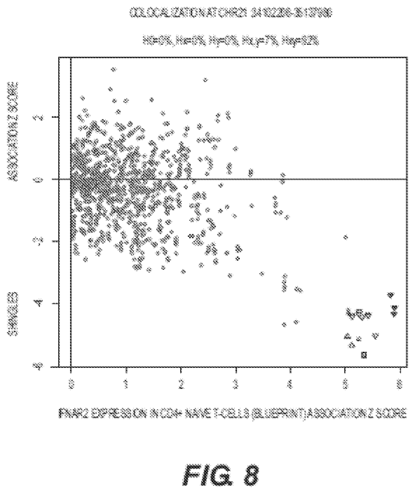

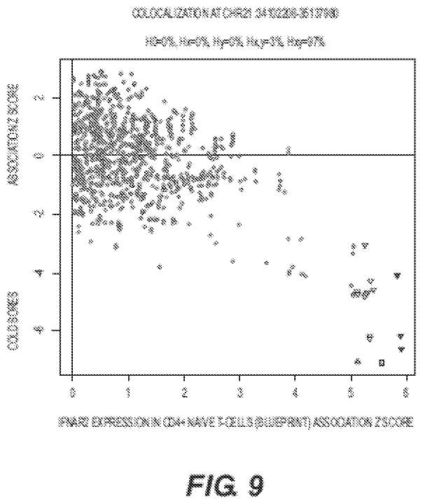

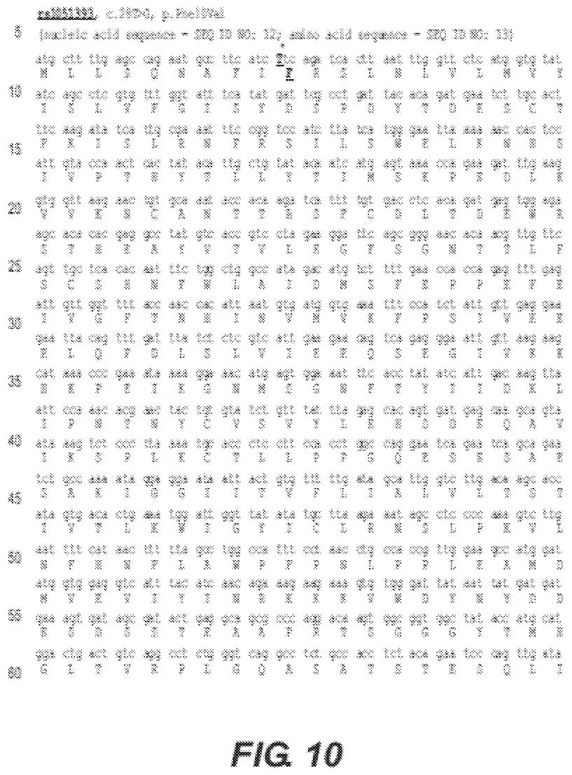

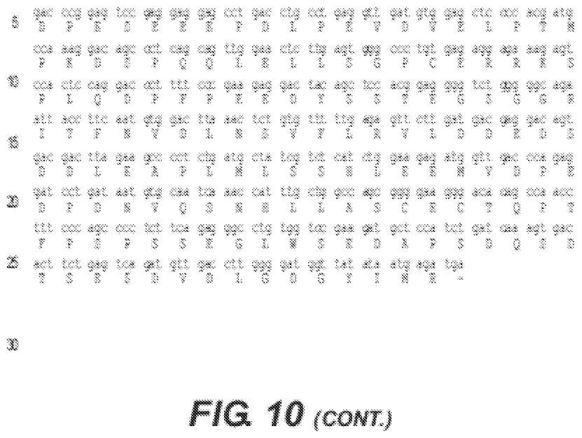

Further association data revealed that the same genetic locus is also associated with susceptibility to other viral diseases like mumps (caused by mumps virus infection), shingles (caused by herpes zoster virus infection) and cold sores (caused by herpes simplex virus type 1 (HSV-1) infection). FIG. 4 FIG. 5 and FIG. 6 are regional association plots of the indicated viral diseases on chromosome 21. These figures have the same structure as described above for FIG. 2 with the difference that the leftmost y-axes show the −log₁₀ of the p value for association with risk of the corresponding viral trait. eQTL co-localization analyses pointed to a correlation between increased risk of these viral diseases and decreased IFNAR2 expression on multiple tissues. FIG. 7 FIG. 8 and FIG. 9 show co-localization between risk of the indicated viral diseases and CD⁴⁺ naïve T cell eQTL on chromosome 21. These figures have the same structure as described above for FIG. 3 with the difference that the y-axes show the Z-score for risk of the corresponding viral disease. These observations indicate that an IFNAR2 agonistic approach will offer therapeutic benefit for mumps, shingles and cold sores.

### Example 22

### Cell-Based Infection Assay to Test Activity Against Human Rhinovirus

Compounds of the present invention will be tested utilizing cytopathic effect (CPE) and cytotoxicity as endpoints. For the CPE assay, human epithelial cells, e.g., HeLa Ohio (European Collection of Authenticated Cell Cultures catalogue number 84121901), will be infected with human rhinovirus, for example HRV-16 strain 11757 (American Type Culture Collection VR-283) at a low multiplicity-of-infection such as 0.01 in the absence or presence of an appropriate concentration range of the compounds that will have been diluted in dimethyl sulfoxide. Cells will be incubated for five days at 33° C. and 5% CO₂. Compounds that elicit an antiviral response will protect the human epithelial cells from CPE induced by infection, and cell viability will be measured using CellTiter-Glo® reagent (CTG) (Promega Corporation, Madison, WI), which is based on luminescent detection of ATP, an indicator of metabolically active cells. Cellular toxicity due to the effect of compound treatment alone will be measured in parallel using the CTG assay with uninfected human epithelial cells. The concentration of compound that inhibits the CPE by 50% (EC₅₀) and causes cytotoxicity (CC₅₀) may be determined using non-linear regression analysis.

### Example 23

### Cell-Based Infection Assay to Test Activity Against Influenza Virus

Compounds of the present invention will be tested utilizing cytopathic effect (CPE) and cytotoxicity as endpoints. For the CPE assay, human epithelial cells, e.g., A549 (American Type Culture Collection CCL-185) will be infected with influenza virus, for example strain A/PR/8/34 (American Type Culture Collection VR-1469) at a low multiplicity of infection such as 0.01 in the absence or presence of an appropriate concentration range of the compounds that will have been diluted in dimethyl sulfoxide. Cells will be incubated for five days at 37° C. and 5% CO₂. Compounds that elicit an antiviral response will protect the human epithelial cells from CPE induced by infection, and cell viability will be measured using CellTiter-Glo® reagent (CTG) (Promega Corporation, Madison, WI), which is based on luminescent detection of ATP, an indicator of metabolically active cells. Cellular toxicity due to the effect of compound treatment alone will be measured in parallel using the CTG assay with uninfected human epithelial cells. The concentration of compound that inhibits the CPE by 50% (EC₅₀) and causes cytotoxicity (CC₅₀) may be determined using non-linear regression analysis.

### Example 24

### Cell-Based Infection Assay to Test Activity against Respiratory Syncytial Virus

Compounds of the present invention will be tested utilizing cytopathic effect (CPE) and cytotoxicity as endpoints. For the CPE assay, human epithelial cells, e.g., HEp-2 cells (European Collection of Authenticated Cell Cultures catalogue number 85011430) will be infected with human respiratory syncytial virus, for example strain A2 (European Collection of

Authenticated Cell Cultures catalogue number 0709161v) at a low multiplicity-of-infection such as 0.01 in the absence or presence of an appropriate concentration range of the compounds that will have been diluted in dimethyl sulfoxide. Cells will be incubated for five days at 37° C. and 5% CO₂. Compounds that elicit an antiviral response will protect the human epithelial cells from CPE induced by infection, and cell viability will be measured using CellTiter-Glo® reagent (CTG) (Promega Corporation, Madison, WI), which is based on luminescent detection of ATP, an indicator of metabolically active cells. Cellular toxicity due to the effect of compound treatment alone will be measured in parallel using the CTG assay with uninfected human epithelial cells. The concentration of compound that inhibits the CPE by 50% (EC₅₀) and causes cytotoxicity (CC₅₀) may be determined using non-linear regression analysis.

### Example 25

Methods: Virus and cells: the Calu-3 cell line used in this study is a clonal isolate, which shows higher growth rate compared with the parental Calu-3 obtained from the American Type Culture Collection (ATCC, HTB-55). Calu-3 was maintained at 37° C. with 5% CO₂ in Eagle's Minimum Essential Medium (EMEM, ATCC) supplemented with 20% heat-inactivated fetal bovine serum (FBS), 1% MEM-Non-Essential Amino Acid solution (Gibco) and 1× Antibiotic-Antimycotic solution (Gibco). SARS-CoV-2 (βCoV/KOR/KCDC03/2020) was provided by Korea Centers for Disease Control and Prevention (KCDC), and was propagated in Vero E6 cells. Viral titers were determined by plaque assays in Vero cells. All experiments using SARS-CoV-2 were performed at Institut Pasteur Korea in compliance with the guidelines of the KNIH, using enhanced biosafety level 3 (BSL-3) containment procedures in laboratories approved for use by the KCDC.

Reagents: Remdesivir (HY-104077) was purchased from MedChemExpress (Monmouth Junction, NJ) and stock solution was dissolved in dimethyl sulfoxide (DMSO) at 10 mM concentration. Anti-SARS-CoV-2 N protein antibody was purchased from Sino Biological Inc (Beijing, China). Alexa Fluor 488 goat anti-rabbit IgG (H+L) secondary antibody and Hoechst 33342 were purchased from Molecular Probes. Paraformaldehyde (PFA) (32% aqueous solution) and normal goat serum were purchased from Electron Microscopy Sciences (Hatfield, PA) and Vector Laboratories, Inc (Burlingame, CA), respectively.

### Dose-Response Curve (DRC) Analysis by Immunofluorescence

Ten-point, three-fold dose-response curves (DRCs) were generated for each drug. Calu-3 cells were seeded at 2.0×10⁴ cells per well with the medium described above in a black, 384-well, μClear plates (Greiner Bio-One), 24 hours before the experiment. Ten-point DRCs were generated, with compound concentrations ranging from 0.0025 to 50 μM. For viral infection, plates were transferred into the BSL-3 containment facility and SARS-CoV-2 was added at a multiplicity of infection of 0.03. The cells were fixed at either 24 hours-post infection (hpi) with 4% paraformaldehyde (PFA), permeabilized with 0.25% tritonX-100 solution. Anti-SARS-CoV-2 Nucleocapsid (N) primary antibody, 488-conjugated goat anti-rabbit IgG secondary antibody and Hoechst 33342 were treated to the cells for immunofluorescence. The images acquired with Operetta high-throughput imaging device (Perkin Elmer) were analyzed using the Columbus software (Perkin Elmer) to quantify cell numbers and infection ratios. Antiviral activity was normalized to infection control (0.5% DMSO) in each assay plate. Cell viability was measured by counting nucleus in each well and normalizing it to the mock control. DRCs were generated using Prism7 software (GraphPad). IC50 values were calculated using nonlinear regression analysis—log[inhibitor] vs. response—Variable slope (four parameters). All IC50 and CC50 values were measured in triplicates. Two DRC graphs were generated from two independent experiments.

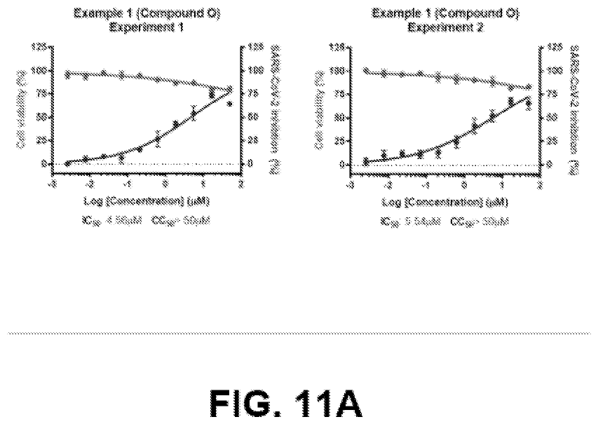

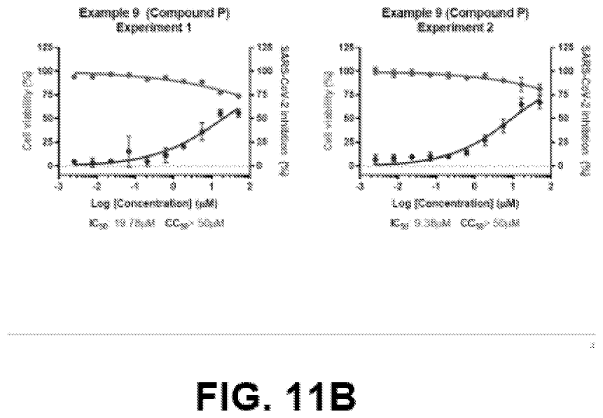

Result DRC graphs are shown in FIG. 11. Example 1 (compound O) and Example 9 (compound P) were tested for anti-SARS-CoV-2 activity in triplicates. Each compound was tested twice independently, resulting in two graphs shown side by side. IC50 and CC50 value of each compound is noted below the X-axis of each graph. Both Example 1 (compound O) and Example 9 (Compound P) showed antiviral activity against SARS-CoV-2 in Calu-3 cells with activity in the μM range. Neither compound showed toxicity in Calu-3 cells for these experiments.

As used herein, Compound O corresponds to Example 1, 2-[2,4-bis(trifluoromethyl)imidazo[1,2-a]1,8-naphthyridin-8-yl]-1 ,3,4-oxadiazole

As used herein, Compound P corresponds to Example 9, 2-(2,4-bis(trifluoromethyl)imidazo[1′2′:1,6]pyrido[2,3-d]pyrimidin-8-yl)-1,3,4-oxadiazole

### Example 26

Example 1 (Compound O) was received as a 10 mM DMSO solution and stored at 4° C. GS-441524 (the predominant metabolite of Remdesivir) was purchased from Carbosynth (United Kingdom) as dry powder, solubilized in DMSO at 10 mM and stored at 4° C. Compound solutions in medium were prepared fresh on the day of treatment at the indicated concentrations with a final DMSO concentration of 0.1%.

SARS-CoV-2 B.1.1.7 (hCoV-19/Belgium/rega-12211513/2020; EPI_ISL_791333, 2020-Dec.-21) was isolated from nasopharyngeal swabs taken from a healthy subject returning to Belgium in December 2020. The virus was passaged twice on VeroE6 cells (stock P2_20210129) and subjected to sequencing on a MinION platform (Oxford Nanopore; Vrancken

B, Wawina-Bokalanga T, Vanmechelen B et al. Accounting for population structure reveals ambiguity in the Zaire Ebolavirus reservoir dynamics. PLoS Negl Trop Dis. 2020 Mar 4; 14(3):e0008117)

SARS-CoV-2 experiments with HAEC-ALI cultures have been described previously (Do TND, Donckers K, Vangeel L et al. A robust SARS-CoV-2 replication model in primary human epithelial cells at the air liquid interface to assess antiviral agents. Antiviral Res. 2021 August; 192:105122). For this experiment MucilAIR bronchial inserts from a healthy donor were received from Epithelix (cat no EPO1 MD batch No. MD0802) in an air-liquid interphase set-up. All cell culture incubations were performed in a cell culture incubator at 37° C. and 5% CO₂. After arrival, the insert was washed with 250 μL pre-warmed MucilAir medium (Epithelix, catalogue no. EPO4MM) and maintained in MucilAir medium for 6 days before use. On the day of the experiment, the HAEC were first pre-treated with basal medium containing compounds at different concentrations for 1 hour, followed by exposing to 100 μL of SARS-CoV-2 inoculum (2×10\hat{ }3 TCID50/insert) at the apical side for 1.5 hour after which the inoculum was removed. The first apical wash with MucilAir medium was performed 24 h later (day 1 postinfection (p. i.)) but this sample was not retained for analysis. Every other day from day 0-7, subsequent apical washes (250 μL) were collected whereas compound-containing medium in the basolateral side of the HAEC culture was refreshed. Wash fluid was stored at −80° C. for analysis. Every other day from day 7-11, apical washes were collected but the medium in the basolateral side of the HAEC culture was replaced with fresh medium without compound or DMSO. Wash fluid was stored at −80° C. for analysis.

For analysis using RT-qPCR the apical wash was first treated using the Cells-to-cDNA™ II cell lysis buffer kit (Thermo Fisher Scientific, catalogue no. AM8723) to prepare the vRNA. Briefly, 5 μL wash fluid was added to 50 μL lysis buffer, incubated at room temperature (RT) for 10 min and then at 75° C. for 15 min. 150 μL nuclease-free water was additionally added to the mixture prior to RTqPCR. In parallel, a ten-fold serial dilution of corresponding virus stock was treated in the same way to be used as standard for RT-qPCR. The amount of viral RNA expressed as TCID50 equivalent per insert (TCID50e/insert) was quantified by RT-qPCR using iTaq universal probes one-step kit (Bio-Rad, catalogue no. 1725141), and a commercial mix of primers for N gene (forward primer 5′GACCCCAAAATCAGCGAAAT-3′, reverse primer 5′-TCTGGTTACTGCCAGTTGAATCTG-3′) and probes (5′-FAM-ACCCCGCATTACGTTTGGTGGACC-BHQ1-3′) manufactured at IDT Technologies (catalogue no. 10006606). The reaction (final volume: 20 μL) consisted of 10 μL one-step reaction mix 2×, 0.5 μL reverse transcriptase, 1.5 ΣL of primers and probes mix, 4 μL nuclease-free water, and 4 μL viral RNA. The RT-qPCR was executed on a Lightcycler 96 thermocycler (Roche), starting at 50° C. for 15 min and 95° C. for 2 min, followed by 45 cycles of 3 seconds at 95° C. and 30 seconds at 55° C. The lower limit of quantification (LLOQ) is determined by the sample of the standard curve that is still in the linear range and has the highest Ct value.

All statistical comparisons in the study were performed in GraphPad Prism (GraphPad Software, Inc.). Statistical significance was determined using the ordinary one-way ANOVA with Sidaks single comparisons test with a singled pooled variance. P-values of ≤0.05 were considered statistically significant. In the figures “ns” indicates a p>0.05. Asterisks indicate a statistical significance level of *p<0.05, **p<0.01, ***p<0.001, ****p<0.0001.

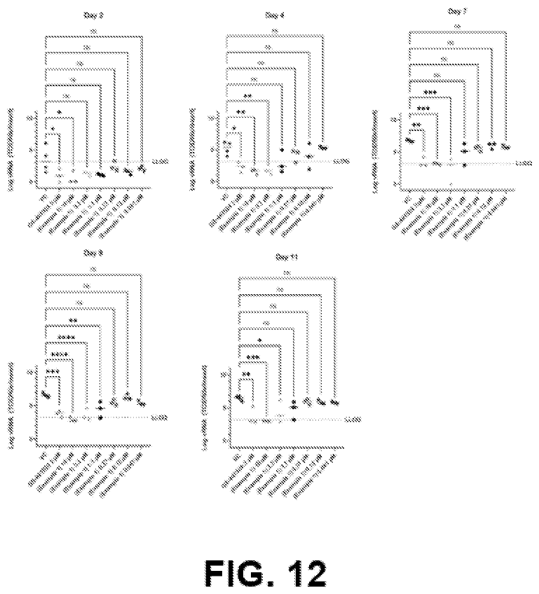

The vRNA of the wash fluids of the different inserts at different timepoints is shown in FIG. 12. There is statistically significant inhibition with GS-441524 at 3 μM and with the compound of Example 1 at 10 μM at all timepoints. There is also statistically significant inhibition with Example 1 at 3 μM (day 4-11) and with Example 1 at 1.1 μM (day 9), indicating that Example 1 is antiviral against SARS-CoV-2 when used in the μM range. No inhibition was observed with Example 1 at 0.37 μM and lower. VC is virus control and contains no compound (only virus).

The compounds of Example 1 and Example 9 were tested in A549-hACE2 SARS-CoV-2 nanoluciferase assay.

A549-hACE2 cells were plated at a density of 20,000 cells/well/100 μl in black-walled clearbottom 96-well plates 24 hr prior to infection. MedChem Express remdesivir was included as a positive control. The compounds were diluted in 100% DMSO (1:3) resulting in a 1000× dose response from 10 to 0.002 mM (10 to 0.002 μM final). All conditions were performed in triplicate. At BSL3, medium was removed, and cells were infected with 100 μl SARS-CoV-2 nLUC (MOI 0.008) for 1 hr at 37° C. after which virus was removed, wells were washed (150 μl) with infection media (DMEM, 4% FBS, 1× antibiotic/antimycotic) and infection media (100 μl ) containing a dose response of drug was added. Plates were incubated at 37° C. for 48 hr. NanoGlo assay was performed 48 hours post infection. Sister plates were exposed to drug but not infected to gauge cytotoxicity via CellTiter-Glo assay, 48hr post treatment.

The compounds of Example 1 and Example 9 were weakly active (1050>1 μM and CC50>10 μM). For analysis of data, efficacy was displayed as % inhibition (referring to percent inhibition of SARS-CoV-2) using the formula 100*(1−(“100% inhibition”−“Experimental”)/(“100% inhibition”−“0% inhibition”)) where “100% inhibition” value is mean of “DMSO No Virus Control” wells from assay Plate 9. As a note for only use of plate 9, this was due to an assay error where virus was mistakenly added to the “DMSO No Virus Control” wells in Plates 1-8. “0% inhibition” value is mean of DMSO+virus wells in each corresponding plate. Inhibition calculations were transferred into Graphpad Prism 8.0.0 for statistical analysis and graphing.

Percent cytotoxicity was calculated using the formula 100*(1−(“100% cytotoxicity”−“Experimental”)/(“100% cytotoxicity”−“0% cytotoxicity”)); where “100% cytotoxicity” value is mean of “DMSO No Cells Control” wells in each corresponding plate and “0% inhibition” value is mean of DMSO+virus wells in each corresponding plate. Cytotoxicity calculations were transferred into Graphpad Prism 8.0.0 for statistical analysis and graphing.

For each compound, inhibition and cytotoxicity data were graphed using nonlinear regression. Nonlinear Regression analysis was run using the equation “log(inhibitor vs. response−Variable slope (four parameters)” with the Equation: Y=Bottom+(Top-Bottom)/(1+10((Log IC50−X)*HillSlope)).

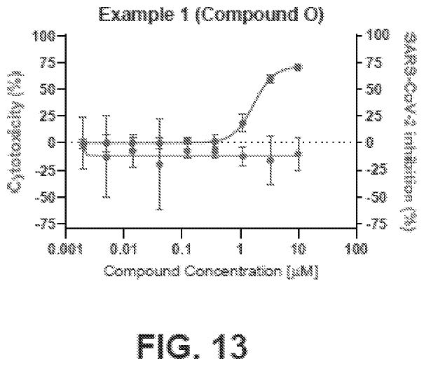

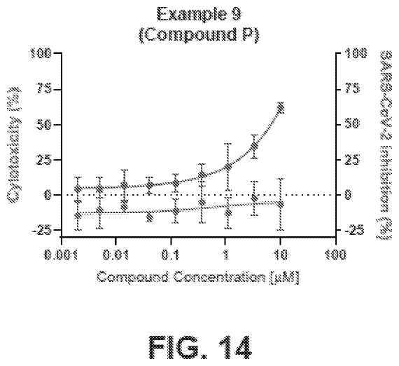

For FIG. 13 and FIG. 14, mean values are depicted and error bars are standard deviation. All data points in triplicate.

FIG. 13 depicts the antiviral activity of the compound of Example 1. Example 1, IC50=1.709 μM. CC50>10 μM. This indicates that the compound of Example 1 has antiviral activity against SARS-CoV-2 reporter virus when used in the μM range in A549-hACE2 cells.

FIG. 14 depicts the antiviral activity of the compound of Example 9. Example 9, IC50 is undefined (top plateau not reached). CC50>10 μM. While a true IC50 value could not be calculated, we can see from FIG. 14 that this compound has antiviral activity against SARS-CoV-2 reporter virus when used in the μM range in A549-hACE2 cells.

SEQUENCES

SEQ ID NO: 1: N1 forward primer
5′ GACCCCAAAATCAGCGAAAT 3′
SEQ ID NO: 2: N1 reverse primer
5′ TCTGGTTACTGCCAGTTGAATCTG 3′
SEQ ID NO: 3: N1 Probe Sequence
5′ FAM-ACCCCGCATTACGTTTGGTGGACC-BHQ-1 3′
SEQ ID NO: 4: N2 forward primer
5′ TTACAAACATTGGCCGCAAA 3′
SEQ ID NO: 5: N2 reverse primer
5′ GCGCGACATTCCGAAGAA 3′
SEQ ID NO: 6 N2 Probe Sequence
5′ FAM-ACAATTTGCCCCCAGCGCTTCAG-BHQ-1 3′
SEQ ID NO: 7: FRET substrate peptide for coronavirus OC43 3CL and 229E protease enzyme
assays
FAM-VARLQSGFG-TAMRA
SEQ ID NO: 8: FRET substrate peptide for SARS coronavirus 3CL protease enzyme assay
FAM-KTSAVLQSGFRKME-TAMRA
SEQ ID NO: 9 FRET substrate peptide for SARS-Coronavirus-2 3CL Protease Enzyme Assay
ESATLQSGLRKAK
SEQ ID NO: 10 Human interferon alpha and beta receptor subunit 2 (IFNAR2) DNA Gene
ID: 3455
ATGCTTTTGAGCCAGAATGCCTTCATCTTCAGATCACTTAATTTGGTTCTCATGGTGTATAT
CAGCCTCGTGTTTGGTATTTCATATGATTCGCCTGATTACACAGATGAATCTTGCACTTTCA
AGATATCATTGCGAAATTTCCGGTCCATCTTATCATGGGAATTAAAAAACCACTCCATTGTA
CCAACTCACTATACATTGCTGTATACAATCATGAGTAAACCAGAAGATTTGAAGGTGGTTAA
GAACTGTGCAAATACCACAAGATCATTTTGTGACCTCACAGATGAGTGGAGAAGCACACAC
GAGGCCTATGTCACCGTCCTAGAAGGATTCAGCGGGAACACAACGTTGTTCAGTTGCTCA
CACAATTTCTGGCTGGCCATAGACATGTCTTTTGAACCACCAGAGTTTGAGATTGTTGGTTT
TACCAACCACATTAATGTGATGGTGAAATTTCCATCTATTGTTGAGGAAGAATTACAGTTTG
ATTTATCTCTCGTCATTGAAGAACAGTCAGAGGGAATTGTTAAGAAGCATAAACCCGAAATA
AAAGGAAACATGAGTGGAAATTTCACCTATATCATTGACAAGTTAATTCCAAACACGAACTA
CTGTGTATCTGTTTATTTAGAGCACAGTGATGAGCAAGCAGTAATAAAGTCTCCCTTAAAAT
GCACCCTCCTTCCACCTGGCCAGGAATCAGAATCAGCAGAATCTGCCAAAATAGGAGGAA
TAATTACTGTGTTTTTGATAGCATTGGTCTTGACAAGCACCATAGTGACACTGAAATGGATT
GGTTATATATGCTTAAGAAATAGCCTCCCCAAAGTCTTGAATTTTCATAACTTTTTAGCCTGG
CCATTTCCTAACCTGCCACCGTTGGAAGCCATGGATATGGTGGAGGTCATTTACATCAACA
GAAAGAAGAAAGTGTGGGATTATAATTATGATGATGAAAGTGATAGCGATACTGAGGCAGC
GCCCAGGACAAGTGGCGGTGGCTATACCATGCATGGACTGACTGTCAGGCCTCTGGGTCA
GGCCTCTGCCACCTCTACAGAATCCCAGTTGATAGACCCGGAGTCCGAGGAGGAGCCTGA
CCTGCCTGAGGTTGATGTGGAGCTCCCCACGATGCCAAAGGACAGCCCTCAGCAGTTGGA
ACTCTTGAGTGGGCCCTGTGAGAGGAGAAAGAGTCCACTCCAGGACCCTTTTCCCGAAGA
GGACTACAGCTCCACGGAGGGGTCTGGGGGCAGAATTACCTTCAATGTGGACTTAAACTC
TGTGTTTTTGAGAGTTCTTGATGACGAGGACAGTGACGACTTAGAAGCCCCTCTGATGCTA
TCGTCTCATCTGGAAGAGATGGTTGACCCAGAGGATCCTGATAATGTGCAATCAAACCATT
TGCTGGCCAGCGGGGAAGGGACACAGCCAACCTTTCCCAGCCCCTCTTCAGAGGGCCTG
TGGTCCGAAGATGCTCCATCTGATCAAAGTGACACTTCTGAGTCAGATGTTGACCTTGGGG
ATGGTTATATAATGAGATGA
SEQ ID NO: 11 Human interferon alpha and beta receptor subunit 2 (IFNAR2) amino acid
sequence
MLLSQNAFIFRSLNLVLMVYISLVFGISYDSPDYTDESCTFKISLRNFRSILSWELKNHSIVPTHYTLLYTI
MSKPEDLKVVKNCANTTRSFCDLTDEWRSTHEAYVTVLEGFSGNTTLFSCSHNFWLAIDMSFEPPEFE
IVGFTNHINVMVKFPSIVEEELQFDLSLVIEEQSEGIVKKHKPEIKGNMSGNFTYIIDKLIPNTNYCVSVYL
EHSDEQAVIKSPLKCTLLPPGQESESAESAKIGGIITVFLIALVLTSTIVTLKWIGYICLRNSLPKVLNFHNF
LAWPFPNLPPLEAMDMVEVIYINRKKKVWDYNYDDESDSDTEAAPRTSGGGYTMHGLTVRPLGQASA
TSTESQLIDPESEEEPDLPEVDVELPTMPKDSPQQLELLSGPCERRKSPLQDPFPEEDYSSTEGSGGRI
TFNVDLNSVFLRVLDDEDSDDLEAPLMLSSHLEEMVDPEDPDNVQSNHLLASGEGTQPTFPSPSSEGL
WSEDAPSDQSDTSESDVDLGDGYIMR
SEQ ID NO: 12 Human interferon alpha and beta receptor subunit 2 (IFNAR2) rs1051393,
c.28T > G, p.Phe10Val nucleic acid sequence
ATGCTTTTGAGCCAGAATGCCTTCATCTTCAGATCACTTAATTTGGTTCTCATGGTGTATAT
CAGCCTCGTGTTTGGTATTTCATATGATTCGCCTGATTACACAGATGAATCTTGCACTTTCA
AGATATCATTGCGAAATTTCCGGTCCATCTTATCATGGGAATTAAAAAACCACTCCATTGTA
CCAACTCACTATACATTGCTGTATACAATCATGAGTAAACCAGAAGATTTGAAGGTGGTTAA
GAACTGTGCAAATACCACAAGATCATTTTGTGACCTCACAGATGAGTGGAGAAGCACACAC
GAGGCCTATGTCACCGTCCTAGAAGGATTCAGCGGGAACACAACGTTGTTCAGTTGCTCA
CACAATTTCTGGCTGGCCATAGACATGTCTTTTGAACCACCAGAGTTTGAGATTGTTGGTTT
TACCAACCACATTAATGTGATGGTGAAATTTCCATCTATTGTTGAGGAAGAATTACAGTTTG
ATTTATCTCTCGTCATTGAAGAACAGTCAGAGGGAATTGTTAAGAAGCATAAACCCGAAATA
AAAGGAAACATGAGTGGAAATTTCACCTATATCATTGACAAGTTAATTCCAAACACGAACTA
CTGTGTATCTGTTTATTTAGAGCACAGTGATGAGCAAGCAGTAATAAAGTCTCCCTTAAAAT
GCACCCTCCTTCCACCTGGCCAGGAATCAGAATCAGCAGAATCTGCCAAAATAGGAGGAA
TAATTACTGTGTTTTTGATAGCATTGGTCTTGACAAGCACCATAGTGACACTGAAATGGATT
GGTTATATATGCTTAAGAAATAGCCTCCCCAAAGTCTTGAATTTTCATAACTTTTTAGCCTGG
CCATTTCCTAACCTGCCACCGTTGGAAGCCATGGATATGGTGGAGGTCATTTACATCAACA
GAAAGAAGAAAGTGTGGGATTATAATTATGATGATGAAAGTGATAGCGATACTGAGGCAGC
GCCCAGGACAAGTGGCGGTGGCTATACCATGCATGGACTGACTGTCAGGCCTCTGGGTCA
GGCCTCTGCCACCTCTACAGAATCCCAGTTGATAGACCCGGAGTCCGAGGAGGAGCCTGA
CCTGCCTGAGGTTGATGTGGAGCTCCCCACGATGCCAAAGGACAGCCCTCAGCAGTTGGA
ACTCTTGAGTGGGCCCTGTGAGAGGAGAAAGAGTCCACTCCAGGACCCTTTTCCCGAAGA
GGACTACAGCTCCACGGAGGGGTCTGGGGGCAGAATTACCTTCAATGTGGACTTAAACTC
TGTGTTTTTGAGAGTTCTTGATGACGAGGACAGTGACGACTTAGAAGCCCCTCTGATGCTA
TCGTCTCATCTGGAAGAGATGGTTGACCCAGAGGATCCTGATAATGTGCAATCAAACCATT
TGCTGGCCAGCGGGGAAGGGACACAGCCAACCTTTCCCAGCCCCTCTTCAGAGGGCCTG
TGGTCCGAAGATGCTCCATCTGATCAAAGTGACACTTCTGAGTCAGATGTTGACCTTGGGG
ATGGTTATATAATGAGATGA
SEQ ID NO: 13 Human Interferon alpha and beta receptor subunit 2 (IFNAR2) rs1051393,
c.28T > G, p.Phe10Val amino acid sequence
MLLSQNAFIFRSLNLVLMVYISLVFGISYDSPDYTDESCTFKISLRNFRSILSWELKNHSIVPTHYT
LLYTIMSKPEDLKVVKNCANTTRSFCDLTDEWRSTHEAYVTVLEGFSGNTTLFSCSHNFWLAID
MSFEPPEFEIVGFTNHINVMVKFPSIVEEELQFDLSLVIEEQSEGIVKKHKPEIKGNMSGNFTYIID
KLIPNTNYCVSVYLEHSDEQAVIKSPLKCTLLPPGQESESAESAKIGGIITVFLIALVLTSTIVTLKW
IGYICLRNSLPKVLNFHNFLAWPFPNLPPLEAMDMVEVIYINRKKKVWDYNYDDESDSDTEAAP
RTSGGGYTMHGLTVRPLGQASATSTESQLIDPESEEEPDLPEVDVELPTMPKDSPQQLELLSG
PCERRKSPLQDPFPEEDYSSTEGSGGRITFNVDLNSVFLRVLDDEDSDDLEAPLMLSSHLEEM
VDPEDPDNVQSNHLLASGEGTQPTFPSPSSEGLWSEDAPSDQSDTSESDVDLGDGYIMR

1-8. (canceled)

9. A compound agonist of IFNAR2 (SEQ ID NO: 10), or a pharmaceutically acceptable salt thereof, which is selected from:
2-[2,4-bis(trifluoromethyl)imidazo[1,2-a]1,8-naphthyridin-8-yl]-1,3,4-oxadiazole;
2-[2-cyclopentyl-4-(trifluoromethyl)imidazo [1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[1,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo [1,2-a]1,8-naphthyridin-8-yl]-1,3,4-oxadiazole;
2-(2,4-bis(trifluoromethyl)imidazo[1′2′:1,6]pyrido[2,3-d]pyrimidin-8-yl)-1,3,4-oxadiazole;
2-[2-ethyl-4-(trifluoromethyl)imidazo [1,2-a]1,8-naphthyridin-8-yl]-1,3,4-oxadiazole;
2-[2-phenyl-4-(trifluoromethyl)imidazo [1,2-a]1,8-naphthyridin-8-yl]-1,3,4-oxadiazole;
2-[9-chloro-2,4-bis(trifluoromethyl)imidazo [1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2,4-bis(trifluoromethyl)imidazo[1,2-a]1,8-naphthyridin-8-yl]-1,3-oxazole;
2-[2-chloro-4-(trifluoromethyl)imidazo [1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole.

10-29. (canceled)

30. A method for treating or preventing COVID-19 in a subject infected with SARS-CoV-2 or at risk of infection with SARS-CoV-2, the method comprising:
administering a therapeutically effective amount of a compound agonist of IFNAR2 (SEQ ID NO: 10, SEQ ID NO: 11) or IFNAR2 variant (SEQ ID NO: 12, SEQ ID NO: 13), or a pharmaceutically acceptable salt thereof, having the structure according to Formula (I): 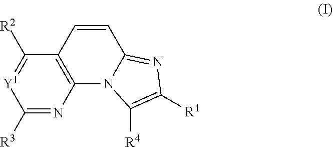
wherein:
Y¹ is selected from the group consisting of CH and N;
R¹ is a 5-membered heteroaryl ring, wherein said 5-membered heteroaryl ring may have one to three heteroatoms selected from N, S, or O, and wherein said 5-membered heteroaryl ring may also be optionally substituted by one to three independent R⁵ groups;
R² is selected from (C₁-C₆)alkyl, (C₁-C₆)alkoxy, halo, and (C₄-C₆)aryl, wherein said R² group may be optionally substituted with one to three R⁵ groups;
R³ is selected from (C₁-C₆)alkyl, (C₁-C₆)alkoxy, halo, (C₄-C₆)aryl, and (C₃-C₆)cycloalkyl, wherein said R³ group may be optionally substituted with one to three R⁵ groups;
R⁴ is selected from hydrogen, (C₁-C₆)alkyl, and halo;
R⁵ is halo or C₁-C₆ alkyl.

31. The method for treating or preventing COVID-19 in a subject infected with SARS-CoV-2 or at risk of infection with SARS-CoV-2 according to claim 30 comprising administering a compound agonist of IFNAR2 or IFNAR2 variant, or pharmaceutically acceptable salt thereof, wherein:
R² is selected from (C₁-C₆)alkyl optionally substituted with one to three R⁵ groups; and
R³ is selected from (C₁-C₆)alkyl optionally substituted with one to three R⁵ groups.

32. The method for treating or preventing COVID-19 in a subject infected with SARS-CoV-2 or at risk of infection with SARS-CoV-2 according to claim 30 comprising administering a compound agonist of IFNAR2 or IFNAR2 variant, or a pharmaceutically acceptable salt thereof, wherein:
R5 is halo.

33. The method for treating or preventing COVID-19 in a subject infected with SARS-CoV-2 or at risk of infection with SARS-CoV-2 according to claim 30 wherein the compound agonist of IFNAR2 or IFNAR2 variant, or a pharmaceutically acceptable salt thereof of claim 1 has the structure of formula (II): 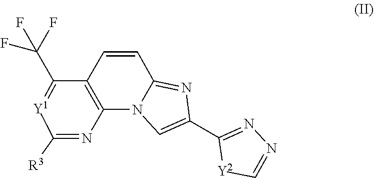
wherein
Y¹ is selected from the group consisting of N and CH;
Y² is selected from the group consisting of O and S; and
R³ is selected from the group consisting of triflouromethyl and cyclopentyl.

34. The method for treating or preventing COVID-19 in a subject infected with SARS-CoV-2 or at risk of infection with SARS-CoV-2 according to claim 33 comprising administering a compound agonist of IFNAR2 or IFNAR2 variant, or a pharmaceutically acceptable salt thereof, of Formula (II), wherein, wherein Y² is O.

35. The method for treating or preventing COVID-19 in a subject infected with SARS-CoV-2 or at risk of infection with SARS-CoV-2 according to claim 33 comprising administering a compound agonist of IFNAR2 or IFNAR2 variant, or a pharmaceutically acceptable salt thereof, which is

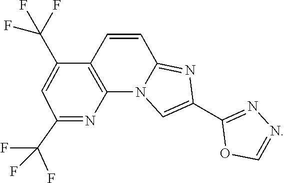 [SMILES: FC(F)(F)c1cc(C(F)(F)F)c2ccc3nc(-c4nnco4)cn3c2n1]

36. The method for treating or preventing COVID-19 in a subject infected with SARS-CoV-2 or at risk of infection with SARS-CoV-2 according to claim 33 comprising administering a compound agonist of IFNAR2 or IFNAR2 variant, or a pharmaceutically acceptable salt thereof, which is

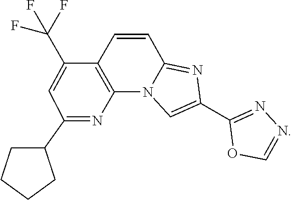 [SMILES: FC(F)(F)c1cc(C2CCCC2)nc2c1ccc1nc(-c3nnco3)cn12]

37. The method for treating or preventing COVID-19 in a subject infected with SARS-CoV-2 or at risk of infection with SARS-CoV-2 according to claim 33 comprising administering a compound agonist of IFNAR2 or IFNAR2 variant, or a pharmaceutically acceptable salt thereof, which is

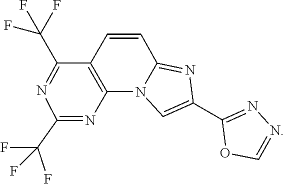 [SMILES: FC(F)(F)c1nc(C(F)(F)F)c2ccc3nc(-c4nnco4)cn3c2n1]

38. A method for treating or preventing COVID-19 in a subject infected with SARS-CoV-2 or at risk of infection with SARS-CoV-2, the method comprising administering a compound, or a pharmaceutically acceptable salt thereof, selected from:
2-[2,4-bis(trifluoromethyl)imidazo[1,2-a]1,8-naphthyridin-8-yl]-1,3,4-oxadiazole;
2-[2-cyclopentyl-4-(trifluoromethyl)imidazo [1,2-a]1,8-naphthyridin-8-yl]-1,3,4-oxadiazole;
2-[2-(propan-2-yl)-4-(trifluoromethyl)imidazo[1,2-a]1,8-naphthyridin-8-yl]-1,3,4-oxadiazole;
5-[2,4-bis(trifluoromethyl)imidazo[1,2-a]1,8-naphthyridin-8-yl]-1,3-oxazole;
2-[2-cyclopropyl-4-(trifluoromethyl)imidazo[1,2-a]1,8-naphthyridin-8-yl]-1,3,4-oxadiazole;
2-[2-methyl-4-(trifluoromethyl)imidazo[1,2-a]1,8-naphthyridin-8-yl]-1,3,4-oxadiazole;
2-[9-methyl-2,4-bis(trifluoromethyl)imidazo[1,2-a]1,8-naphthyridin-8-yl]-1,3,4-oxadiazole;
2-[2-ethoxy-4-(trifluoromethyl)imidazo [1,2-a]1,8-naphthyridin-8-yl]-1,3,4-oxadiazole;
2-(2,4-bis(trifluoromethyl)imidazo [1′2′: 1,6]pyrido[2,3-d]pyrimidin-8-yl)-1,3,4-oxadiazole;
2-[2-ethyl-4-(trifluoromethyl)imidazo [1,2-a]1,8-naphthyridin-8-yl]-1,3,4-oxadiazole;
2-[2-phenyl-4-(trifluoromethyl)imidazo [1,2-a]1,8-naphthyridin-8-yl]-1,3,4-oxadiazole;
2-[9-chloro-2,4-bis(trifluoromethyl)imidazo [1,2-a]1,8-naphthyridin-8-yl]-1,3,4-oxadiazole;
2-[2-(difluoromethoxy)-4-(trifluoromethyl)imidazo[1,2-a]1,8-naphthyridin-8-yl]-1,3,4-oxadiazole;
2-[4-chloro-2-(propan-2-yl)imidazo[1,2-a]1,8-naphthyridin-8-yl]-1,3,4-oxadiazole;
8-(furan-2-yl)-2,4-bis(trifluoromethyl)imidazo[1,2-a]1,8-naphthyridine;
2-{2,4-dimethylimidazo[1,2-a]1,8-naphthyridin-8-yl}-1,3,4-oxadiazole;
2-[2,4-bis(trifluoromethyl)imidazo[1,2-a]1,8-naphthyridin-8-yl]-1,3-oxazole;
2-[2-chloro-4-(trifluoromethyl)imidazo [1,2-a]1,8-naphthyridin-8-yl]-1,3,4-oxadiazole;
2-[2,4-bis(propan-2-yl)imidazo[1,2-a]1,8-naphthyridin-8-yl]-1,3,4-oxadiazole; and
2-[4-phenyl-2-(propan-2-yl)imidazo[1,2-a]1,8-naphthyridin-8-yl]-1,3,4-oxadiazole.

39. A method for treating or preventing COVID-19 in a subject infected with SARS-CoV-2 or at risk of infection with SARS-CoV-2, the method comprising administering a therapeutically effective amount of a compound, or a pharmaceutically acceptable salt thereof, which is selected from:
2-[2,4-bis(trifluoromethyl)imidazo[1,2-a]1,8-naphthyridin-8-yl]-1,3,4-oxadiazole;
2-(2,4-bis(trifluoromethyl)imidazo [1′,2′:1,6]pyrido[2,3-d]pyrimidin-8-yl)-1,3,4-oxadiazole; and
2-[2-cyclopentyl-4-(trifluoromethyl)imidazo[1,2-a]1,8-naphthyridin-8-yl]-1,3,4-oxadiazole.

40. The method according to claim 38, wherein the subject is at risk of infection with SARS-CoV-2 and the method comprises prevention of COVID-19 in the subject at risk of infection with SARS-CoV-2.

41. The method according to claim 38, wherein the subject is a close contact of a patient infected with SARS-CoV-2; in a high risk category; or a healthcare professional.

42. The method according to claim 38, wherein the subject is infected with SARS-CoV-2 and the method comprises treating COVID-19 in the subject infected with SARS-CoV-2.

43. The method according to claim 42, wherein the subject was identified as being infected with SARS-CoV-2 by detection of viral RNA from SARS-CoV-2 from a specimen obtained from the subject.

44. The method according to claim 42, wherein the subject infected with SARS-CoV-2 is infected with a strain (clade) of SARS-CoV-2 selected from the L strain, the S strain, the G strain, the GH strain, the GR strain, the V strain or the O strain of SARS-CoV-2.

45. The method according to claim 44, wherein the subject infected with SARS-CoV-2 is infected with the L strain of SARS-CoV-2.

46. The method according to claim 44, wherein the subject infected with SARS-CoV-2 is infected with the S strain of SARS-CoV-2.

47. The method according to claim 44, wherein the subject infected with SARS-CoV-2 is infected with the G strain of SARS-CoV-2.

48. The method according to claim 44, wherein the subject infected with SARS-CoV-2 is infected with the GH strain of SARS-CoV-2.

49. The method according to claim 44, wherein the subject infected with SARS-CoV-2 is infected with the GR strain of SARS-CoV-2.

50. The method according to claim 44, wherein the subject infected with SARS-CoV-2 is infected with the V strain of SARS-CoV-2.

51. The method according to claim 44, wherein the subject infected with SARS-CoV-2 is infected with the O strain of SARS-CoV-2.

52. The method according to claim 44, wherein COVID-19 in the subject infected with SARS-CoV-2 is associated with pneumonia.

53. The method according to claim 44, wherein COVID-19 in the subject infected with SARS-CoV-2 is associated with acute respiratory distress syndrome.

54. The method according to claim 44, wherein the subject infected with SARS-CoV-2 is undergoing extra-corporeal membrane oxygenation, mechanical ventilation, non-invasive ventilation, or receiving oxygen therapy.

55. The method according to claim 44, wherein the subject infected with SARS-CoV-2 is receiving anti-viral and or steroid treatment.

56. The method according to claim 55, wherein the subject infected with SARS-CoV-2 is receiving an anti-viral agent.

57. The method according to claim 56, wherein the anti-viral agent is selected from remdesivir, ganciclovir, lopinavir, oseltamivir, ritonavir and zanamivir.

58. The method according to claim 44, wherein the compound or pharmaceutically acceptable salt is administered via inhalation.
